(19) United States
(12) Patent Application Publication
(10) Pub. No.: US 2022/0040210 A1
(43) Pub. Date: Feb. 10, 2022

(54) CARBORANE COMPOUNDS, CARBORANE ANALOGS, AND METHODS OF USE THEREOF

(71) Applicant: Ohio State Innovation Foundation, Columbus, OH (US)

(72) Inventors: Christopher Charles COSS, Upper Arlington, OH (US); Chad BENNETT, Columbus, OH (US); Jeffrey PATRICK, Columbus, OH (US); Dasheng WANG, Columbus, OH (US)

(21) Appl. No.: 17/299,618

(22) PCT Filed: Dec. 3, 2019
(86) PCT No.: PCT/US2019/064228

§ 371 Date: Jun. 3, 2021

Related U.S. Application Data
(60) Provisional application No. 62/798,713, filed on Jan. 30, 2019.
(60) Provisional application No. 62/798,711, filed on Jan. 30, 2019.
(60) Provisional application No. 62/798,710, filed on Jan. 30, 2019.
(60) Provisional application No. 62/774,688, filed on Dec. 3, 2018.

(51) Int. Cl. A61K 31/69; A61P 1/16

(52) U.S. Cl. CPC A61K 31/69; A61P 1/16

(57) ABSTRACT

Disclosed are method of treating fibrotic conditions using carboranes and carborane analogs. Also disclosed herein are compounds comprising dicarba-closo-dodecaborane or a dicarba-closo-dodecaborane analog. The compounds can be, for example, estrogen receptor beta (ERβ) agonists. In some examples, the compounds can be selective ERβ agonists. Also provided herein are methods of treating, preventing, or ameliorating cancer in a subject, suppressing tumor growth in a subject, treating an inflammatory disease in a subject, treating a neurodegenerative disease in a subject, treating a psychotropic disorder in a subject, or a combination thereof, by administering to a subject a therapeutically effective amount of one or more of the compounds or compositions described herein, or a pharmaceutically acceptable salt thereof.

### CROSS-REFERENCE TO RELATED APPLICATIONS

This application claims the benefit of U.S. Provisional Application No. 62/774,688, filed Dec. 3, 2018, U.S. Provisional Application No. 62/798,713, filed Jan. 30, 2019, U.S. Provisional Application No. 62/798,710, filed Jan. 30, 2019, and U.S. Provisional Application No. 62/798,711, filed Jan. 30, 2019, each of which is hereby incorporated herein by reference in its entirety.

### BACKGROUND

Estrogen can influence the growth, differentiation, and functioning of many tissues. For example, estrogens play an important role in the female and male reproductive systems, and also in bone maintenance, the central nervous system, and the cardiovascular system. Because of their beneficial actions in non-reproductive tissues, such as bone, brain, and urogenital tract, estrogens would be ideal drugs if they did not have serious adverse effects, such as increasing the risk of breast cancer, endometrial cancer, thromboembolisms, and strokes.

The physiological functions of estrogenic compounds are modulated largely by the estrogen receptor subtypes alpha (ERα) and beta (ERβ). The activity of the two ER subtypes is controlled by the binding of the endogenous hormone 17β-estradiol or of synthetic nonhormonal compounds to the ligand-binding domain.

In humans, both receptor subtypes are expressed in many cells and tissues, and they can control physiological functions in various organ systems, such as reproductive, skeletal, cardiovascular, and central nervous systems, as well as in specific tissues (such as breast and subcompartments of prostate and ovary). ERα is present mainly in mammary glands, uterus, ovary (thecal cells, bone, male reproductive organs (testes and epididumis), prostate (stroma), liver, and adipose tissue. By contrast, ERβ is found mainly in the prostate (epithelium), bladder, ovary (granulosa cells), colon, adipose tissue, and immune system. Both subtypes are markedly expressed in the cardiovascular and central nervous systems, There are some common physiological roles for both estrogen receptor subtypes, such as in the development and function of the ovaries, and in the protection of the cardiovascular system. The alpha subtypes has a more prominent roles on the mammary gland and uterus, as well as on the preservation of skeletal homeostasis and the regulation of metabolism, The beta subtype seems to have a more pronounced effect on the central nervous and immune systems, and it general counteracts the ERα-promoted cell hyperproliferation in tissues such as breast and uterus.

Compounds that either induce or inhibit cellular estrogen responses have potential value as biochemical tools and candidates for drug development. Most estrogen receptor modulators are non-selective for the ER subtypes, but is has been proposed that compounds with ER subtype selectivity would be useful. However, the development of compounds possessing ER subtype specificity still constitutes a major challenge, as the ligand binding domains of the two subtypes are very similar in structure and amino acid sequence.

### SUMMARY

Disclosed herein are methods of treating fibrotic conditions using carboranes and carborane analogs. The carborane and carborane analogs can function as ERβ agonists. In certain embodiments, the fibrotic condition can comprise a fibrotic condition of the liver, such as non-alcoholic fatty liver disease (NAFLD) and non-alcoholic steatohepatitis (NASH).

Also disclosed herein are compounds comprising dicarba-closo-dodecaborane. For example, provided are compounds defined by the formula below, or a pharmaceutically acceptable salt thereof:

A-Q-R¹

wherein Q is a substituted or unsubstituted dicarba-closo-dodecaborane cluster, and A and R¹ are attached to Q in a para configuration; A is a substituted or unsubstituted heteroaryl ring; R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₄-C₂₀ alkylcycloalkyl, substituted or unsubstituted C₁-C₂₀ acyl, C₁-C₂₀ acyl, —C(O)N R³R⁴, —S(O)—R³, —S(O₂)—R³, substituted or unsubstituted C₂-C₂₀ heteroalkyl, or NR³R⁴; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some embodiments, Q is

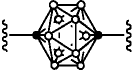

wherein ● is a carbon atom or a boron atom; and ∘ is C—H, C-halogen, C-alkyl, C—OH, C—NH₂, B—H, B-halogen, B-alkyl, B—OH, or B—NH₂.

In some cases, the compound can be defined by the formula below, or a pharmaceutically acceptable salt thereof:

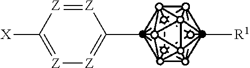

wherein ● is a carbon atom; ∘ is B—H, B-halogen, B-alkyl, B—OH, or B—NH₂; X is OH, NHR², SH, or S(O)(O)NHR²; Z is, individually for each occurrence, N or CH, with the proviso that at least one of Z is N; R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₄-C₂₀ alkylcycloalkyl, substituted or unsubstituted C₁-C₂₀ acyl, C₁-C₂₀ acyl, —C(O)N R³R⁴, —S(O)—R³, —S(O₂)—R³, substituted or unsubstituted C₂-C₂₀ heteroalkyl, or NR³R⁴; R² is H, OH, halogen, or substituted or unsubstituted C₁-C₄ alkyl; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some cases, one of Z can be N. In some cases, two or more of Z can be N. In some cases, three of Z can be N.

In some embodiments, the compound can be defined by one of the formulae below, or a pharmaceutically acceptable salt thereof:

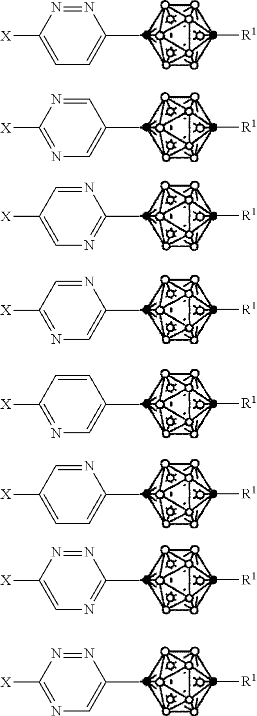

wherein ● is a carbon atom; ∘ is B—H, B-halogen, B-alkyl, B—OH, or B—NH₂; X is OH, NHR², SH, or S(O)(O)NHR²; R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₄-C₂₀ alkylcycloalkyl, substituted or unsubstituted C₁-C₂₀ acyl, C₁-C₂₀ acyl, —C(O)N R³R⁴, —S(O)—R³, —S(O₂)—R³, substituted or unsubstituted C₂-C₂₀ heteroalkyl, or NR³R⁴; R² is H, OH, halogen, or substituted or unsubstituted C₁-C₄ alkyl; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some embodiments, the compound can be defined by one of the formulae below, or a pharmaceutically acceptable salt thereof:

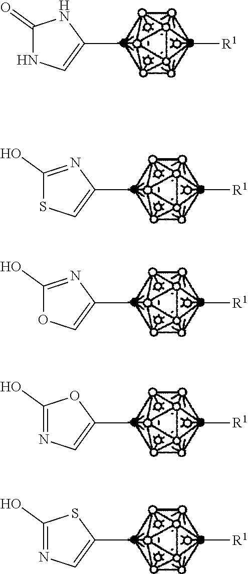

wherein ● is a carbon atom; ∘ is B—H, B-halogen, B-alkyl, B—OH, or B—NH₂; R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₄-C₂₀ alkylcycloalkyl, substituted or unsubstituted C₁-C₂₀ acyl, C₁-C₂₀ acyl, —C(O)N R³R⁴, —S(O)—R³, —S(O₂)—R³, substituted or unsubstituted C₂-C₂₀ heteroalkyl, or NR³R⁴; R² is H, OH, halogen, or substituted or unsubstituted C₁-C₄ alkyl; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some of the embodiments above, X can be OH.

In some of the embodiments above, R¹ can be a substituted or unsubstituted C₆-C₁₀ alkyl (e.g., a C₆-C₁₀ hydroxyalkyl).

In some of the embodiments above, R¹ can be a substituted or unsubstituted C₃-C₁₆ alkylaryl (e.g., a C₃-C₁₆ hydroxyalkylaryl).

In some of the embodiments above, R¹ can be a substituted or unsubstituted C₅-C₂₀ alkylaryl (e.g., a C₅-C₂₀ hydroxyalkylaryl).

In some of the embodiments above, R¹ can be a substituted or unsubstituted C₅-C₁₀ acyl.

In some of the embodiments above, R¹ can be a substituted or unsubstituted branched C₄-C₁₀ alkyl (e.g., a branched C₄-C₁₀ hydroxyalkyl).

In some embodiments, the compound is defined by a formula below, or a pharmaceutically acceptable salt thereof:

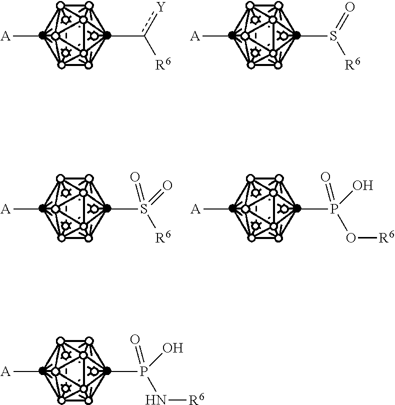

wherein ● is a carbon atom; ∘ is B—H, B-halogen, B-alkyl, B—OH, or B—NH₂; the dotted line to Y indicates that the bond can be a single bond or a double bond, as valence permits; A is a substituted or unsubstituted heteroaryl ring; Y, when present, is O, halogen, OR^{2′}, NHR², SH, or S(O)(O)NHR²; R⁶ is substituted or unsubstituted C₁-C₁₉ alkyl, substituted or unsubstituted C₂-C₁₉ alkenyl, substituted or unsubstituted C₂-C₁₉ alkynyl, substituted or unsubstituted C₂-C₁₉ alkylaryl, substituted or unsubstituted C₄-C₁₉ alkylcycloalkyl, and substituted or unsubstituted C₂-C₂a heteroalkyl. or NR³R⁴; R² is H, OH, halogen, or substituted or unsubstituted C₁-C₄ alkyl; R^{2′} is H or substituted or unsubstituted C₁-C₄ alkyl; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some of these embodiments, Y is OH. In some of these embodiments, Y is F. In some of these embodiments, Y is O.

In some examples, R⁶ can be a substituted or unsubstituted C₃-C₁₀ alkyl, such as a substituted or unsubstituted C₆-C₉ alkyl.

In some examples, R⁶ can be a substituted or unsubstituted C₂-C₁₅ alkylaryl.

In some examples, R⁶ can be a substituted or unsubstituted branched C₂-C₉ alkyl.

In some examples, R⁶ can be a substituted or unsubstituted C₃-C₁₀ heteroalkyl, such as a substituted or unsubstituted C₆-C₉ heteroalkyl.

Also provided are compounds defined by the formula below, or a pharmaceutically acceptable salt thereof:

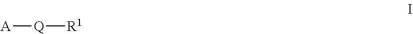

wherein Q is a substituted or unsubstituted dicarba-closo-dodecaborane cluster, and A and R¹ are attached to Q in a para configuration; A is a substituted or unsubstituted aryl ring or a substituted or unsubstituted heteroaryl ring; R¹ is substituted or unsubstituted C₂-C₂₀ heteroalkyl, —C(O)N R³R⁴, —S(O)—R³, —S(O₂)—R³, or NR³R⁴; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl, with the proviso that when present, at least one of R³ and R⁴ is C₂-C₂₀ heteroalkyl.

In some embodiments, Q is

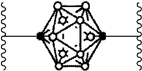

wherein ● is a carbon atom or a boron atom; and ∘ is C—H, C-halogen, C-alkyl, C—OH, C—NH₂, B—H, B-halogen, B-alkyl, B—OH, or B—NH₂.

In some embodiments, the compound can be defined by the formula below, or a pharmaceutically acceptable salt thereof:

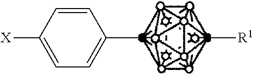

wherein ● is a carbon atom; ∘ is B—H, B-halogen, B-alkyl, B—OH, or B—NH₂; X is OH, NHR², SH, or S(O)(O)NHR²; Z is, individually for each occurrence, N or CH, with the proviso that at least one of Z is N; R¹ is substituted or unsubstituted C₂-C₂₀ heteroalkyl, —C(O)N R³R⁴, —S(O)—R³, —S(O₂)—R³, or NR³R⁴; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl, with the proviso that when present, at least one of R³ and R⁴ is C₂-C₂₀ heteroalkyl.

In some of these embodiments, X can be OH.

Also provided are compounds defined by any of the formula below, or a pharmaceutically acceptable salt thereof:

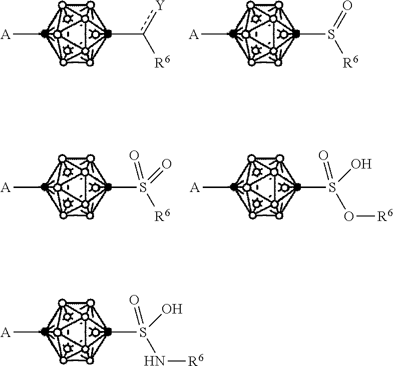

wherein ● is a carbon atom; ∘ is B—H, B-halogen, B-alkyl, B—OH, or B—NH₂; the dotted line to Y indicates that the bond can be a single bond or a double bond, as valence permits; A is a substituted or unsubstituted aryl ring a substituted or unsubstituted heteroaryl ring; Y, when present, is O, halogen, OR^{2′}, NHR², SH, or S(O)(O)NHR²; R⁶ is substituted or unsubstituted C₁-C₁₉ alkyl, substituted or unsubstituted C₂-C₁₉ alkenyl, substituted or unsubstituted C₂-C₁₉ alkynyl, substituted or unsubstituted C₂-C₁₉ alkylaryl, substituted or unsubstituted C₄-C₁₉ alkylcycloalkyl, and substituted or unsubstituted C₂-C₂₀ heteroalkyl. or NR³R⁴; R² is H, OH, halogen, or substituted or unsubstituted C₁-C₄ alkyl; R^{2′} is H or substituted or unsubstituted C₁-C₄ alkyl; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some of these embodiments, Y is OH. In some of these embodiments, Y is F. In some of these embodiments, Y is O.

In some examples, R⁶ can be a substituted or unsubstituted C₃-C₁₀ alkyl, such as a substituted or unsubstituted C₆-C₉ alkyl.

In some examples, R⁶ can be a substituted or unsubstituted C₂-C₁₅ alkylaryl.

In some examples, R⁶ can be a substituted or unsubstituted branched C₂-C₉ alkyl.

In some examples, R⁶ can be a substituted or unsubstituted C₃-C₁₀ heteroalkyl, such as a substituted or unsubstituted C₆-C₉ heteroalkyl.

In some examples, the carborane cluster can include a heteroatom. In some examples, the carborane cluster can include an isotopically labeled atom (i.e., a radiolabeled atom). In some examples, the carborane cluster can include an isotopically labeled Boron atom (e.g., ¹⁰B).

Also disclosed herein are dicarba-closo-dodecaborane analogs. For example, provided herein are compounds defined by the formula below, or a pharmaceutically acceptable salt thereof:

A-Q-R¹

wherein A is a substituted or unsubstituted aryl ring or a substituted or unsubstituted heteroaryl ring; Q is a spacer group chosen from one of the following:

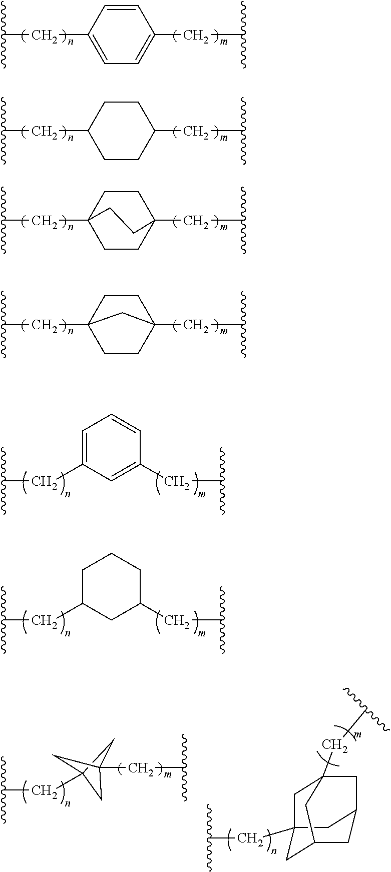

where m and n are each individually 0, 1, 2, or 3; R¹ is substituted or unsubstituted C₄-C₂₀ alkyl, substituted or unsubstituted C₄-C₂₀ heteroalkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₄-C₂₀ alkylcycloalkyl, substituted or unsubstituted C₁-C₂₀ acyl, C₁-C₂₀ acyl, —C(O)N R³R⁴, or NR³R⁴; and R³ and R⁴ are independently selected from substituted or unsubstituted C₁-C₂₀ alkyl, substituted or unsubstituted C₁-C₂₀ heteroalkyl, substituted or unsubstituted C₂-C₂₀ alkenyl, substituted or unsubstituted C₂-C₂₀ alkynyl, substituted or unsubstituted C₂-C₂₀ alkylaryl, or substituted or unsubstituted C₄-C₂₀ alkylcycloalkyl.

In certain embodiments, Q can be chosen from one of the following:

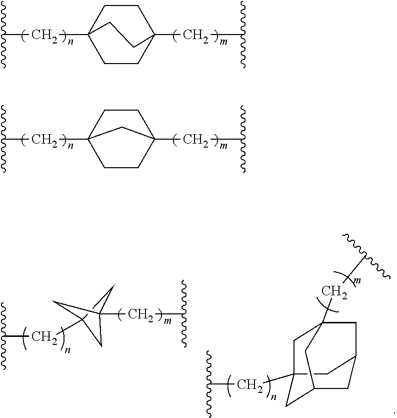

In some embodiments, A is

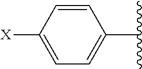

wherein X is OH, NHR², SH, or S(O)(O)NHR² and R² is H, OH, halogen, or substituted or unsubstituted C₁-C₄ alkyl. In some of these embodiments, X is OH.

In some embodiments, A is

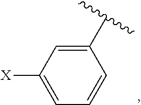

herein X is OH, NHR², SH, or S(O)(O)NHR² and R² is H, OH, halogen, or substituted or unsubstituted C₁-C₄ alkyl. In some of these embodiments, X is OH.

In some embodiments, A is

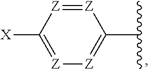

wherein Z is, individually for each occurrence, N or CH, with the proviso that at least one of Z is N; X is OH, NHR², SH, or S(O)(O)NHR²; and R² is H, OH, halogen, or substituted or unsubstituted C₁-C₄ alkyl. In some of these embodiments, A can be one of the following:

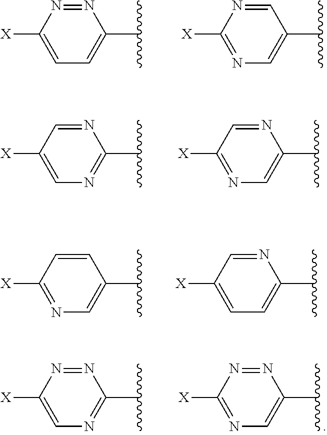

In some of these embodiments, X is OH.

In some embodiments, A is

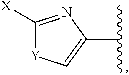

wherein Y is S or O; X is OH, NHR², SH, or S(O)(O)NHR²; and R² is H, OH, halogen, or substituted or unsubstituted C₁-C₄ alkyl. In some of these embodiments, X is OH.

In some embodiments, A is

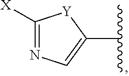

wherein Y is S or O; X is OH, NHR², SH, or S(O)(O)NHR²; and R² is H, OH, halogen, or substituted or unsubstituted C₁-C₄ alkyl. In some of these embodiments, X is OH.

In some embodiments, A is

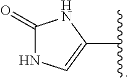 [SMILES: *c1c[nH]c(=O)[nH]1]

In some of the embodiments above, R¹ can be a substituted or unsubstituted C₆-C₁₀ alkyl (e.g., a C₆-C₁₀ hydroxyalkyl).

In some of the embodiments above, R¹ can be a substituted or unsubstituted C₃-C₁₆ alkylaryl (e.g., a C₃-C₁₆ hydroxyalkylaryl).

In some of the embodiments above, R¹ can be a substituted or unsubstituted C₅-C₂₀ alkylaryl (e.g., a C₅-C₂₀ hydroxyalkylaryl).

In some of the embodiments above, R¹ can be a substituted or unsubstituted C₅-C₁₀ acyl.

In some of the embodiments above, R¹ can be a substituted or unsubstituted branched C₄-C₁₀ alkyl (e.g., a branched C₄-C₁₀ hydroxyalkyl).

In some embodiments, R¹ can comprise one of the following

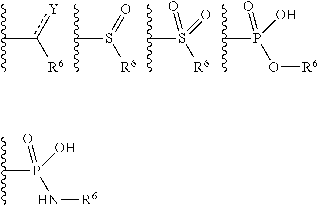

wherein the dotted line to Y indicates that the bond can be a single bond or a double bond, as valence permits; Y, when present, is O, halogen, OR^{2′}, NHR², SH, or S(O)(O)NHR²; R⁶ is substituted or unsubstituted C₁-C₁₉ alkyl, substituted or unsubstituted C₂-C₁₉ alkenyl, substituted or unsubstituted C₂-C₁₉ alkynyl, substituted or unsubstituted C₂-C₁₉ alkylaryl, substituted or unsubstituted C₄-C₁₉ alkylcycloalkyl, and substituted or unsubstituted C₂-C₂₀ heteroalkyl. or NR³R⁴; R² is H, OH, halogen, or substituted or unsubstituted C₁-C₄ alkyl; R^{2′} is H or substituted or unsubstituted C₁-C₄ alkyl; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some of these embodiments, Y is OH. In some of these embodiments, Y is F. In some of these embodiments, Y is O.

In some examples, R⁶ can be a substituted or unsubstituted C₃-C₁₀ alkyl, such as a substituted or unsubstituted C₆-C₉ alkyl.

In some examples, R⁶ can be a substituted or unsubstituted C₂-C₁₅ alkylaryl.

In some examples, R⁶ can be a substituted or unsubstituted branched C₂-C₉ alkyl.

In some examples, R⁶ can be a substituted or unsubstituted C₃-C₁₀ heteroalkyl, such as a substituted or unsubstituted C₆-C₉ heteroalkyl.

In some examples, the compounds disclosed herein can have an EC₅₀ of 800 nM or less at estrogen receptor beta (ERβ). In some examples, the compounds disclosed herein can have an EC₅₀ of 6 nM or less at estrogen receptor beta (ERβ). In some examples, the compounds disclosed herein can have an EC₅₀ in the subnanomolar range (e.g., an EC₅₀ of less than 1 nM, an EC₅₀ of 0.5 nM or less, or an EC₅₀ of 0.1 nM or less).

In some examples, the compounds disclosed herein can have an ERβ-to-ERα agonist ratio of 8 or more. In some examples, the compounds disclosed herein can have an ERβ-to-ERα agonist ratio of 400 or more.

Some compounds disclosed herein have selectivity for ERβ over ERα and thus exert agonist activity on ERβ without undesired effects on ERα. Therefore, the compounds can be used in the treatment of various ERβ-related (ERβ-mediated) diseases, for examples cancers, inflammatory diseases, neurodegenerative diseases, cardiovascular diseases, benign prostate hyperplasia and osteoporosis.

Also provided herein are methods of treating, preventing, or ameliorating cancer in a subject. The methods include administering to a subject a therapeutically effective amount of one or more of the compounds or compositions described herein, or a pharmaceutically acceptable salt thereof. In some examples, the cancer can be selected from the group consisting of breast cancer, colorectal cancer, endometrial cancer, ovarian cancer, and prostate cancer. The methods of treatment or prevention of cancer described herein can further include treatment with one or more additional agents (e.g., an anti-cancer agent or ionizing radiation).

Also described herein are methods of suppressing tumor growth in a subject. The method includes contacting at least a portion of the tumor with a therapeutically effective amount of a compound or composition as described herein, and optionally includes the step of irradiating at least a portion of the tumor with a therapeutically effective amount of ionizing radiation.

Also described herein are methods of treating an inflammatory disease in a subject. The methods can include administering to the subject a therapeutically effective amount of a compound or a composition as described herein. In some examples, the inflammatory disease is selected from the group consisting of arthritis and inflammatory bowel disease. The methods of treatment of inflammatory diseases described herein can further include treatment with one or more additional agents (e.g., an anti-inflammatory agent).

Also disclosed herein are methods of treating a neurodegenerative disease in a subject. The methods can comprise administering to the subject a therapeutically effective amount of a compound or a composition as described herein.

Also disclosed herein are methods of treating a psychotropic disorder in a subject. The methods can comprise administering to the subject a therapeutically effective amount of a compound or a composition as described herein.

Also disclosed herein are methods of imaging a cell or a population of cells expressing ERβ within or about a subject. The methods can comprise administering to the subject an amount of a compound or a composition as described herein; and detecting the compound or the composition.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 illustrates the average body weight change observed in the four study groups over the course of the treatment period.

FIG. 2A is a plot showing the body weight of animals on the day of sacrifice.

FIG. 2B is a plot showing the liver weight of animals on the day of sacrifice.

FIG. 2C is a plot showing the liver-to-body weight ratio of animals on the day of sacrifice.

FIG. 3A is a plot showing plasma alanine aminotransferase (ALT) levels (in U/L) on the day of sacrifice.

FIG. 3B is a plot showing liver triglyceride levels (in mg/g liver) on the day of sacrifice.

FIG. 4 is a plot showing the non-alcoholic fatty liver disease (NAFLD) activity score on the day of sacrifice.

FIG. 5A is a plot showing the steatosis score on the day of sacrifice.

FIG. 5B is a plot showing the inflammation score on the day of sacrifice.

FIG. 5C is a plot showing the ballooning score on the day of sacrifice.

FIG. 6 is a plot showing the fibrosis area (sirius red-positive area, %) on the day of sacrifice.

### DETAILED DESCRIPTION

The compounds, compositions, and methods described herein may be understood more readily by reference to the following detailed description of specific aspects of the disclosed subject matter and the Examples included therein.

Before the present compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

As used herein, “fibrotic condition” refers to a disease or condition involving the formation and/or deposition of fibrous tissue, e.g., excessive connective tissue builds up in a tissue and/or spreads over or replaces normal organ tissue (reviewed in, e.g., Wynn, Nature Reviews 4:583-594 (2004) and Abdel-Wahab, O. et al. (2009) Annu. Rev. Med. 60:233-45, incorporated herein by reference). In certain embodiments, the fibrotic condition involves excessive collagen mRNA production and deposition. In certain embodiments, the fibrotic condition is caused, at least in part, by injury, e.g., chronic injury (e.g., an insult, a wound, a toxin, a disease). In certain embodiments, the fibrotic condition is associated with an inflammatory, an autoimmune or a connective tissue disorder. For example, chronic inflammation in a tissue can lead to fibrosis in that tissue. Exemplary fibrotic tissues include, but are not limited to, biliary tissue, liver tissue, lung tissue, heart tissue, vascular tissue, kidney tissue, skin tissue, gut tissue, peritoneal tissue, bone marrow, and the like. In certain embodiments, the tissue is epithelial tissue.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. By way of example, in the context of fibrotic conditions, “treating,” “treat,” and “treatment” as used herein, refers to partially or completely inhibiting or reducing the fibrotic condition which the subject is suffering. In one embodiment, this term refers to an action that occurs while a patient is suffering from, or is diagnosed with, the fibrotic condition, which reduces the severity of the condition, or retards or slows the progression of the condition. Treatment need not result in a complete cure of the condition; partial inhibition or reduction of the fibrotic condition is encompassed by this term.

“Therapeutically effective amount,” as used herein, refers to a minimal amount or concentration of an ERβ agonist that, when administered alone or in combination, is sufficient to provide a therapeutic benefit in the treatment of the condition, or to delay or minimize one or more symptoms associated with the condition. The term “therapeutically effective amount” can encompass an amount that improves overall therapy, reduces or avoids symptoms or causes of the condition, or enhances the therapeutic efficacy of another therapeutic agent. The therapeutic amount need not result in a complete cure of the condition; partial inhibition or reduction of the fibrotic condition is encompassed by this term.

As used herein, unless otherwise specified, the terms “prevent,” “preventing” and “prevention” refers to an action that occurs before the subject begins to suffer from the condition, or relapse of such condition. The prevention need not result in a complete prevention of the condition; partial prevention or reduction of the fibrotic condition is encompassed by this term.

As used herein, unless otherwise specified, a “prophylactically effective amount” of an ERβ that, when administered alone or in combination, prevent the condition, or one or more symptoms associated with the condition, or prevent its recurrence. The term “prophylactically effective amount” can encompass an amount that improves overall prophylaxis or enhances the prophylactic efficacy of another prophylactic agent. The prophylactic amount need not result in a complete prevention of the condition; partial prevention or reduction of the fibrotic condition is encompassed by this term.

The term “anticancer” refers to the ability to treat or control cellular proliferation and/or tumor growth at any concentration.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

### Chemical Definitions

Terms used herein will have their customary meaning in the art unless specified otherwise. The organic moieties mentioned when defining variable positions within the general formulae described herein (e.g., the term “halogen”) are collective terms for the individual substituents encompassed by the organic moiety. The prefix Cn-Cm preceding a group or moiety indicates, in each case, the possible number of carbon atoms in the group or moiety that follows.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, heteroatoms present in a compound or moiety, such as nitrogen, can have hydrogen substituents and/or any permissible substituents of organic compounds described herein which satisfy the valency of the heteroatom. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound (e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, and 1-ethyl-2-methyl-propyl. Alkyl substituents may be unsubstituted or substituted with one or more chemical moieties. The alkyl group can be substituted with one or more groups including, but not limited to, hydroxy, halogen, acyl, alkyl, alkoxy, alkenyl, alkynyl, aryl, heteroaryl, acyl, aldehyde, amino, carboxylic acid, ester, ether, ketone, nitro, silyl, sulfo-oxo, sulfonyl, sulfone, sulfoxide, or thiol, as described below, provided that the substituents are sterically compatible and the rules of chemical bonding and strain energy are satisfied. The alkyl group can also include one or more heteroatoms (e.g., from one to three heteroatoms) incorporated within the hydrocarbon moiety. Examples of heteroatoms include, but are not limited to, nitrogen, oxygen, sulfur, and phosphorus.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure-CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 20 carbon atoms. Aryl groups can include a single ring or multiple condensed rings. In some embodiments, aryl groups include C₆-C₁₀ aryl groups. Examples of aryl groups include, but are not limited to, phenyl, biphenyl, naphthyl, tetrahydronaphthyl, phenylcyclopropyl, and indanyl. In some embodiments, the aryl group can be a phenyl, indanyl or naphthyl group. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, cycloalkyl, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl has 5-10 ring atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, and 7-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl has 4-10, 4-7 or 4-6 ring atoms with 1 or 2 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “acyl” as used herein is represented by the formula —C(O)Z¹ where Z¹ can be a hydrogen, hydroxyl, alkoxy, alkyl, halogenated alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a short hand notation for C═O.

As used herein, the term “alkoxy” refers to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H.

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

As used herein, Me refers to a methyl group; OMe refers to a methoxy group; and i-Pr refers to an isopropyl group.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

Reference will now be made in detail to specific aspects of the disclosed materials, compounds, compositions, articles, and methods, examples of which are illustrated in the accompanying Examples and Figures.

Carboranes and Carborane Analogs

Dicarba-closo-dodecaborane (also referred to herein as “carborane”) is an icosahedral cluster containing two carbon atoms and ten boron atoms in which both atoms are hexacoordinated. In carboranes, depending on the position of the carbon atoms in the cluster, 3 kinds of isomers exist, i.e., 1,2-dicarba-closo-dodecaborane (ortho-carborane), 1,7-dicarba-closo-dodecaborane (meta-carborane), and 1,12-dicarba-closo-dodecaborane (para-carborane). These structures are unique among boron compounds, as they can have high thermal stabilities and hydrophobicities, for example, comparable to hydrocarbons.

Carboranes can be used, for example, in ¹⁰Boron-Neutron Capture Therapy (BNCT). BNCT has been developed as a therapy for glioma and melanoma. When ¹⁰B is irradiated with thermal neutron (slow neutron), and α ray with 2.4 MeV energy is emitted and the atom decomposed to ⁷Li and ⁴He. The range of α ray is about 10 μm, which corresponds to the diameter of cells Therefore, effects are expected that only cells in which ¹⁰B atoms are uptaken are destroyed and other cells are not damaged. For the development of BNCT, it is important to have cancer cells selectively uptake ¹⁰B atoms in a concentration capable of destroying cells with neutron radiation. For that purpose, other-carborane skeleton has been utilized which has been utilized which has low toxicity and a high ¹⁰B content, and is easy to be synthesized. Moreover, nucleic acid precursors, amino acids, and porphyrins which contain ortho-carboranes have been synthesized and subjected to evaluation.

Carborane-based ERβ agonists are described, for example, in U.S. Pat. No. 6,838,574 to Endo and U.S. Patent Application Publication No. 2018/0264017 to Tjarks et al., each of which is hereby incorporated by reference in its entirety.

In some embodiments, the carborane can be defined by Formula I below

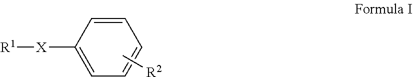

wherein

R¹ represents a dicarba-closo-dodecaboran-yl group which may have one or more substituents selected from the group consisting of an alkyl group, an alkenyl group, a carboxyl group, an alkoxycarbonyl group, an amino group, a hydroxyl group, a hydroxyalkyl group, a mono or di-alkylcarbamoyl-substituted alkyl group, an alkanoyl group, an aryl group, and an aralkyl group, each of which may be substituted or unsubstituted;

R² represents a carboxyl group, an alkoxycarbonyl group, or a hydroxyl group;

X represents a single bond, or a linking group selected from the group consisting of groups represented by the following formulas:

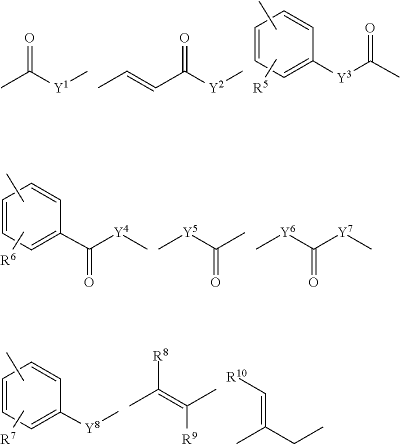

wherein Y¹, Y², Y³, Y⁴ Y⁵, Y⁶, and Y⁷ independently represent an oxygen atom or —(R³ wherein R³ represents hydrogen atom or an alkyl group; Y⁸ represents an oxygen atom, —N(R⁴)— wherein R⁴ represents hydrogen atom or an alkyl group, —CO—, —CH₂—, or —C(═CH²)—; R⁵, R⁶, and R⁷ independently represent hydrogen or one or more substituents on the phenyl group; R⁸ represents an alkyl group or an aryl group which may be substituted; R⁹ represents an alkyl group; and R¹⁰ represents a substituted or unsubstituted aryl group.

In some embodiments, the carborane can be defined by Formula II, or a pharmaceutically acceptable salts thereof:

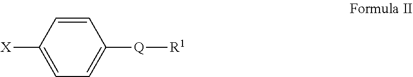

wherein

Q is a substituted or unsubstituted dicarba-closo-dodecaborane cluster, and

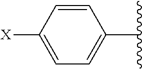

and R¹ are attached to Q in a para configuration;

X is OH, NHR², SH, or S(O)(O)NHR²;

R¹ is substituted or unsubstituted C₄-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteraryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, or NR³R⁴;

R² is H, OH, halogen, or substituted or unsubstituted C₁-C₄ alkyl;

R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl;

with the proviso that when X is OH, R¹ is not (CH₂)₅CH(CH₃)₂ or NH₂.

In some examples of Formula II, the carborane cluster can include a heteroatom. In some examples of Formula II, the carborane cluster can include an isotopically labeled atom (i.e., a radiolabeled atom). In some examples of Formula II, the carborane cluster can include an isotopically labeled Boron atom (e.g., ¹⁰B).

In some examples of Formula II, Q can be:

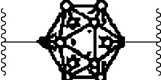

wherein

● is a carbon atom or a boron atom; and

∘ is C—H, C-halogen, C-alkyl, C—OH, C—NH₂, B—H, B-halogen, B-alkyl, B—OH, or B—NH₂.

In some examples of Formula II, X is OH.

In some examples of Formula II, R¹ is a substituted or unsubstituted C₆-C₁₀ alkyl. In some examples of Formula II, R¹ is a C₆-C₁₀ hydroxyalkyl. In some examples of Formula II, R¹ is a substituted or unsubstituted C₃-C₁₆ alkylaryl. In some examples of Formula II, R¹ is a C₃-C₁₆ hydroxyalkylaryl. In some examples of Formula II, R¹ is a substituted or unsubstituted C₅-C₁₀ acyl. In some examples of Formula II, R¹ is a substituted or unsubstituted branched C₄-C₁₀ alkyl. In some examples of Formula II, R¹ is a branched C₄-C₁₀ hydroxyalkyl.

In some examples of Formula II, the compounds can be of Formula III, or a pharmaceutically acceptable salt thereof:

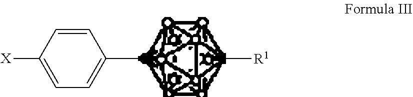

wherein

● is a carbon atom;

∘ is B—H, B-halogen, B-alkyl, B—OH, or B—NH₂;

X is OH, NHR², SH, or S(O)(O)NHR²;

R¹ is substituted or unsubstituted C₄-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, or NR³R⁴;

R² is H, OH, halogen, or substituted or unsubstituted C₁-C₄ alkyl; and

R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl;

with the proviso that when X is OH, R¹ is not (CH₂)SCH(CH₃)₂ or NH₂.

In some examples of Formula III, the carborane cluster can include a heteroatom.

In some examples of Formula III, the carborane cluster can include an isotopically labeled atom (i.e., a radiolabeled atom). In some examples of Formula III, the carborane cluster can include an isotopically labeled Boron atom (e.g., ¹⁰B).

In some examples of Formula III, X is OH.

In some examples of Formula III, R¹ is a substituted or unsubstituted C₆-C₁₀ alkyl. In some examples of Formula III, R¹ is a C₆-C₁₀ hydroxyalkyl. In some examples of Formula III, R¹ is a substituted or unsubstituted C₃-C₁₆ alkylaryl. In some examples of Formula III, R¹ is a C₃-C₁₆ hydroxyalkylaryl. In some examples of Formula III, R¹ is a substituted or unsubstituted C₅-C₁₀ acyl. In some examples of Formula III, R¹ is a substituted or unsubstituted branched C₄-C₁₀ alkyl. In some examples of Formula III, R¹ is a branched C₄-C₁₀ hydroxyalkyl.

In some examples of Formula III, the compounds can be of Formula IV, or a pharmaceutically acceptable salt thereof:

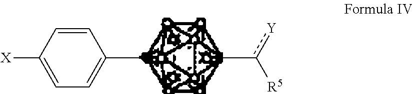

wherein

● is a carbon atom;

∘ is B—H, B-halogen, B-alkyl, B—OH, or B—NH₂;

the dotted line to Y indicates that the bond can be a single bond or a double bond, as valence permits;

X is OH, NHR², SH, or S(O)(O)NHR²;

Y is O, OR^{2′}, NHR², SH, or S(O)(O)NHR²;

R⁵ is substituted or unsubstituted C₂-C₁₉ alkyl, substituted or unsubstituted C₂-C₁₉ alkenyl, substituted or unsubstituted C₂-C₁₉ alkynyl, substituted or unsubstituted C₂-C₁₉ alkylaryl, substituted or unsubstituted C₂-C₁₉ alkylheteroaryl, substituted or unsubstituted C₃-C₁₉ alkylcycloalkyl, substituted or unsubstituted C₃-C₁₉ alkylheterocycloalkyl, or NR³R⁴;

R² is H, OH, halogen, or substituted or unsubstituted C₁-C₄ alkyl;

R^{2′} is H or substituted or unsubstituted C₁-C₄ alkyl; and

R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl.

In some examples of Formula IV, the carborane cluster can include a heteroatom. In some examples of Formula IV, the carborane cluster can include an isotopically labeled atom (i.e., a radiolabeled atom). In some examples of Formula IV, the carborane cluster can include an isotopically labeled Boron atom (e.g., ¹⁰B).

In some examples of Formula IV, X is OH.

In some examples of Formula IV, Y is OH. In some examples of Formula IV, Y is O.

In some examples of Formula IV, R⁵ is a substituted or unsubstituted C₃-C₉ alkyl. In some examples of Formula IV, R⁵ is a substituted or unsubstituted C₆-C₉ alkyl. In some examples of Formula IV, R⁵ is a substituted or unsubstituted C₂-C₁₅ alkylaryl. In some examples of Formula IV, R⁵ is a substituted or unsubstituted branched C₂-C₉ alkyl.

Also disclosed herein are compounds of Formula V, and pharmaceutically acceptable salts thereof:

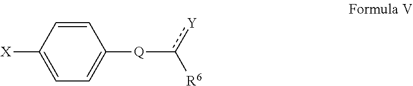

wherein

Q is a substituted or unsubstituted dicarba-closo-dodecaborane cluster, and

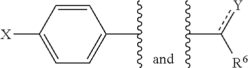

are attached to Q in a para configuration;

the dotted line to Y indicates that the bond can be a single bond or a double bond, as valence permits;

X is OH, NHR², SH, or S(O)(O)NHR²;

Y is O, OR^{2′}, NHR², SH, or S(O)(O)NHR²;

R⁶ is substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₂-C₂₀ alkylheteroaryl, substituted or unsubstituted C₄-C₂₀ alkylcycloalkyl, substituted or unsubstituted C₄-C₂₀ alkylheterocycloalkyl, or NR³R⁴;

R² is H, OH, halogen, or substituted or unsubstituted C₁-C₄ alkyl;

R^{2′} is H or substituted or unsubstituted C₁-C₄ alkyl; and

R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl;

with the proviso that when X is OH, R⁶ is not CH₂OH, CH(CH₃)OH, CH₂CH₂OH, CH₂CH₂CH₂OH, (CH₂)SCH(CH₃)₂, or NH₂.

In some examples of Formula V, the carborane cluster can include a heteroatom. In some examples of Formula V, the carborane cluster can include an isotopically labeled atom (i.e., a radiolabeled atom). In some examples of Formula V, the carborane cluster can include an isotopically labeled Boron atom (e.g., ¹⁰B).

In some examples of Formula V, Q can be

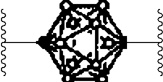

wherein

● is a carbon atom or a boron atom; and

∘ is C—H, C-halogen, C-alkyl, C—OH, C—NH₂, B—H, B-halogen, B-alkyl, B—OH, or B—NH₂.

In some examples of Formula V, X is OH.

In some examples of Formula V, Y is OH. In some examples of Formula V, Y is O.

In some examples of Formula V, R⁶ is a substituted or unsubstituted C₆-C₁₀ alkyl. In some examples of Formula V, R⁶ is a substituted or unsubstituted C₂-C₁₅ alkylaryl. In some examples of Formula V, R⁶ is a substituted or unsubstituted branched C₃-C₁₀ alkyl.

In some examples of Formula V, the compounds can be of Formula VI, or a pharmaceutically acceptable salt thereof:

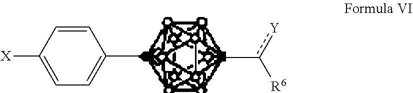

wherein

● is a carbon atom;

∘ is B—H, B-halogen, B-alkyl, B—OH, or B—NH₂;

the dotted line to Y indicates that the bond can be a single bond or a double bond, as valence permits;

X is OH, NHR², SH, or S(O)(O)NHR²;

Y is O, OR^{2′}, NHR², SH, or S(O)(O)NHR²;

R⁶ is substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₂-C₂₀ alkylheteroaryl, substituted or unsubstituted C₄-C₂₀ alkylcycloalkyl, substituted or unsubstituted C₄-C₂₀ alkylheterocycloalkyl, or NR³R⁴;

R² is H, OH, halogen, or substituted or unsubstituted C₁-C₄ alkyl;

R^{2′} is H or substituted or unsubstituted C₁-C₄ alkyl; and

R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl;

with the proviso that when X is OH, R⁶ is not CH₂OH, CH(CH₃)OH, CH₂CH₂OH, CH₂CH₂CH₂OH, (CH₂)SCH(CH₃)₂, or NH₂.

In some examples of Formula VI, the carborane cluster can include a heteroatom. In some examples of Formula VI, the carborane cluster can include an isotopically labeled atom (i.e., a radiolabeled atom). In some examples of Formula VI, the carborane cluster can include an isotopically labeled Boron atom (e.g., ¹⁰B).

In some examples of Formula VI, X is OH.

In some examples of Formula VI, Y is OH. In some examples of Formula VI, Y is O.

In some examples of Formula VI, R⁶ is a substituted or unsubstituted C₆-C₁₀ alkyl. In some examples of Formula VI, R⁶ is a substituted or unsubstituted C₂-C₁₅ alkylaryl. In some examples of Formula VI, R⁶ is a substituted or unsubstituted branched C₃-C₁₀ alkyl.

Also disclosed herein are compounds of Formula VII, and pharmaceutically acceptable salts thereof:

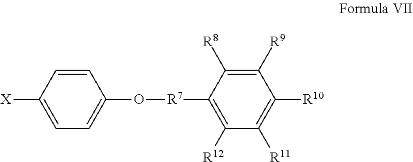

wherein

Q is a substituted or unsubstituted dicarba-closo-dodecaborane cluster, and

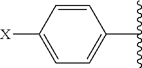

and R⁷ are attached to Q in a para configuration;

X is OH, NHR², SH, or S(O)(O)NHR²;

R⁷ is substituted or unsubstituted C₁-C₁₄ alkyl, substituted or unsubstituted C₂-C₁₄ alkenyl, substituted or unsubstituted C₂-C₁₄ alkynyl, substituted or unsubstituted C₁-C₁₄ acyl, or NR³R⁴;

R⁸, R⁹, R¹⁰, R¹¹, and R¹² are independently H, OH, halogen, substituted or unsubstituted C₁-C₂₀ alkyl, sub substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, substituted or unsubstituted C₁-C₂₀ acyl, or NR³R⁴, or wherein, as valence permits, R⁸ and R⁹, R⁹ and R¹⁰, R¹⁰ and R¹¹, or R¹¹ and R¹², together with the atoms to which they are attached, form a 3-10 membered substituted or unsubstituted cyclic moiety optionally including from 1 to 3 heteroatoms;

R² is H, OH, halogen, or substituted or unsubstituted C₁-C₄ alkyl; and

R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl.

In some examples of Formula VII, the carborane cluster can include a heteroatom. In some examples of Formula VII, the carborane cluster can include an isotopically labeled atom (i.e., a radio labeled atom). In some examples of Formula VII, the carborane cluster can include an isotopically labeled Boron atom (e.g., ¹⁰B).

In some examples of Formula VII, Q can be

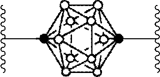

wherein

● is a carbon atom or a boron atom; and

∘ is C—H, C-halogen, C-alkyl, C—OH, C—NH₂, B—H, B-halogen, B-alkyl, B—OH, or B—NH₂.

In some examples of Formula VII, X is OH.

In some examples of Formula VII, R⁷ is a substituted or unsubstituted C₁-C₇ alkyl. In some examples of Formula VII, R⁷ is a C₁-C₇ hydroxyalkyl.

In some examples of Formula VII, R⁸-R¹² are independently H, OH, halogen, or substituted or unsubstituted C₁-C₄ alkyl, or wherein, as valence permits, R⁸ and R⁹, R⁹ and R¹⁰, R¹⁰ and R¹¹, or R¹¹ and R¹², together with the atoms to which they are attached, form a 3-10 membered substituted or unsubstituted cyclic moiety optionally including from 1 to 3 heteroatoms. In some examples of Formula VII, R⁸—R¹² are each H. In some examples of Formula VII, R⁸, R¹⁰, and R¹² are each H, and R⁹ and R¹⁰, together with the atoms to which they are attached, form a substituted or unsubstituted 5-7 membered cyclic moiety.

In some examples of Formula VII, the compounds can be of Formula VIII, or a pharmaceutically acceptable salt thereof:

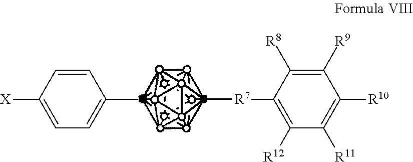

wherein

● is a carbon atom;

∘ is B—H, B-halogen, B-alkyl, B—OH, or B—NH₂;

X is OH, NHR², SH, or S(O)(O)NHR²;

R⁷ is substituted or unsubstituted C₁-C₁₄ alkyl, substituted or unsubstituted C₂-C₁₄ alkenyl, substituted or unsubstituted C₂-C₁₄ alkynyl, substituted or unsubstituted C₁-C₁₄ acyl, or NR³R⁴;

R⁸, R⁹, R¹⁰, R¹¹, and R¹² are independently H, OH, halogen, substituted or unsubstituted C₁-C₂₀ alkyl, sub substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, substituted or unsubstituted C₁-C₂₀ acyl, or NR³R⁴, or wherein, as valence permits, R⁸ and R⁹, R⁹ and R¹⁰, R¹⁰ and R¹¹, or R¹¹ and R¹², together with the atoms to which they are attached, form a 3-10 membered substituted or unsubstituted cyclic moiety optionally including from 1 to 3 heteroatoms;

R² is H, OH, halogen, or substituted or unsubstituted C₁-C₄ alkyl; and

R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl.

In some examples of Formula VIII, the carborane cluster can include a heteroatom. In some examples of Formula VIII, the carborane cluster can include an isotopically labeled atom (i.e., a radiolabeled atom). In some examples of Formula VIII, the carborane cluster can include an isotopically labeled Boron atom (e.g., ¹⁰B).

In some examples of Formula VIII, X is OH.

In some examples of Formula VIII, R⁷ is a substituted or unsubstituted C₁-C₇ alkyl. In some examples of Formula VIII, R⁷ is a C₁-C₇ hydroxyalkyl.

In some examples of Formula VIII, R⁸-R¹² are independently H, OH, halogen, or substituted or unsubstituted C₁-C₄ alkyl, or wherein, as valence permits, R⁸ and R⁹, R⁹ and R¹⁰, R¹⁰ and R¹¹, or R¹¹ and R¹², together with the atoms to which they are attached, form a 3-10 membered substituted or unsubstituted cyclic moiety optionally including from 1 to 3 heteroatoms. In some examples of Formula VIII, R⁸-R¹² are each H. In some examples of Formula VIII, R⁸, R¹⁰, and R¹² are each H, and R⁹ and R¹², together with the atoms to which they are attached, form a substituted or unsubstituted 5-7 membered cyclic moiety.

Also disclosed herein are compounds of Formula IX, and pharmaceutically acceptable salts thereof:

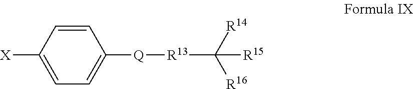

wherein

Q is a substituted or unsubstituted dicarba-closo-dodecaborane cluster, and

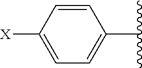

and R³ are attached to Q in a para configuration;

X is OH, NHR², SH, or S(O)(O)NHR²;

R¹³ is substituted or unsubstituted C₁-C₁₉ alkyl, substituted or unsubstituted C₂-C₁₉ alkenyl, substituted or unsubstituted C₂-C₁₉ alkynyl, or substituted or unsubstituted C₁-C₂₀ acyl; and

R¹⁴, R¹⁵, and R¹⁶ are independently hydrogen, halogen, hydroxyl, substituted or unsubstituted C₁-C₁₈ alkyl, substituted or unsubstituted C₂-C₁₈ alkenyl, substituted or unsubstituted C₁-C₁₈ alkynyl, substituted or unsubstituted C₂-C₁₈ aryl, substituted or unsubstituted C₃-C₁₈ cycloalkyl, substituted or unsubstituted C₁-C₂₀ acyl, or NR³R⁴, or wherein, as valence permits, R¹⁴ and R¹⁵, R¹⁴ and R¹⁶, or R¹⁵ and R¹⁶, together with the atoms to which they are attached, for a 3-10 membered substituted or unsubstituted cyclic moiety optionally including from 1 to 3 heteroatoms,

with the proviso that at least two of R¹⁴, R¹⁵ and R¹⁶ are not hydrogen, halogen, or hydroxyl; and

with the proviso that when X is OH and R¹³ is a C₅ alkyl, R¹⁴, R¹⁵, and R¹⁶ are not H, methyl, and methyl.

In some examples of Formula IX, the carborane cluster can include a heteroatom. In some examples of Formula IX, the carborane cluster can include an isotopically labeled atom (i.e., a radio labeled atom). In some examples of Formula IX, the carborane cluster can include an isotopically labeled Boron atom (e.g., ¹⁰B). In some examples of Formula IX, Q is

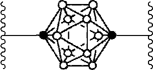

wherein

● is a carbon atom or a boron atom; and

∘ is C—H, C-halogen, C-alkyl, C—OH, C—NH₂, B—H, B-halogen, B-alkyl, B—OH, or B—NH₂.

In some examples of Formula IX, X is OH.

In some examples of Formula IX, R¹³ is a substituted or unsubstituted C₄-C₈ alkyl. In some examples of Formula IX, R¹³ is a C₄-C₈ hydroxyalkyl.

In some examples of Formula IX, R¹⁴-R¹⁶ are independently hydrogen, halogen, hydroxyl, substituted or unsubstituted C₁-C₄ alkyl, with the proviso that at least two of R¹⁴, R¹⁵ and R¹⁶ are not hydrogen, halogen, or hydroxyl; and with the proviso that when X is OH and R¹³ is a C₅ alkyl, R¹⁴, R¹⁵, and R¹⁶ are not H, methyl, and methyl.

In some examples of Formula IX, the compounds can be of Formula X, or a pharmaceutically acceptable salt thereof:

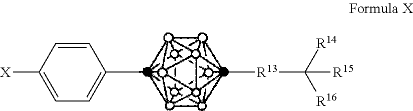

wherein

● is a carbon atom;

∘ is B—H, B-halogen, B-alkyl, B—OH, or B—NH₂;

X is OH, NHR², SH, or S(O)(O)NHR²;

R¹³ is substituted or unsubstituted C₁-C₁₉ alkyl, substituted or unsubstituted C₂-C₁₉ alkenyl, substituted or unsubstituted C₂-C₁₉ alkynyl, or substituted or unsubstituted C₁-C₂₀ acyl; and

R¹⁴, R¹⁵, and R¹⁶ are independently hydrogen, halogen, hydroxyl, substituted or unsubstituted C₁-C₁₈ alkyl, substituted or unsubstituted C₂-C₁₈ alkenyl, substituted or unsubstituted C₁-C₁₈ alkynyl, substituted or unsubstituted C₂-C₁₈ aryl, substituted or unsubstituted C₃-C₁₈ cycloalkyl, substituted or unsubstituted C₁-C₂₀ acyl, or NR³R⁴, or wherein, as valence permits, R¹⁴ and R¹⁵, R¹⁴ and R¹⁶, or R¹⁵ and R¹⁶, together with the atoms to which they are attached, for a 3-10 membered substituted or unsubstituted cyclic moiety optionally including from 1 to 3 heteroatoms,

with the proviso that at least two of R¹⁴, R¹⁵ and R¹⁶ are not hydrogen, halogen, or hydroxyl; and

with the proviso that when X is OH and R³ is a C₅ alkyl, R¹, R⁵, and R¹⁶ are not H, methyl, and methyl.

In some examples of Formula X, the carborane cluster can include a heteroatom. In some examples of Formula X, the carborane cluster can include an isotopically labeled atom (i.e., a radio labeled atom). In some examples of Formula X, the carborane cluster can include an isotopically labeled Boron atom (e.g., ¹⁰B).

In some examples of Formula X, X is OH.

In some examples of Formula X, R¹³ is a substituted or unsubstituted C₄-C₈ alkyl. In some examples of Formula X, R¹³ is a C₄-C₈ hydroxyalkyl.

In some examples of Formula X, R¹⁴-R¹⁶ are independently hydrogen, halogen, hydroxyl, substituted or unsubstituted C₁-C₄ alkyl, with the proviso that at least two of R¹⁴, R¹⁵ and R¹⁶ are not hydrogen, halogen, or hydroxyl; and with the proviso that when X is OH and R¹³ is a C₅ alkyl, R¹⁴, R¹⁵, and R¹⁶ are not H, methyl, and methyl.

In some examples, the compounds can be selected from the group consisting of:

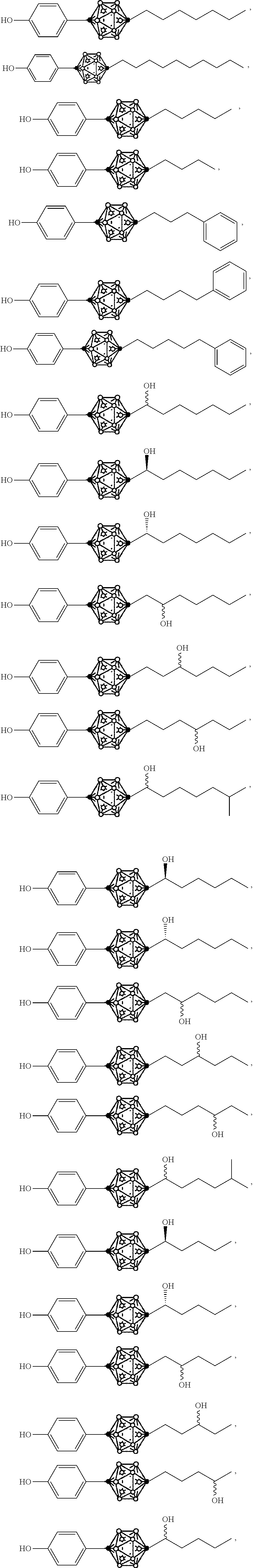

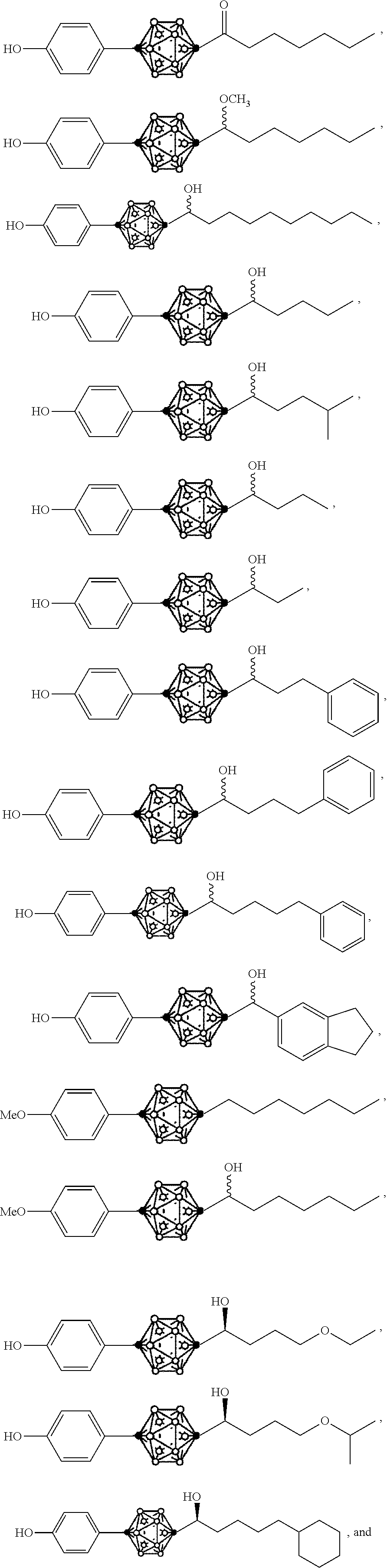

pharmaceutically acceptable salts thereof. In some examples, the carborane cluster can include a heteroatom.

Also disclosed herein are compounds of Formula XI, and pharmaceutically acceptable salts thereof:

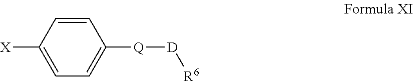

wherein

Q is a substituted or unsubstituted dicarba-closo-dodecaborane cluster;

D is —S—, —S(O)—, —S(O)(O)—, —S(O)(NH)—, —P(O)(OH)O—, —P(O)(OH)NH—, or —O—;

X is OH, NHR², SH, or S(O)(O)NHR²;

R⁶ is substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₂-C₂₀ alkylheterlaryl, substituted or unsubstituted C₄-C₂₀ alkylcycloalkyl, or substituted or unsubstituted C₄-C₂₀ alkylheterocycloalkyl; and

R² is H, OH, halogen, or substituted or unsubstituted C₁-C₄ alkyl.

In some examples of Formula XI

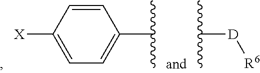

are attached to Q in a para configuration.

In some examples of Formula XI, the carborane cluster can include a heteroatom. In some examples of Formula XI, the carborane cluster can include an isotopically labeled atom (i.e., a radiolabeled atom). In some examples of Formula XI, the carborane cluster can include an isotopically labeled Boron atom (e.g., ¹⁰B).

In some examples of Formula XI, Q can be

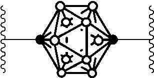

wherein

● is a carbon atom or a boron atom; and

∘ is C—H, C-halogen, C-alkyl, C—OH, C—NH₂, B—H, B-halogen, B-alkyl, B—OH, or B—NH₂.

In some examples of Formula XI, X is OH.

In some examples of Formula XI, R⁶ is a substituted or unsubstituted C₆-C₁₀ alkyl. In some examples of Formula XI, R⁶ is a substituted or unsubstituted C₂-C₁₅ alkylaryl. In some examples of Formula XI, R⁶ is a substituted or unsubstituted branched C₃-C₁₀ alkyl.

In some examples, the compounds can be selected from the group consisting of:

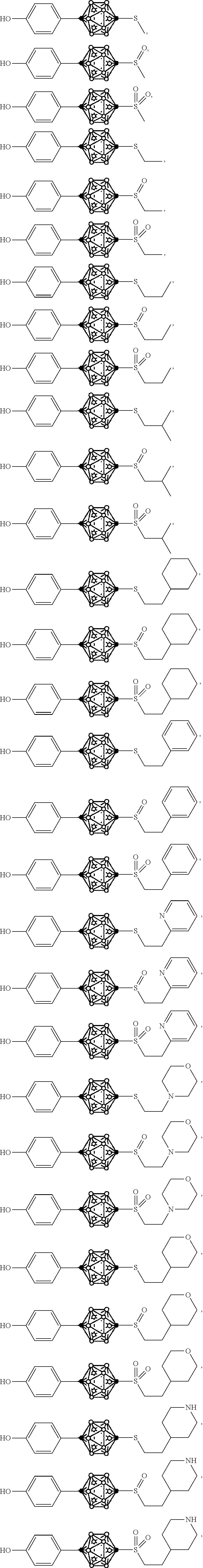

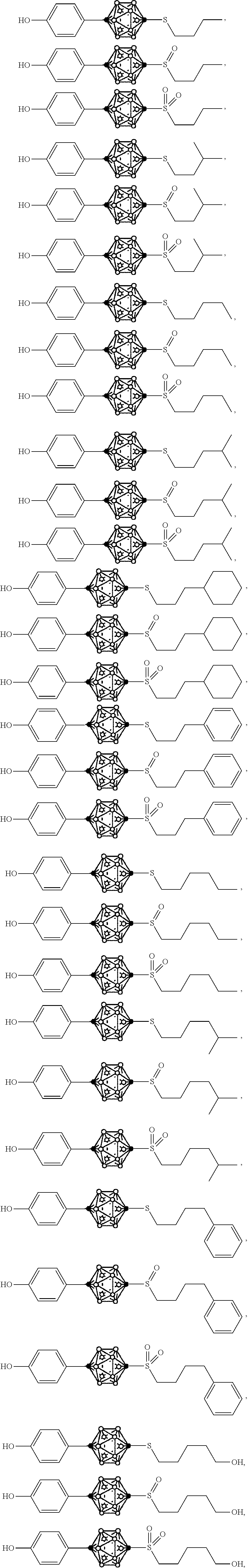

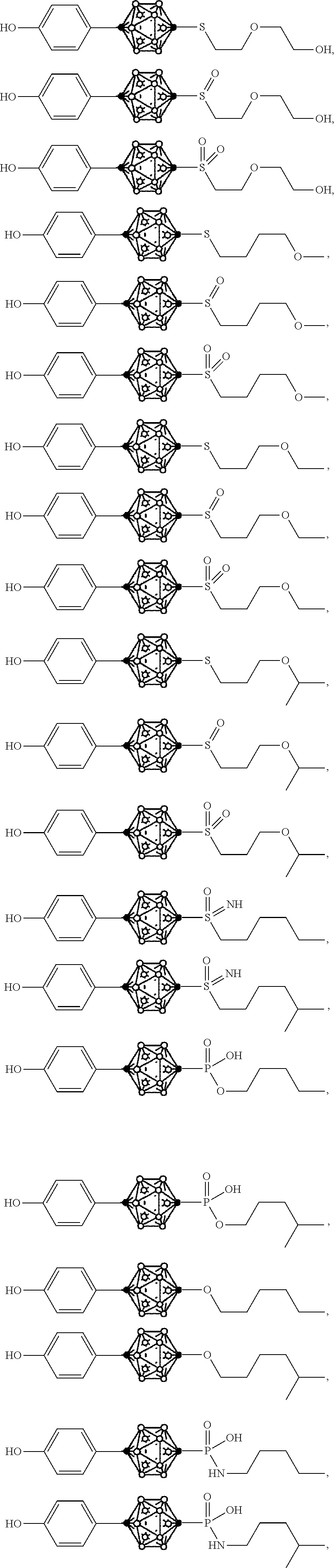

pharmaceutically acceptable salts thereof. In some examples, the carborane cluster can include a heteroatom.

In some embodiments, the carborane can be defined by Formula XII, or a pharmaceutically acceptable salt thereof:

A-Q-R¹ Formula XII

wherein Q is a substituted or unsubstituted dicarba-closo-dodecaborane cluster, and A and R¹ are attached to Q in a para configuration; A is a substituted or unsubstituted heteroaryl ring; R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)N R³R⁴, —S(O)—R³, —S(O₂)—R³, substituted or unsubstituted C₂-C₂₀ heteroalkyl, or NR³R⁴; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some embodiments, Q is

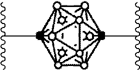

wherein ● is a carbon atom or a boron atom; and ∘ is C—H, C-halogen, C-alkyl, C—OH, C—NH₂, B—H, B-halogen, B-alkyl, B—OH, or B—NH₂.

In some embodiments, A can be a five-membered substituted or unsubstituted heteroaryl ring. For example, A can comprise a thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, or 1,3,4-oxadiazolyl ring. In some embodiments, A can be a six-membered substituted or unsubstituted heteroaryl ring. For example, A can comprise a pyridyl, pyrazinyl, pyrimidinyl, triazinyl, or pyridazinyl ring.

In some cases, the compound can be defined by Formula XIIA, or a pharmaceutically acceptable salt thereof:

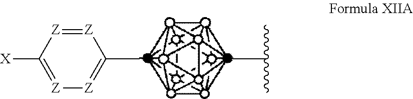

wherein ● is a carbon atom; ∘ is B—H, B-halogen, B-alkyl, B—OH, or B—NH₂; X is OH, NHR², SH, or S(O)(O)NHR²; Z is, individually for each occurrence, N or CH, with the proviso that at least one of Z is N; R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)N R³R⁴, —S(O)—R³, —S(O₂)—R³, substituted or unsubstituted C₂-C₂₀ heteroalkyl, or NR³R⁴; R² is H, OH, halogen, or substituted or unsubstituted C₁-C₄ alkyl; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some cases, one of Z can be N. In some cases, two or more of Z can be N. In some cases, three of Z can be N.

In some embodiments, the compound can be defined by one of the formulae below, or a pharmaceutically acceptable salt thereof:

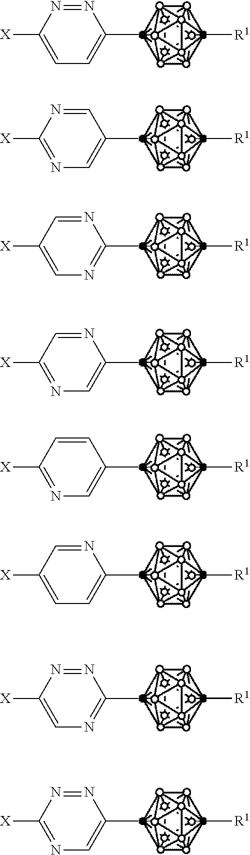

wherein ● is a carbon atom; ∘ is B—H, B-halogen, B-alkyl, B—OH, or B—NH₂; X is OH, NHR², SH, or S(O)(O)NHR²; R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)N R³R⁴, —S(O)—R³, —S(O₂)—R³, substituted or unsubstituted C₂-C₂₀ heteroalkyl, or NR³R⁴; R² is H, OH, halogen, or substituted or unsubstituted C₁-C₄ alkyl; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some embodiments, the compound can be defined by one of Formula XIIB-XIIF, or a pharmaceutically acceptable salt thereof:

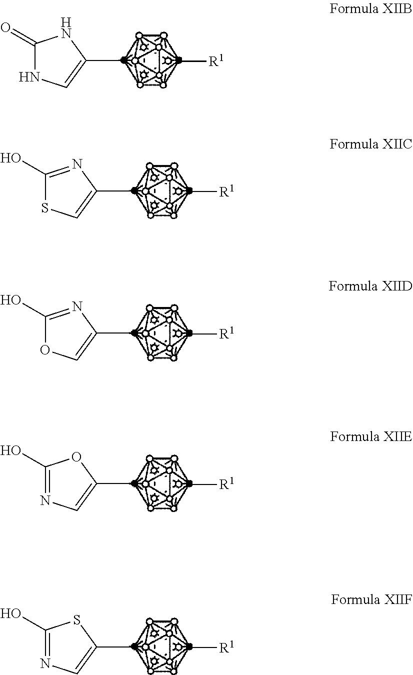

wherein ● is a carbon atom; ∘ is B—H, B-halogen, B-alkyl, B—OH, or B—NH₂; R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)N R³R⁴, —S(O)—R³, —S(O₂)—R³, substituted or unsubstituted C₂-C₂₀ heteroalkyl, or NR³R⁴; R² is H, OH, halogen, or substituted or unsubstituted C₁-C₄ alkyl; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some of the embodiments above, X can be OH.

In some of the embodiments above, R¹ can be a substituted or unsubstituted C₆-C₁₀ alkyl (e.g., a C₆-C₁₀ hydroxyalkyl).

In some of the embodiments above, R¹ can be a substituted or unsubstituted C₃-C₁₆ alkylaryl (e.g., a C₃-C₁₆ hydroxyalkylaryl).

In some of the embodiments above, R¹ can be a substituted or unsubstituted C₅-C₂₀ alkylaryl (e.g., a C₅-C₂₀ hydroxyalkylaryl).

In some of the embodiments above, R¹ can be a substituted or unsubstituted C₅-C₁₀ acyl.

In some of the embodiments above, R¹ can be a substituted or unsubstituted branched C₄-C₁₀ alkyl (e.g., a branched C₄-C₁₀ hydroxyalkyl).

In some embodiments, the compound is defined by a formula below, or a pharmaceutically acceptable salt thereof:

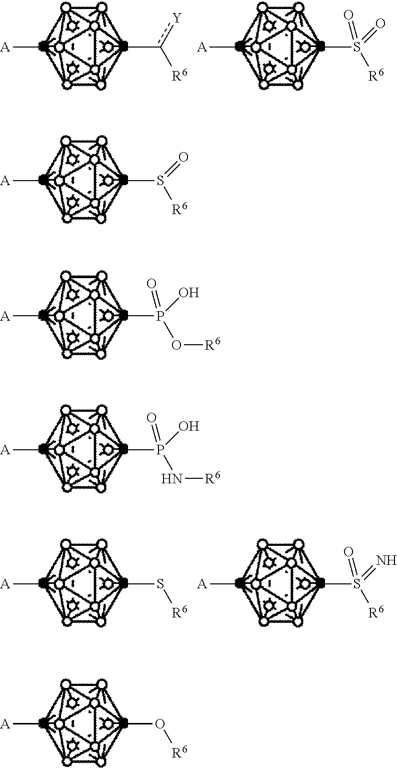

wherein ● is a carbon atom; ∘ is B—H, B-halogen, B-alkyl, B—OH, or B—NH₂; the dotted line to Y indicates that the bond can be a single bond or a double bond, as valence permits; A is a substituted or unsubstituted heteroaryl ring; Y, when present, is O, halogen, OR^{2′}, NHR², SH, or S(O)(O)NHR²; R⁶ is substituted or unsubstituted C₁-C₁₉ alkyl, substituted or unsubstituted C₂-C₁₉ alkenyl, substituted or unsubstituted C₂-C₁₉ alkynyl, substituted or unsubstituted C₂-C₁₉ alkylaryl, substituted or unsubstituted C₂-C₁₉ alkylheteroaryl, substituted or unsubstituted C₄-C₁₉ alkylcycloalkyl, substituted or unsubstituted C₄-C₁₉ alkylheterocycloalkyl, and substituted or unsubstituted C₂-C₂₀ heteroalkyl. or NR³R⁴; R² is H, OH, halogen, or substituted or unsubstituted C₁-C₄ alkyl; R^{2′} is H or substituted or unsubstituted C₁-C₄ alkyl; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some embodiments, A can be a five-membered substituted or unsubstituted heteroaryl ring. For example, A can comprise a thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, or 1,3,4-oxadiazolyl ring. In some embodiments, A can be a six-membered substituted or unsubstituted heteroaryl ring. For example, A can comprise a pyridyl, pyrazinyl, pyrimidinyl, triazinyl, or pyridazinyl ring.

In some of these embodiments, Y is OH. In some of these embodiments, Y is F. In some of these embodiments, Y is O.

In some examples, R⁶ can be a substituted or unsubstituted C₃-C₁₀ alkyl, such as a substituted or unsubstituted C₆-C₉ alkyl.

In some examples, R⁶ can be a substituted or unsubstituted C₂-C₁₅ alkylaryl.

In some examples, R⁶ can be a substituted or unsubstituted branched C₂-C₉ alkyl.

In some examples, R⁶ can be a substituted or unsubstituted C₃-C₁₀ heteroalkyl, such as a substituted or unsubstituted C₆-C₉ heteroalkyl.

Also provided are compounds defined by Formula XIII, or a pharmaceutically acceptable salt thereof:

A-Q-R¹ Formula XIII

wherein Q is a substituted or unsubstituted dicarba-closo-dodecaborane cluster, and A and R¹ are attached to Q in a para configuration; A is a substituted or unsubstituted aryl ring or a substituted or unsubstituted heteroaryl ring; R¹ is substituted or unsubstituted C₂-C₂₀ heteroalkyl, —C(O)N R³R⁴, —S(O)—R³, —S(O₂)—R³, or NR³R⁴; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₂-C₂₀ alkylheteroaryl, substituted or unsubstituted C₄-C₂₀ alkylcycloalkyl, substituted or unsubstituted C₄-C₂₀ alkylheterocycloalkyl, and substituted or unsubstituted C₂-C₂₀ heteroalkyl, with the proviso that when present, at least one of R³ and R⁴ is C₂-C₂₀ heteroalkyl.

In some embodiments, A can comprise a substituted or unsubstituted aryl ring (e.g., a substituted or unsubstituted phenyl ring). In some embodiments, A can be a five-membered substituted or unsubstituted heteroaryl ring. For example, A can comprise a thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, or 1,3,4-oxadiazolyl ring. In some embodiments, A can be a six-membered substituted or unsubstituted heteroaryl ring. For example, A can comprise a pyridyl, pyrazinyl, pyrimidinyl, triazinyl, or pyridazinyl ring.

In some embodiments, Q is

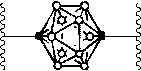

wherein ● is a carbon atom or a boron atom; and ∘ is C—H, C-halogen, C-alkyl, C—OH, C—NH₂, B—H, B-halogen, B-alkyl, B—OH, or B—NH₂.

In some embodiments, the compound can be defined by Formula XIIIA, or a pharmaceutically acceptable salt thereof:

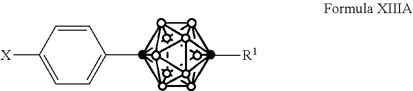

wherein ● is a carbon atom; ∘ is B—H, B-halogen, B-alkyl, B—OH, or B—NH₂; X is OH, NHR², SH, or S(O)(O)NHR²; Z is, individually for each occurrence, N or CH, with the proviso that at least one of Z is N; R¹ is substituted or unsubstituted C₂-C₂₀ heteroalkyl, —C(O)N R³R⁴, —S(O)—R³, —S(O₂)—R³, or NR³R⁴; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₂-C₂₀ alkylheteroaryl, substituted or unsubstituted C₄-C₂₀ alkylcycloalkyl, substituted or unsubstituted C₄-C₂₀ alkylheterocycloalkyl, and substituted or unsubstituted C₂-C₂₀ heteroalkyl, with the proviso that when present, at least one of R³ and R⁴ is C₂-C₂₀ heteroalkyl.

In some of these embodiments, X can be OH.

Also provided are compounds defined by any of the formula below, or a pharmaceutically acceptable salt thereof:

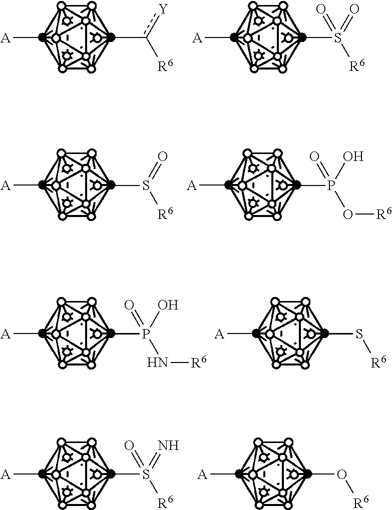

wherein ● is a carbon atom; ∘ is B—H, B-halogen, B-alkyl, B—OH, or B—NH₂; the dotted line to Y indicates that the bond can be a single bond or a double bond, as valence permits; A is a substituted or unsubstituted aryl ring a substituted or unsubstituted heteroaryl ring; Y, when present, is O, halogen, OR^{2′}, NHR², SH, or S(O)(O)NHR²; R⁶ is substituted or unsubstituted C₁-C₁₉ alkyl, substituted or unsubstituted C₂-C₁₉ alkenyl, substituted or unsubstituted C₂-C₁₉ alkynyl, substituted or unsubstituted C₂-C₁₉ alkylaryl, substituted or unsubstituted C₂-C₁₉ alkylheteroaryl, substituted or unsubstituted C₄-C₁₉ alkylcycloalkyl, substituted or unsubstituted C₄-C₁₉ alkylheterocycloalkyl, and substituted or unsubstituted C₂-C₂₀ heteroalkyl. or NR³R⁴; R² is H, OH, halogen, or substituted or unsubstituted C₁-C₄ alkyl; R^{2′} is H or substituted or unsubstituted C₁-C₄ alkyl; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some embodiments, A can comprise a substituted or unsubstituted aryl ring (e.g., a substituted or unsubstituted phenyl ring). In some embodiments, A can be a five-membered substituted or unsubstituted heteroaryl ring. For example, A can comprise a thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, or 1,3,4-oxadiazolyl ring. In some embodiments, A can be a six-membered substituted or unsubstituted heteroaryl ring. For example, A can comprise a pyridyl, pyrazinyl, pyrimidinyl, triazinyl, or pyridazinyl ring.

In some of these embodiments, Y is OH. In some of these embodiments, Y is F. In some of these embodiments, Y is O.

In some examples, R⁶ can be a substituted or unsubstituted C₃-C₁₀ alkyl, such as a substituted or unsubstituted C₆-C₉ alkyl.

In some examples, R⁶ can be a substituted or unsubstituted C₂-C₁₅ alkylaryl.

In some examples, R⁶ can be a substituted or unsubstituted branched C₂-C₉ alkyl.

In some examples, R⁶ can be a substituted or unsubstituted C₃-C₁₀ heteroalkyl, such as a substituted or unsubstituted C₆-C₉ heteroalkyl.

In some examples, the carborane can be selected from the group consisting of:

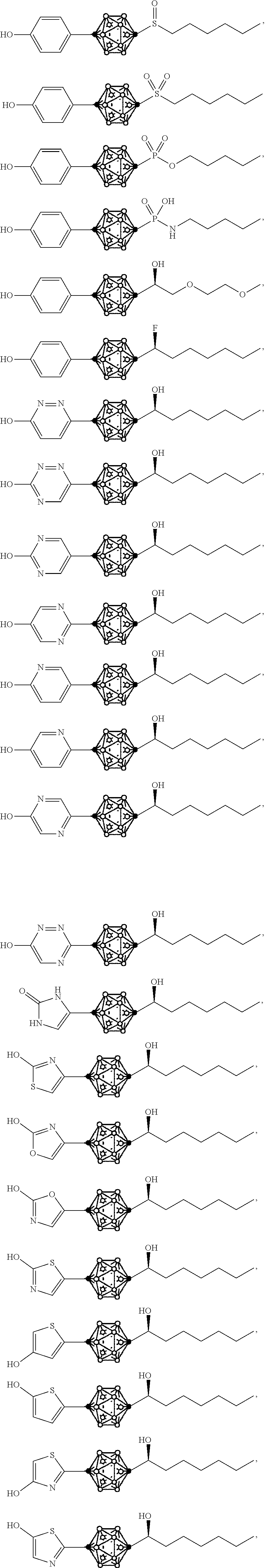

and pharmaceutically acceptable salts thereof. In some examples, the carborane cluster can include a heteroatom.

In some embodiments, the compound can be a carborane analog, such as a dicarba-closo-dodecaborane analog of, for example, the compounds described in WO 2017/049307 to Tjarks et al. The compounds include a spacer group which replaces the carborane moiety in the compounds therein. The resulting compounds can exhibit similar biological activity to the compounds described in WO 2017/049307.

For example, provided herein are compounds defined by Formula XIV, or a pharmaceutically acceptable salt thereof:

A-Q-R¹ Formula XIV

wherein A is a substituted or unsubstituted aryl ring or a substituted or unsubstituted heteroaryl ring; Q is a spacer group chosen from one of the following:

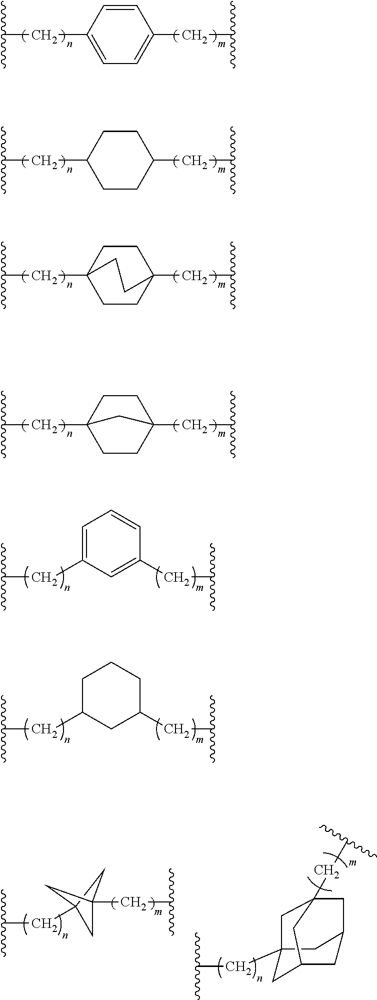

where m and n are each individually 0, 1, 2, or 3; R¹ is substituted or unsubstituted C₄-C₂₀ alkyl, substituted or unsubstituted C₄-C₂₀ heteroalkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)N R³R⁴, or NR³R⁴; and R³ and R⁴ are independently selected from substituted or unsubstituted C₁-C₂₀ alkyl, substituted or unsubstituted C₁-C₂₀ heteroalkyl, substituted or unsubstituted C₂-C₂₀ alkenyl, substituted or unsubstituted C₂-C₂₀ alkynyl, substituted or unsubstituted C₂-C₂₀ alkylaryl, or substituted or unsubstituted C₄-C₂₀ alkylcycloalkyl.

In certain embodiments, Q can be chosen from one of the following:

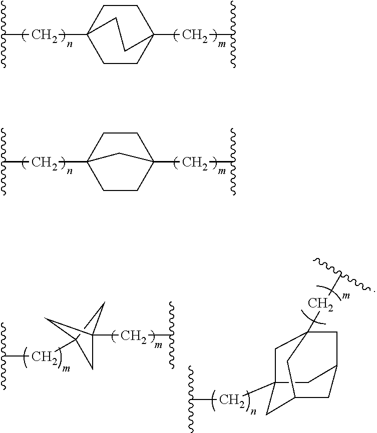

In some embodiments, A can comprise a substituted or unsubstituted aryl ring (e.g., a substituted or unsubstituted phenyl ring). In some embodiments, A can be a five-membered substituted or unsubstituted heteroaryl ring. For example, A can comprise a thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, or 1,3,4-oxadiazolyl ring. In some embodiments, A can be a six-membered substituted or unsubstituted heteroaryl ring. For example, A can comprise a pyridyl, pyrazinyl, pyrimidinyl, triazinyl, or pyridazinyl ring.

In some embodiments, A is

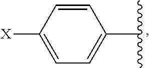

herein X is OH, NHR², SH, or S(O)(O)NHR² and R² is H, OH, halogen, or substituted or unsubstituted C₁-C₄ alkyl. In some of these embodiments, X is OH.

In some embodiments, A is

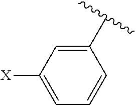

wherein X is OH, NHR², SH, or S(O)(O)NHR² and R² is H, OH, halogen, or substituted or unsubstituted C₁-C₄ alkyl. In some of these embodiments, X is OH.

In some embodiments, A is

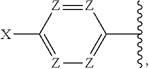

herein Z is, individually for each occurrence, N or CH, with the proviso that at least one of Z is N; X is OH, NHR², SH, or S(O)(O)NHR² and R² is H, OH, halogen, or substituted or unsubstituted C₁-C₄ alkyl. In some of these embodiments, A can be one of the following:

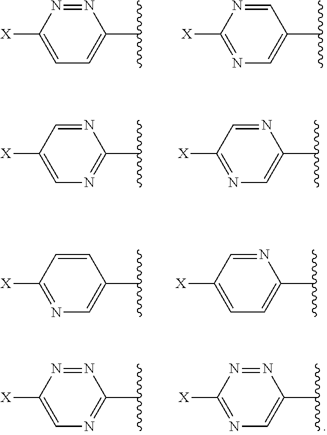

In some of these embodiments, X is OH.

In some embodiments, A is

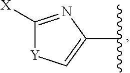

wherein Y is S or O; X is OH, NHR², SH, or S(O)(O)NHR²; and R² is H, OH, halogen, or substituted or unsubstituted C₁-C₄ alkyl. In some of these embodiments, X is OH.

In some embodiments, A is

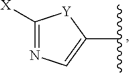

wherein Y is S or O; X is OH, NHR², SH, or S(O)(O)NHR²; and R² is H, OH, halogen, or substituted or unsubstituted C₁-C₄ alkyl. In some of these embodiments, X is OH.

In some embodiments, A is

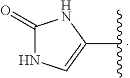 [SMILES: *c1c[nH]c(=O)[nH]1]

In some of the embodiments above, R¹ can be a substituted or unsubstituted C₆-C₁₀ alkyl (e.g., a C₆-C₁₀ hydroxyalkyl).

In some of the embodiments above, R¹ can be a substituted or unsubstituted C₃-C₁₆ alkylaryl (e.g., a C₃-C₁₆ hydroxyalkylaryl).

In some of the embodiments above, R¹ can be a substituted or unsubstituted C₅-C₂₀ alkylaryl (e.g., a C₅-C₂a hydroxyalkylaryl).

In some of the embodiments above, R¹ can be a substituted or unsubstituted C₅-C₁₀ acyl.

In some of the embodiments above, R¹ can be a substituted or unsubstituted branched C₄-C₁₀ alkyl (e.g., a branched C₄-C₁₀ hydroxyalkyl).

In some embodiments, R¹ can comprise one of the following

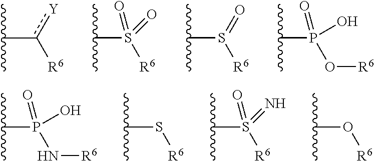

wherein the dotted line to Y indicates that the bond can be a single bond or a double bond, as valence permits; Y, when present, is O, halogen, OR^{2′}, NHR², SH, or S(O)(O)NHR²; R⁶ is substituted or unsubstituted C₁-C₁₉ alkyl, substituted or unsubstituted C₂-C₁₉ alkenyl, substituted or unsubstituted C₂-C₁₉ alkynyl, substituted or unsubstituted C₂-C₁₉ alkylaryl, substituted or unsubstituted C₂-C₁₉ alkylheteroaryl, substituted or unsubstituted C₄-C₁₉ alkylcycloalkyl, substituted or unsubstituted C₄-C₁₉ alkylheterocycloalkyl, and substituted or unsubstituted C₂-C₂₀ heteroalkyl. or NR³R⁴; R² is H, OH, halogen, or substituted or unsubstituted C₁-C₄ alkyl; R^{2′} is H or substituted or unsubstituted C₁-C₄ alkyl; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some embodiments, A can comprise a substituted or unsubstituted aryl ring (e.g., a substituted or unsubstituted phenyl ring). In some embodiments, A can be a five-membered substituted or unsubstituted heteroaryl ring. For example, A can comprise a thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, or 1,3,4-oxadiazolyl ring. In some embodiments, A can be a six-membered substituted or unsubstituted heteroaryl ring. For example, A can comprise a pyridyl, pyrazinyl, pyrimidinyl, triazinyl, or pyridazinyl ring.

In some of these embodiments, Y is OH. In some of these embodiments, Y is F. In some of these embodiments, Y is O.

In some examples, R⁶ can be a substituted or unsubstituted C₃-C₁₀ alkyl, such as a substituted or unsubstituted C₆-C₉ alkyl.

In some examples, R⁶ can be a substituted or unsubstituted C₂-C₁₅ alkylaryl.

In some examples, R⁶ can be a substituted or unsubstituted branched C₂-C₉ alkyl.

In some examples, R⁶ can be a substituted or unsubstituted C₃-C₁₀ heteroalkyl, such as a substituted or unsubstituted C₆-C₉ heteroalkyl.

In some embodiments, the compound can comprise one of the following:

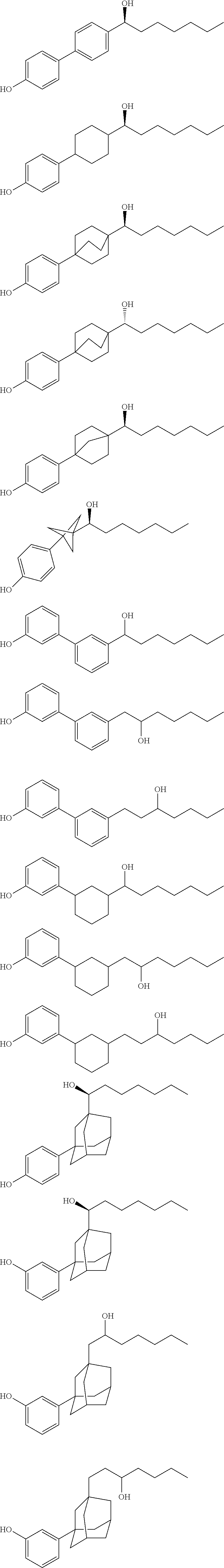

Also disclosed herein are pharmaceutically-acceptable salts and prodrugs of the carboranes and carborane analogs described herein. Pharmaceutically-acceptable salts include salts of the disclosed carboranes and carborane analogs that are prepared with acids or bases, depending on the particular substituents found on the compounds. Under conditions where the carboranes and carborane analogs disclosed herein are sufficiently basic or acidic to form stable nontoxic acid or base salts, administration of the compounds as salts can be appropriate. Examples of pharmaceutically-acceptable base addition salts include sodium, potassium, calcium, ammonium, or magnesium salt. Examples of physiologically-acceptable acid addition salts include hydrochloric, hydrobromic, nitric, phosphoric, carbonic, sulfuric, and organic acids like acetic, propionic, benzoic, succinic, fumaric, mandelic, oxalic, citric, tartaric, malonic, ascorbic, alpha-ketoglutaric, alpha-glycophosphoric, maleic, tosyl acid, methanesulfonic, and the like. Thus, disclosed herein are the hydrochloride, nitrate, phosphate, carbonate, bicarbonate, sulfate, acetate, propionate, benzoate, succinate, fumarate, mandelate, oxalate, citrate, tartarate, malonate, ascorbate, alpha-ketoglutarate, alpha-glycophosphate, maleate, tosylate, and mesylate salts. Pharmaceutically acceptable salts of a compound can be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

In some examples, the carboranes and carborane analogs disclosed herein can have an EC₅₀ of 800 nM or less at estrogen receptor beta (ERβ) (e.g., 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 9 nM or less, 8 nM or less, 7 nM or less, 6 nM or less, 5 nM or less, 4.5 nM or less, 4 nM or less, 3.5 nM or less, 3 nM or less, 2.5 nM or less, 2 nM or less, 1.5 nM or less, 1 nM or less, 0.9 nM or less, 0.8 nM or less, 0.7 nM or less, 0.6 nM or less, 0.5 nM or less, 0.4 nM or less, 0.3 nM or less, 0.2 nM or less, or 0.1 nM or less).

In some examples, the carboranes and carborane analogs disclosed herein can have an EC₅₀ of 1 pM or more at ERβ (e.g., 0.1 nM or more, 0.2 nM or more, 0.3 nM or more, 0.4 nM or more, 0.5 nM or more, 0.6 nM or more, 0.7 nM or more, 0.8 nM or more, 0.9 nM or more, 1 nM or more, 1.5 nM or more, 2 nM or more, 2.5 nM or more, 3 nM or more, 3.5 nM or more, 4 nM or more, 4.5 nM or more, 5 nM or more, 6 nM or more, 7 nM or more, 8 nM or more, 9 nM or more, 10 nM or more, 20 nM or more, 30 nM or more, 40 nM or more, 50 nM or more, 60 nM or more, 70 nM or more, 80 nM or more, 90 nM or more, 100 nM or more, 200 nM or more, 300 nM or more, 400 nM or more, 500 nM or more, 600 nM or more, or 700 nM or more).

The EC₅₀ of the carboranes and carborane analogs at ERβ can range from any of the minimum values described above to any of the maximum values described above. For example, the carboranes and carborane analogs disclosed herein can have an EC₅₀ of from 1 pM to 800 nM at ERβ (e.g., from 1 pM to 400 nM, from 400 nM to 800 nM, from 1 pM to 300 nM, from 1 pM to 200 nM, from 1 pM to 100 nM, from 1 pM to 50 nM, from 1 pM to 20 nM, from 1 pM to 10 nM, from 1 pM to 6 nM, from 1 pM to 5 nM, from 1 pM to 2 nM, from 1 pM to 1 nM, from 1 pM to 0.7 nM, from 1 pM to 0.5 nM, from 1 pM to 0.2 pM, or from 1 pM to 0.1 nM).

In some examples, the carboranes and carborane analogs disclosed herein are selective ERβ agonist. In some examples, a selective ERβ agonist is a compound that has a lower EC₅₀ at ERβ than at estrogen receptor a (ERα). The selectivity of the compounds can, in some examples, be expressed as an ERβ-to-ERα agonist ratio, which is the EC₅₀ of the compound at ERα divided by the EC₅₀ of the compound at ER. In some examples, the compounds disclosed herein can have an ERβ-to-ERα agonist ratio of 8 or more (e.g., 10 or more, 20 or more, 30 or more, 40 or more, 50 or more, 60 or more, 70 or more, 80 or more, 90 or more, 100 or more, 150 or more, 200 or more, 250 or more, 300 or more, 350 or more, 400 or more, 450 or more, 500 or more, 600 or more, 700 or more, 800 or more, 900 or more, 1000 or more, 1100 or more, 1200 or more, 1300 or more, 1400 or more, 1500 or more, 2000 or more, 2500 or more).

In some examples, the carboranes and carborane analogs can have an ERβ-to-ERα agonist ratio of 3000 or less (e.g., 2500 or less, 2000 or less, 1500 or less, 1400 or less, 1300 or less, 1200 or less, 1100 or less, 1000 or less, 900 or less, 800 or less, 700 or less, 600 or less, 500 or less, 450 or less, 400 or less, 350 or less, 300 or less, 250 or less, 200 or less, 150 or less, 100 or less, 90 or less, 80 or less, 70 or less, 60 or less, 50 or less, 40 or less, 30 or less, 20 or less, or 10 or less).

The ERβ-to-ERα agonist ratio of the carboranes and carborane analogs at ERβ can range from any of the minimum values described above to any of the maximum values described above. For example, the carboranes and carborane analogs can have an ERβ-to-ERα agonist ratio of from 8 to 3000 (e.g., from 8 to 1500, from 1500 to 3000, from 400 to 3000, from 500 to 3000, from 600 to 3000, from 700 to 3000, from 800 to 3000, from 900 to 3000, from 1000 to 3000, or from 2000 to 3000).

Methods of Making

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Katchem (Prague, Czech Republic), Aldrich Chemical Co., (Milwaukee, Wis.), Acros Organics (Morris Plains, N.J.), Fisher Scientific (Pittsburgh, Pa.), Sigma (St. Louis, Mo.), Pfizer (New York, N.Y.), GlaxoSmithKline (Raleigh, N.C.), Merck (Whitehouse Station, N.J.), Johnson & Johnson (New Brunswick, N.J.), Aventis (Bridgewater, N.J.), AstraZeneca (Wilmington, Del.), Novartis (Basel, Switzerland), Wyeth (Madison, N.J.), Bristol-Myers-Squibb (New York, N.Y.), Roche (Basel, Switzerland), Lilly (Indianapolis, Ind.), Abbott (Abbott Park, Ill.), Schering Plough (Kenilworth, N.J.), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical excipients disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high-performance liquid chromatography (HPLC) or thin layer chromatography.

Methods of Use

Also provided herein are methods of use of the compounds or compositions described herein. Also provided herein are methods for treating a disease or pathology in a subject in need thereof comprising administering to the subject a therapeutically effective amount of any of the compounds or compositions described herein.

Provided herein are methods of treating, preventing, or ameliorating fibrotic conditions in a subject using the carboranes and carborane analogs described herein. Example fibrotic conditions that can be treated or prevented using the carboranes and carborane analogs described herein (e.g., the ERβ agonists described herein) include, but are not limited to, a fibrotic condition of the lung, liver, heart, vasculature, kidney, skin, gastrointestinal tract, bone marrow, or a combination thereof. Each of these conditions is described in more detail herein.

Fibrosis of the lung (also referred to herein as “pulmonary fibrosis”) is characterized by the formation of scar tissue within the lungs, which results in a decreased function. Pulmonary fibrosis is associated with shortness of breath, which progresses to discomfort in the chest weakness and fatigue, and ultimately to loss of appetite and rapid weight-loss. Approximately 500,000 people in the U.S. and 5 million worldwide suffer from pulmonary fibrosis, and 40,000 people in the U.S. die annually from the disease. Pulmonary fibrosis has a number of causes, including radiation therapy, but can also be due to smoking or hereditary factors (Meltzer, E B et al. (2008) Orphanet J. Rare Dis. 3:8).

Pulmonary fibrosis can occur as a secondary effect in disease processes such as asbestosis and silicosis, and is known to be more prevalent in certain occupations such as coal miner, ship workers and sand blasters where exposure to environmental pollutants is an occupational hazard (Green, F H et al. (2007) Toxicol Pathol. 35:136-47). Other factors that contribute to pulmonary fibrosis include cigarette smoking, and autoimmune connective tissue disorders, like rheumatoid arthritis, scleroderma and systemic lupus erythematosus (SLE) (Leslie, K O et al. (2007) Semin Respir Crit. Care Med 28:369-78; Swigris, J J et al. (2008) Chest. 133:271-80; and Antoniou, K M et al. (2008) Curr Opin Rheumatol. 20:686-91). Other connective tissue disorders such as sarcoidosis can include pulmonary fibrosis as part of the disease (Paramothayan, S et al. (2008) Respir Med 102:1-9), and infectious diseases of the lung can cause fibrosis as a long-term consequence of infection, particularly chronic infections. Pulmonary fibrosis can also be a side effect of certain medical treatments, particularly radiation therapy to the chest and certain medicines like bleomycin, methotrexate, amiodarone, busulfan, and nitrofurantoin (Catane, R et al. (1979) Int J Radiat Oncol Biol Phys. 5:1513-8; Zisman, D A et al. (2001) Sarcoidosis Vasc Difuse Lung Dis. 18:243-52; Rakita, L et al. (1983) Am Heart J. 106:906-16; Twohig, K J et al. (1990) Clin Chest Med 11:31-54; and Witten C M. (1989) Arch PhysMed Rehabil. 70:55-7). In other embodiments, idiopathic pulmonary fibrosis can occur where no clear causal agent or disease can be identified. Increasingly, it appears that genetic factors can play a significant role in these cases of pulmonary fibrosis (Steele, M P et al. (2007) Respiration 74:601-8; Brass, D M et al. (2007) Proc Am Thorac Soc. 4:92-100 and du Bois R M. (2006) Semin Respir Crit. Care Med 27:581-8).

In some examples, the fibrotic condition of the lung can be chosen from one or more of: pulmonary fibrosis, idiopathic pulmonary fibrosis (IPF), usual interstitial pneumonitis (UIP), interstitial lung disease, cryptogenic fibrosing alveolitis (CFA), or bronchiectasis.

In other examples, the pulmonary fibrosis can include, but is not limited to, pulmonary fibrosis associated with chronic obstructive pulmonary disease (COPD), scleroderma, pleural fibrosis, chronic asthma, acute lung syndrome, amyloidosis, bronchopulmonary dysplasia, Caplans disease, Dresslers syndrome, histiocytosis X, idiopathic pulmonary haemosiderosis, lymphangiomyomatosis, mitral valve stenosis, polymyositis, pulmonary edema, pulmonary hypertension (e.g., idiopathic pulmonary hypertension (IPH)), pneumoconiosis, radiotherapy (e.g., radiation induced fibrosis), rheumatoid disease, Shavers disease, systemic lupus erythematosus, systemic sclerosis, tropical pulmonary eosinophilia, tuberous sclerosis, Weber-Christian disease, Wegeners granulomatosis, Whipples disease, or exposure to toxins or irritants (e.g., pharmaceutical drugs such as amiodarone, bleomycin, busulphan, carmustine, chloramphenicol, hexamethonium, methotrexate, methysergide, mitomycin C, nitrofurantoin, penicillamine, peplomycin, and practolol; inhalation of talc or dust, e.g., coal dust, silica). In certain embodiments, the pulmonary fibrosis is associated with an inflammatory disorder of the lung, e.g., asthma, COPD.

In some embodiments, the fibrotic condition can be a fibrotic condition of the liver (also referred to herein as “hepatic fibrosis”), such as fatty liver disease e.g., steatosis such as nonalcoholic steatohepatitis (NASH), biliary fibrosis, cholestatic liver disease (e.g., primary biliary cirrhosis (PBC), and cholangiopathies (e.g., chronic cholangiopathies)).

In certain embodiments, the fibrotic of the liver or hepatic fibrosis can be chosen from one or more of: fatty liver disease, steatosis (e.g., nonalcoholic steatohepatitis (NASH), cholestatic liver disease, primary biliary cirrhosis (PBC), biliary fibrosis, cirrhosis, alcohol induced liver fibrosis, biliary duct injury, infection or viral induced liver fibrosis, congenital hepatic fibrosis, autoimmune hepatitis, or cholangiopathies (e.g., chronic cholangiopathies).

In certain embodiments, hepatic or liver fibrosis includes, but is not limited to, hepatic fibrosis associated with alcoholism, viral infection, e.g., hepatitis (e.g., hepatitis C, B or D), autoimmune hepatitis, non-alcoholic fatty liver disease (NAFLD), progressive massive fibrosis, exposure to toxins or irritants (e.g., alcohol, pharmaceutical drugs and environmental toxins such as arsenic), alpha-1 antitrypsin deficiency, hemochromatosis, Wilsons disease, galactosemia, or glycogen storage disease. In certain embodiments, the hepatic fibrosis is associated with an inflammatory disorder of the liver.

In some embodiments, the fibrotic condition can be a fibrotic condition of the heart or vasculature, such as myocardial fibrosis. Fibrotic conditions of the heart or vasculature can include, but are not limited to, myocardial fibrosis (e.g., myocardial fibrosis associated with radiation myocarditis, a surgical procedure complication (e.g., myocardial post-operative fibrosis), vascular restenosis, atherosclerosis, cerebral disease, peripheral vascular disease, infectious diseases (e.g., Chagas disease, bacterial, trichinosis or fungal myocarditis)); granulomatous, metabolic storage disorders (e.g., cardiomyopathy, hemochromatosis); developmental disorders (e.g., endocardial fibroelastosis); arteriosclerotic, or exposure to toxins or irritants (e.g., drug induced cardiomyopathy, drug induced cardiotoxicity, alcoholic cardiomyopathy, cobalt poisoning or exposure). In certain embodiments, the myocardial fibrosis is associated with an inflammatory disorder of cardiac tissue (e.g., myocardial sarcoidosis).

In some embodiments, the fibrotic condition can be a fibrotic condition of the kidney, such as renal fibrosis (e.g., chronic kidney fibrosis). Renal fibrosis can include, but is not limited to, nephropathies associated with injury/fibrosis (e.g., chronic nephropathies associated with diabetes (e.g., diabetic nephropathy)), lupus, scleroderma of the kidney, glomerular nephritis, focal segmental glomerular sclerosis, IgA nephropathyrenal fibrosis associated with human chronic kidney disease (CKD), chronic kidney fibrosis, nephrogenic systemic fibrosis, chronic progressive nephropathy (CPN), tubulointerstitial fibrosis, ureteral obstruction (e.g., fetal partial urethral obstruction), chronic uremia, chronic interstitial nephritis, radiation nephropathy, glomerulosclerosis (e.g., focal segmental glomerulosclerosis (FSGS)), progressive glomerulonephrosis (PGN), endothelial/thrombotic microangiopathy injury, scleroderma of the kidney, HIV-associated nephropathy (HIVVAN), or exposure to toxins, irritants, chemotherapeutic agents. In one embodiment, the kidney fibrosis is mediated by a bone morphogeneic protein (BMP). In certain embodiments, the renal fibrosis is a result of an inflammatory disorder of the kidney.

In some embodiments, the fibrotic condition can be a fibrotic condition of the bone marrow. In certain embodiments, the fibrotic condition of the bone marrow is myelofibrosis (e.g., primary myelofibrosis (PMF)), myeloid metaplasia, chronic idiopathic myelofibrosis, or primary myelofibrosis. In other embodiments, bone marrow fibrosis is associated with a hematologic disorder chosen from one or more of hairy cell leukemia, lymphoma, or multiple myeloma.

In other embodiments, the bone marrow fibrosis can be associated with one or more myeloproliferative neoplasms (MPN) chosen from: essential thrombocythemia (ET), polycythemia vera (PV), mastocytosis, chronic eosinophilic leukemia, chronic neutrophilic leukemia, or other MPN.

In some examples, the fibrotic condition can be primary myelofibrosis. Primary myelofibrosis (PMF) (also referred to in the literature as idiopathic myeloid metaplasia, and Agnogenic myeloid metaplasia) is a clonal disorder of multipotent hematopoietic progenitor cells (reviewed in Abdel-Wahab, O. et al. (2009) Annu. Rev. Med. 60:233-45; Varicchio, L. et al. (2009) Expert Rev. Hematol. 2(3):315-334; Agrawal, M. et al. (2010) Cancer 1-15). The disease is characterized by anemia, splenomegaly and extramedullary hematopoiesis, and is marked by progressive marrow fibrosis and atypical megakaryocytic hyperplasia. CD34+ stem/progenitor cells abnormally traffic in the peripheral blood and multi organ extramedullary erythropoiesis is a hallmark of the disease, especially in the spleen and liver. The bone marrow structure is altered due to progressive fibrosis, neoangiogenesis, and increased bone deposits. A significant percentage of patients with PMF have gain-of-function mutations in genes that regulate hematopoiesis, including Janus kinase 2 (JAK2) (^{˜}50%) (e.g., JAK2^{V617F}) or the thrombopoietin receptor (MPL) (5-10%), resulting in abnormal megakaryocyte growth and differentiation. Studies have suggested that the clonal hematopoietic disorder leads to secondary proliferation of fibroblasts and excessive collagen deposition. Decreased bone marrow fibrosis can improve clinical signs and symptoms, including anemia, abnormal leukocyte counts, and splenomegaly.

Bone marrow fibrosis can be observed in several other hematologic disorders including, but not limited to hairy cell leukemia, lymphoma, and multiple myeloma. However, each of these conditions is characterized by a constellation of clinical, pathologic, and molecular findings not characteristic of PMF (see Abdel-Wahab, O. et al. (2009) supra at page 235).

In other embodiments, the bone marrow fibrosis can be secondary to non-hematologic disorders, including but not limited to, solid tumor metastases to bone marrow, autoimmune disorders (systemic lupus erythematosus, scleroderma, mixed connective tissue disorder, polymyositis), and secondary hyperparathyroidism associated with vitamin D deficiency (see Abdel-Wahab, O. et al. (2009) supra at page 235). In most cases, it is possible to distinguish between these disorders and PMF, although in rare cases the presence of the JAK2V617F or MPLW515L/K mutation can be used to demonstrate the presence of a clonal MPN and to exclude the possibility of reactive fibrosis.

Optionally, monitoring a clinical improvement in a subject with bone marrow fibrosis can be evaluated by one or more of: monitoring peripheral blood counts (e.g., red blood cells, white blood cells, platelets), wherein an increase in peripheral blood counts is indicative of an improved outcome. In other embodiments, clinical improvement in a subject with bone marrow fibrosis can be evaluated by monitoring one or more of: spleen size, liver size, and size of extramedullary hematopoiesis, wherein a decrease in one or more of these parameters is indicative of an improved outcome.

In other embodiments, the fibrotic condition can be a fibrotic condition of the skin. In certain embodiments, the fibrotic condition is chosen from one or more of: skin fibrosis and/or scarring, post-surgical adhesions, scleroderma (e.g., systemic scleroderma), or skin lesions such as keloids.

In certain embodiments, the fibrotic condition can be a fibrotic condition of the gastrointestinal tract. Such fibrotic conditions can be associated with an inflammatory disorder of the gastrointestinal tract, e.g., fibrosis associated with scleroderma; radiation induced gut fibrosis; fibrosis associated with a foregut inflammatory disorder such as Barretts esophagus and chronic gastritis, and/or fibrosis associated with a hindgut inflammatory disorder, such as inflammatory bowel disease (IBD), ulcerative colitis and Crohns disease. In certain embodiments, the fibrotic condition can be diffuse scleroderma.

Fibrotic conditions can further include diseases that have as a manifestation fibrotic disease of the penis, including Peyronies disease (fibrosis of the caverno us sheaths leading to contracture of the investing fascia of the corpora, resulting in a deviated and painful erection).

In some cases, the fibrotic condition can comprise Dupuytren's contracture (palmar fibromatosis).

In some cases, the fibrotic condition can comprise fibrosis associated with rheumatoid arthritis.

In certain embodiments, the fibrotic condition can be selected from pulmonary fibrosis, bronchiectasis, interstitial lung disease; fatty liver disease; cholestatic liver disease, biliary fibrosis, hepatic fibrosis; myocardial fibrosis; and renal fibrosis.

In certain embodiments, the fibrotic condition can be selected from biliary fibrosis, hepatic fibrosis, pulmonary fibrosis, myocardial fibrosis and renal fibrosis

In certain embodiments, the fibrotic condition can be selected from nonalcoholic fatty liver disease (NAFLD) and nonalcoholic steatohepatitis (NASH).

Other fibrotic conditions that can be treated with the methods and compositions of the invention include cystic fibrosis, endomyocardial fibrosis, mediastinal fibrosis, sarcoidosis, scleroderma, spinal cord injury/fibrosis.

A number of models in which fibrosis is induced are available in the art. Administration of carboranes and carborane analogs can be readily used to evaluate whether fibrosis is ameliorated in such models. Examples of such models, include but are not limited to, the unilateral ureteral obstruction model of renal fibrosis (see Chevalier et al., “Ureteral Obstruction as a Model of Renal Interstitial Fibrosis and Obstructive Nephropathy” Kidney International (2009) 75:1145-1152), the bleomycin induced model of pulmonary fibrosis (see Moore and Hogaboam “Murine Models of Pulmonary Fibrosis” Am. J. Physiol. Lung. Cell. Mol. Physiol. (2008) 294:L152-L160), a variety of liver/biliary fibrosis models (see Chuang et al., “Animal Models of Primary Biliary Cirrhosis” Clin Liver Dis (2008) 12:333-347; Omenetti, A. et al. (2007) Laboratory Investigation 87:499-514 (biliary duct-ligated model); or a number of myelofibrosis mouse models as described in Varicchio, L. (2009) supra. Regardless of the model, carboranes and carborane analogs can be evaluated in essentially three paradigms: 1) test whether carboranes and carborane analogs can inhibit the fibrotic state; 2) test whether carboranes and carborane analogs can stop fibrotic progression once initiated; and/or 3) test whether carboranes and carborane analogs can reverse the fibrotic state once initiated.

In certain embodiments, the fibrotic condition is provided in a tissue (e.g., biliary tissue, liver tissue, lung tissue, heart tissue, kidney tissue, skin tissue, gut tissue, or neural tissue). In certain embodiments, the tissue is biliary tissue. In certain embodiments, the tissue is liver tissue. In certain embodiments the tissue is lung tissue. In certain embodiments, the tissue is heart tissue. In certain embodiments, the tissue is kidney tissue. In certain embodiments, the tissue is skin tissue. In certain embodiments, the tissue is gut tissue. In certain embodiments, the tissue is bone marrow tissue. In certain embodiments, the tissue is epithelial tissue. In certain embodiments, the tissue is neural tissue.

Also provided are compositions for use, and use of, the carboranes and carborane analogs described herein, alone or in combination with another agent, for preparation of one or more medicaments for use in reducing fibrosis, or treatment of a fibrotic condition.

The examples examine the in vivo efficacy of compound 25 for the treatment of NASH.

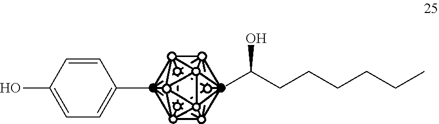

Non-Alcoholic Steatohepatitis (NASH) is increasingly recognized as the most prevalent chronic liver disease in the world and an important precedent condition to hepatocellular carcinoma (J. Gastroenterol. (2018) 53:362-376). With effective hepatitis B and C treatment and vaccination programs, respectively, largely in place, NASH mediated HCC is expected to soon overtake all other known causes of HCC (Cell. Metab. 2019 Jan. 8; 29(1):18-26). NASH prevalence is thought to approach 40% of obese adults, driving up overall incidence in lock step with a growing obesity epidemic, and represents one of the largest unmet medical needs in medicine. To date there exists no effective, FDA approved, therapy to address the pathological processes of liver steatosis, subsequent inflammation and resulting liver fibrosis associated with NASH progression. However, anti-NASH therapies remain an intense focus of the pharmaceutical industry (J Gastroenterol (2018) 53:362-376).

Like other liver pathologies, fatty liver disease displays marked sexual dimorphism such that rates of disease are higher in men than women, even when controlled for known risk factors (Adv Ther. 2017 June; 34(6):1291-1326). This dimorphism suggests an important role for sex hormone signaling such that male hormones could be reasonably hypothesized to support NASH development, and conversely, female hormones expected to play a protective role. Several lines of evidence suggest that exogenous estrogen administration can mitigate fat accumulation and adverse metabolic changes associated with high fat diet (FASEB J. 2017 January; 31(1):266-281; Mol Cell Endocrinol. 2019 Jan. 5; 479:147-158), ameliorate liver steatosis associated with a high-fat diet (Exp Biol Med (Maywood). 2017 March; 242(6):606-616, Mol Med Rep. 2016 July; 14(1):425-31), and prevent fibrosis associated with both high-fat diet (Exp Biol Med (Maywood). 2017 March; 242(6):606-616) or other liver injury (World J Gastroenterol. 2002 October; 8(5):883-7; J Gastroenterol Hepatol. 2018 March; 33(3):747-755). Together, these data highlight multiple potential beneficial mechanisms of action for therapeutic estrogen administration in NASH. However, administration of a pure, potent estrogen is not without limitations.

Therapeutic administration of steroidal endogenous estrogen preparations is associated with a number of limitations including but not limited to; exceedingly poor drug like properties, metabolic interconversion to other unwanted hormones, and unwanted severe estrogenic side-effects. For example, administration of a potent exogenous estrogen is accompanied with the fear of stimulating nascent breast cancer in a postmenopausal female NASH patient as was, with acknowledged controversy, shown to be a problem by the women's health initiative (J Steroid Biochem Mol Biol. 2014 July; 142:4-11). Likewise, in male patients, exogenous estrogen administration is associated with severe risk of deep-vein thrombosis, as was shown when DES was widely given as a prostate cancer therapeutic (Urology. 2001 August; 58(2 Suppl 1):108-13).

The earliest descriptions selective estrogen receptor modulators (SERMS) revealed that desirable estrogen pharmacology could be separated from undesirable estrogen pharmacology (Curr Clin Pharmacol. 2013 May; 8(2):135-55). Estrogen pharmacology was further advanced with the characterization of an additional, highly related, ERβ isoform that displayed differential tissue distribution and biology as compared to ERα, the originally described receptor for endogenous estrogens (Proc Natl Acad Sci USA 93:5925-5930). As ERβ biology became increasingly well characterized, it was accompanied with considerable interest in the development of therapeutic estrogens that selectively target ERβ over ERα as well as other closely related nuclear hormone receptors (Expert Opin Ther Pat. 2010 April; 20(4):507-34). One such ligand, compound 25, is a carborane based highly ERβ selective SERM.

It was hypothesized that compound 25 could provide anti-NASH efficacy through combined anti-metabolic disease, antisteatotic, and anti-fibrotic effects. To test this hypothesis compound 25 was administered once daily as two dose levels by oral gavage to male STAM model mice (Cell Metab. 2019 Jan. 8; 29(1):18-26, slide #2). STAM mice are given pharmacologic beta-cell dysfunction to mimic Type 1 Diabetes and then given a 67% fat diet to recapitulate NASH progression. Mice treated during the steatosis phase for 7 weeks tolerated both dose levels very well. Both 10 and 100 mpk dose levels of compound 25 were associated with prevention of plasma ALT and liver triglyceride levels associated with disease progression suggesting compound 25 can prevent over hepatocyte necrosis and accumulation of hepatic lipids. Notably, this efficacy is on par with an FGF21 mimic currently under development by Bristol Meyer Squibb (BMS). Critically, 100 mpk compound 25 administration was also associated with significant reduction in liver fibrosis as measured by collagen staining (Sirius Red). The magnitude of this anti-fibrotic effects was similar to those reported in the same model for an FXR agonist in clinical development by Novartis (LJN452) and the BMS FGF21 mimic.

As this is the first demonstration of an ERβ ligands efficacy in the STAM model, these findings offer considerable promise for the combination of compound 25 (or other carborane-based or carborane analog SERMS) with other anti-NASH approaches including but not limited to: SGLT inhibitors, PPARα/β/δ agonists, ACC inhibitors, FXR ligands, FGF-19 and FGF-21 or mimics, GLP-1R agonists, LOXL-2 inhibitors, Galectin-3 inhibitors, HSP-47 inhibitors, ASK-1 inhibitors, VAP-1 inhibitors, SCD inhibitors, CCR2/5 antagonists and caspase inhibitors (J Gastroenterol (2018) 53:362-376).

Likewise, as this was the first demonstration of carborane-based SERMs' anti-fibrotic effects these findings suggest that compound 25 (or other carborane-based or carborane analog SERMS) could be broadly useful in a number of fibrotic diseases including but not limited to; IPF, Calcineurin-induced renal fibrosis, Renal fibrosis NOS, Cardiac fibrosis associated with chronic heart failure (CHF), Fibrosis associated with Post-MI cardiac remodeling, Dupuytrens contracture, Fibrosis associated with RA, Liver fibrosis (viral, alcoholic, unknown origin), Peyronies disease, Keloid or other scarring (post-surgical, etc.).

Also provided herein are methods of treating, preventing, or ameliorating cancer in a subject. The methods include administering to a subject a therapeutically effective amount of one or more of the compounds or compositions described herein, or a pharmaceutically acceptable salt thereof. The compounds and compositions described herein or pharmaceutically acceptable salts thereof are useful for treating cancer in humans, e.g., pediatric and geriatric populations, and in animals, e.g., veterinary applications. The disclosed methods can optionally include identifying a patient who is or can be in need of treatment of a cancer. Examples of cancer types treatable by the compounds and compositions described herein include bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer. Further examples include cancer and/or tumors of the anus, bile duct, bone, bone marrow, bowel (including colon and rectum), eye, gall bladder, kidney, mouth, larynx, esophagus, stomach, testis, cervix, mesothelioma, neuroendocrine, penis, skin, spinal cord, thyroid, vagina, vulva, uterus, liver, muscle, blood cells (including lymphocytes and other immune system cells). Further examples of cancers treatable by the compounds and compositions described herein include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma. In some examples, the cancer can be selected from the group consisting of breast cancer, colorectal cancer, and prostate cancer.

The methods of treatment or prevention of cancer described herein can further include treatment with one or more additional agents (e.g., an anti-cancer agent or ionizing radiation). The one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein can be by the same or different routes. When treating with one or more additional agents, the compounds and compositions or pharmaceutically acceptable salts thereof as described herein can be combined into a pharmaceutical composition that includes the one or more additional agents.

For example, the compounds or compositions or pharmaceutically acceptable salts thereof as described herein can be combined into a pharmaceutical composition with an additional anti-cancer agent, such as 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C₂₂₅, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, and LCR. The additional anti-cancer agent can also include biopharmaceuticals such as, for example, antibodies.

Many tumors and cancers have viral genome present in the tumor or cancer cells. For example, Epstein-Barr Virus (EBV) is associated with a number of mammalian malignancies. The compounds disclosed herein can also be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells. The compounds disclosed herein can also be used in combination with viral based treatments of oncologic disease.

Also described herein are methods of suppressing tumor growth in a subject. The method includes contacting at least a portion of the tumor with a therapeutically effective amount of a compound or composition as described herein, and optionally includes the step of irradiating at least a portion of the tumor with a therapeutically effective amount of ionizing radiation. As used herein, the term ionizing radiation refers to radiation comprising particles or photons that have sufficient energy or can produce sufficient energy via nuclear interactions to produce ionization. An example of ionizing radiation is x-radiation. A therapeutically effective amount of ionizing radiation refers to a dose of ionizing radiation that produces an increase in cell damage or death when administered in combination with the compounds described herein. The ionizing radiation can be delivered according to methods as known in the art, including administering radiolabeled antibodies and radioisotopes.

Also described herein are methods of treating an inflammatory disease in a subject. The methods can include administering to the subject a therapeutically effective amount of a compound or a composition as described herein. Inflammatory diseases include, but are not limited to, acne vulgaris, ankylosing spondylitis, asthma, autoimmune diseases, Celiac disease, chronic prostatitis, Crohn's disease, glomerulonephritis, hidradenitis suppurativa, inflammatory bowel diseases, pelvic inflammatory disease, psoriasis, reperfusion injury, rheumatoid arthritis, sarcoidosis, vasculitis, interstitial cystitis, type 1 hypersensitivities, systemic sclerosis, dermatomyositis, polymyositis, and inclusion body myositis. In some examples, the inflammatory disease is selected from the group consisting of arthritis and inflammatory bowel disease.

The methods of treatment of inflammatory diseases described herein can further include treatment with one or more additional agents (e.g., an anti-inflammatory agent). The one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein can be by the same or different routes. When treating with one or more additional agents, the compounds and compositions or pharmaceutically acceptable salts thereof as described herein can be combined into a pharmaceutical composition that includes the one or more additional agents.

Also disclosed herein are methods of treating a neurodegenerative disease in a subject. The methods can comprise administering to the subject a therapeutically effective amount of a compound or a composition as described herein. Neurodegenerative diseases include, but are not limited to, Alzheimer's disease, amyotrophic lateral sclerosis (ALS), Alpers' disease, batten disease, Benson's syndrome, Cerebro-oculo-facio-skeletal (COFS) syndrome, corticobasal degeneration, Creutzfeldt-Jakob disease, dementias, Friedreich's ataxia, Gerstmann-Strussler-Scheinker disease, Huntington's disease, Lewy body syndrome, Leigh's disease, monomelic amyotrophy, motor neuron diseases, multiple system atrophy, opsoclonus myoclonus, progressive multifocal leukoencephalopathy, Parkinson's disease, Prion diseases, primary progressive aphasia, progressive supranuclear palsy, spinocerebellar ataxia, spinal muscular atrophy, kuru, and Shy-Drager syndrome.

Also disclosed herein are methods of treating a psychotropic disorder in a subject. The methods can comprise administering to the subject a therapeutically effective amount of a compound or a composition as described herein. Psychotropic disorders include, but are not limited to, attention deficit disorder (ADD), attention deficit hyperactive disorder (ADHD), anorexia nervosa, anxiety, dipolar disorder, bulimia, depression, insomnia, neuropathic pain, mania, obsessive compulsive disorder (OCD), panic disorder, premenstrual dysphoric disorder (PMDD), mood disorder, serotonin syndrome, schizophrenia, and seasonal affective disorder.

The compounds described herein can also be used to treat other ERβ-related (ERβ-mediated) diseases, including cardiovascular diseases (e.g., heart attack, heart failure, ischemic stroke, arrhythmia), benign prostatic hyperplasia, and osteoporosis.

Also disclosed herein are methods of imaging a cell or a population of cells expressing ERβ within or about a subject. The methods can comprise administering to the subject an amount of a compound or a composition as described herein; and detecting the compound or the composition. The detecting can involve methods known in the art, for example, positron emission tomography *PET), single-photon emission computed tomography (SPECT), magnetic resonance imaging (MRI), X-ray, microscopy, computed tomography (CT). In some examples, the compound or composition can further comprise a detectable label, such as a radiolabel, fluorescent label, enzymatic label, and the like. In some examples, the detectable label can comprise a radiolabel, such as ¹⁰B. Such imaging methods can be used, for example, for assessing the extent of a disease and/or the target of a therapeutic agent.

The methods and compounds as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of the disease or disorder), during early onset (e.g., upon initial signs and symptoms of the disease or disorder), or after an established development of the disease or disorder. Prophylactic administration can occur for several days to years prior to the manifestation of symptoms of a disease or disorder. Therapeutic treatment involves administering to a subject a therapeutically effective amount of the compounds and compositions or pharmaceutically acceptable salts thereof as described herein after the disease or disorder is diagnosed.

Compositions, Formulations and Methods of Administration

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that a therapeutically effective amount of the compound is combined with a suitable excipient in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the excipients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib.

In certain examples, compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; diluents such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are pharmaceutical compositions that comprise a compound disclosed herein in combination with a pharmaceutically acceptable excipient. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

Also disclosed are kits that comprise a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

A number of embodiments of the invention have been described. Nevertheless, it will be understood that various modifications may be made without departing from the spirit and scope of the invention. Accordingly, other embodiments are within the scope of the following claims.

### EXAMPLES

The following examples are set forth to illustrate the methods and results according to the disclosed subject matter. These examples are not intended to be inclusive of all aspects of the subject matter disclosed herein, but rather to illustrate representative methods and results. These examples are not intended to exclude equivalents and variations which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process. Only reasonable and routine experimentation will be required to optimize such process conditions.

¹H- and ¹³C-NMR spectra were recorded at The Ohio State University College of Pharmacy using a Bruker AVIII400HD NMR spectrometer or a Bruker DRX400 NMR spectrometer, or at The Ohio State University Campus Chemical Instrumentation Center using a Bruker Ascend 700 MHz NMR at. Chemical shifts (6) are reported in ppm from internal deuterated chloroform or deuterated acetone. Coupling constants are reported in Hz. ¹³C NMR spectra are fully decoupled. NMR spectra were analyzed with Mnova Lite SE (Mestrelab Research, Bajo, Spain). Melting points were obtained on a Thomas Hoover “UNI-MELT” capillary melting apparatus. Optical rotation was measured on a JASCO J-810 spectropolarimeter. Accurate and high resolution mass spectra were obtained from Ohio State University Campus Chemical Instrumentation Center using a Waters Micromass LCT mass spectrometer or a Waters Micromass Q-TOF II mass spectrometer, from The Ohio State University College of Pharmacy using a Waters Micromass Q-TOF micro mass spectrometer or a Thermo LTQ Orbitrap mass spectrometer, or from the University of Illinois Urbana-Champaign Mass Spectrometry Laboratory using a Waters Micromass 70-VSE mass spectrometer. For all carborane-containing compounds, the found mass corresponding to the most intense peak of the theoretical isotopic pattern was reported. Measured patterns agreed with calculated patterns.

Silica gel 60 (0.063-0.200 mm), used for gravity column chromatography. Reagent-grade solvents were used for silica gel column chromatography. Precoated glass-backed TLC plates with silica gel 60 F254 (0.25-mm layer thickness) from Dynamic Adsorbents (Norcross, Ga.) were used for TLC. General compound visualization for TLC was achieved by UV light. Carborane-containing compounds were selectively visualized by spraying the plate with a 0.06% PdCl₂/1% HCl solution and heating at 120° C., which caused the slow (15-45 s) formation of a gray spot due to the reduction of Pd²⁺ to Pd⁰. Chiral analytical HPLC was conducted using a CHIRAL PAK® IB-3 column (250×4.6 mm, 3 μm particle size) supplied by Chiral Technologies, PA, USA using on a Hitachi HPLC system (L-2130) with a Windows based data acquisition and Hitachi Diode array detector (L-2455). HPLC-grade solvents were used for HPLC.

Anhydrous solvents for reactions were purchased directly from Acros Organics (Morris Plains, N.J.) or from Sigma Aldrich (Milwaukee, Wis.). Other solvents and chemicals were obtained from standard vendors. Unless specified otherwise, all reactions were carried out under argon atmosphere.

### Example 1

To a solution of 1-(4-methoxyphenyl)-1,12-dicarba-closo-dodecaborane (Endo Y et al. Chemistry & Biology, 2001, 8, 341-355) (500 mg, 2 mmol) in anhydrous dimethoxyethane (DME, 40 mL) was added n-butyllithium (1 mL, 2.5 mmol, 2.5 M solution in hexanes) at 0° C. The reaction mixture was stirred at room temperature for 1.5 h. A quantity of 0.49 mL (3.0 mmol) 1-iodoheptane was added at 0° C. Following stirring at room temperature for 4 h, the reaction mixture was carefully poured into 60 mL of 1 M HCl and extracted with ethyl acetate. The organic phase was washed with a 10% sodium thiosulfate solution and brine and dried over MgSO₄. The solvents were evaporated, and the residue purified by silica gel column chromatography (hexanes, R_{f}. 0.38) to yield 550 mg (79%) product as a white solid which had a melting point of 45-46° C.

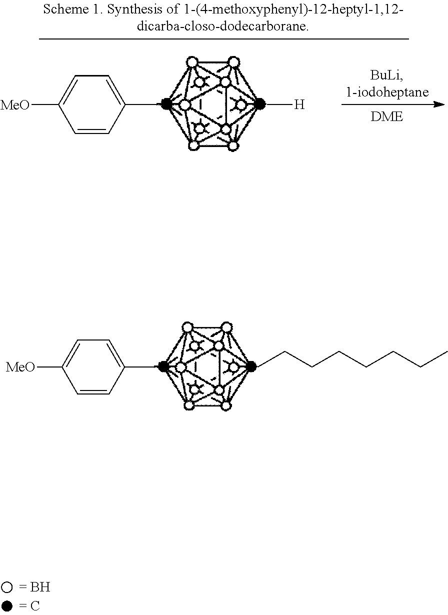

¹H NMR (CDCl₃): δ 0.87 (t, 3H, CH₃), 1.08-1.28 (m, 10H, 5×CH₂), 1.64 (m, 2H, C_{carborane}—CH₂), 1.85-3.0 (br. m, 10H, BH), 3.74 (s, 3H, OCH₃), 6.67 (d, 2H, arom., J=9.0 Hz), 7.11 (d, 2H, arom., J=9.0 Hz). ¹³C NMR (CDCl₃): δ 14.21, 22.73, 29.02, 29.24, 29.67, 31.82, 38.05, 55.39, 80.92, 113.36, 128.49, 128.97, 159.61. Accurate mass HRMS (EI+): m/z calcd. For C₁₆H₃₂B₁₀O (M)⁺ 348.3465, found 348.3461.

### Example 2

To a solution of 1-(4-methoxyphenyl)-1,12-dicarba-closo-dodecaborane (Endo Y et al. Chemistry & Biology, 2001, 8, 341-355) (500 mg, 10 mmol) in anhydrous dimethoxyethane DME (100 mL) was added n-butyllithium (4.8 mL, 12 mmol, 2.5 M solution in hexanes) at 0° C. The reaction mixture was stirred at room temperature for 1.5 h. A quantity of 1.83 mL (13 mmol) 1-heptanal was added at 0° C. Following stirring at room temperature overnight, the reaction mixture was carefully poured into 150 mL of 1 M HCl and extracted with ethyl acetate. The organic phase was washed with brine and dried over MgSO₄. The solvents were evaporated and the residue purified by column chromatography (hexanes/EtOAc, 19/1, v/v, R_{f}. 0.43) to yield 3.0 g (82%) of a white solid which had a melting point of 104-105° C.

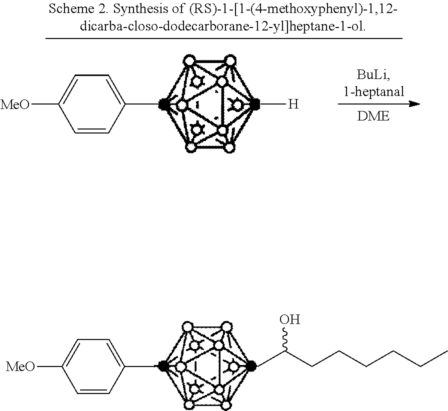

¹H NMR (CDCl₃): δ 0.88 (t, 3H, CH₃), 1.15-1.30 (m, 8H, 4×CH₂), 1.38-1.47 (m, 2H, CH₂), 1.59 (br. s, 1H, OH), 1.85-3.0 (br. m, 10H, BH), 3.47 (m, 1H, CH), 3.74 (s, 3H, OCH₃), 6.68 (d, 2H, arom., J=9.0 Hz), 7.12 (d, 2H, arom., J=9.0 Hz). ¹³C NMR (CDCl₃): δ 14.20, 22.71, 26.59, 28.98, 31.83, 36.92, 55.39, 73.10, 83.53, 86.36, 113.41, 128.43, 128.84, 159.73. Accurate mass HRMS (EI+): m/z calcd for C₁₆H₃₂B₁₀O₂ (M)⁺ 364.3414, found 364.3423.

### Example 3

For the synthesis (RS)-1-[1-(4-methoxyphenyl)-1,12-dicarba-closo-dodecaborane-12-yl]butane-1-ol, the procedure and conditions described for the synthesis of (RS)-1-[1-(4-methoxyphenyl)-1,12-dicarba-closo-dodecaborane-12-yl]heptane-1-ol were adapted using 500 mg (2 mmol) 1-(4-methoxyphenyl)-1,12-dicarba-closo-dodecaborane (Endo Y et al. Chemistry & Biology, 2001, 8, 341-355) as the starting material.

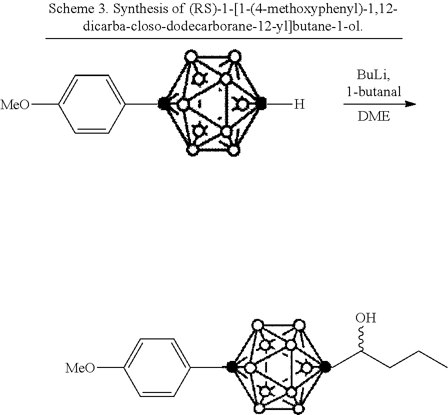

Yield: 500 mg (78%, white solid), R_{f}. 0.33 (hexanes/EtOAc, 19/1, v/v), m.p.: 96-97° C. ¹H NMR (CDCl₃): δ 0.87 (t, 3H, CH₃), 1.16-1.27 (m, 4H, 2×CH₂), 1.35-1.39 (m, 2H, CH₂), 1.45-152 (m, 2H, CH₂), 1.59 (br. s, 1H, OH), 1.85-3.0 (br. m, 10H, BH), 3.49 (m, 1H, CH), 3.74 (s, 3H, OCH₃), 6.68 (d, 2H, arom., J=9.0 Hz), 7.12 (d, 2H, arom., J=9.0 Hz). ¹³C NMR (CDCl₃): δ 13.75, 19.82, 38.94, 55.40, 72.84, 83.54, 86.34, 113.42, 128.43, 128.84, 159.73. Accurate mass HRMS (EI+): m/z calcd for C₁₃H₂₆B₁₀O₂ (M)⁺ 322.2943, found 322.2929.

### Example 4

For the synthesis of (RS)-1-[1-(4-methoxyphenyl)-1,12-dicarba-closo-dodecaborane-12-yl]-6-methylheptane-1-ol, the procedure and conditions described for the synthesis of (RS)-1-[1-(4-methoxyphenyl)-1,12-dicarba-closo-dodecaborane-12-yl]heptane-1-ol were adapted using 1 g (4 mmol) 1-(4-methoxyphenyl)-1,12-dicarba-closo-dodecaborane (Endo Y et al. Chemistry & Biology, 2001, 8, 341-355) and 0.75 g (5.85 mmol) of 6-methylheptanal (Kuhnke J & Bohlman F., Tetrahedron Lett. 1985, 26, 3955-3958) as the starting materials.

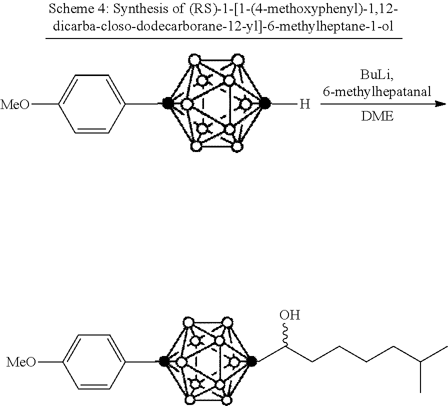

Yield: 1.16 mg (77%, white solid), R_{f}. 0.49 (hexanes/EtOAc, 19/1, v/v), m.p.: 95-96° C. ¹H NMR (CDCl₃): δ 0.85 (s, 3H, CH₃), 0.86 (s, 3H, CH₃), 1.11-1.28 (m, 6H, 3×CH₂), 1.39-1.44 (m, 2H, CH₂), 1.47-1.53 (1m, 1H, CH), 1.45-152 (m, 2H, CH₂), 1.58 (br. s, 1H, OH), 1.85-3.0 (br. m, 10H, BH), 3.47 (m, 1H, CH), 3.74 (s, 3H, OCH₃), 6.68 (d, 2H, arom., J=9.0 Hz), 7.12 (d, 2H, arom., J=9.0 Hz). ¹³C NMR (CDCl₃): δ 22.71, 22.78, 26.89, 27.08, 28.04, 36.94, 38.95, 55.40, 73.10, 83.54, 86.39, 113.42, 128.43, 128.84, 159.73. Accurate mass HRMS (EI+): m/z calcd for C₁₇H₃₄B₁₀O₂ (M)⁺ 378.3571, found 378.3576.

### Example 5

For the synthesis of (RS)-1-[1-(4-methoxyphenyl)-1,12-dicarba-closo-dodecaborane-12-yl]-3-phenylpropan-1-ol, the procedure and conditions described for the synthesis of (RS)-1-[1-(4-Methoxyphenyl)-1,12-dicarba-closo-dodecaborane-12-yl]heptane-1-ol were adapted using 250 mg (1 mmol) 1-(4-methoxyphenyl)-1,12-dicarba-closo-dodecaborane (Endo Y et al. Chemistry & Biology, 2001, 8, 341-355) and 0.17 g (1.5 mmol) of 3-phenylheptanal as the starting materials.

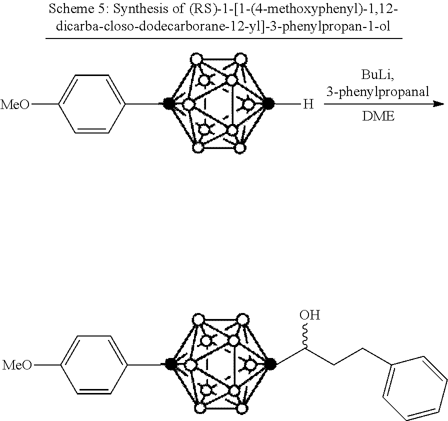

Yield: 344 mg (90%, white solid), R_{f}. 0.27 (hexanes/EtOAc, 19/1, v/v), m.p.: 123-124° C. ¹H NMR (CDCl₃): δ 01.49-1.77 (m, 2H, CH₂), 1.69 (br. s, 1H, OH), 1.85-3.0 (br. m, 10H, BH), 2.51-2.83 (m, 2H, CH₂), 3.48 (m, 1H, CH), 3.74 (s, 3H, OCH₃), 6.68 (d, 2H, arom., J=9.0 Hz), 7.11 (d, 2H, arom., J=9.0 Hz), 7.14 (d, 2H, arom.), 7.20 (t, 1H, arom.), 7.28 (t, 2H, arom.). ¹³C NMR (CDCl₃): δ 32.69, 38.29, 55.39, 72.31, 83.64, 86.02, 113.42, 126.19, 128.41, 128.52, 128.61, 128.77, 141.15, 159.74. Accurate mass HRMS (EI+): m/z calcd for C₁₈H₂SB₁₀O₂ (M)⁺ 384.3102, found 38.3101.

### Example 6

For the synthesis of (RS)-(2,3-dihydro-1H-inden-5-yl)-[1-(4-methoxyphenyl)-1,12-dicarba-closo-dodecaborane-12-yl]methanol, the procedure and conditions described for the synthesis of (RS)-1-[1-(4-Methoxyphenyl)-1,12-dicarba-closo-dodecaborane-12-yl]heptane-1-ol were adapted using 450 mg (1.8 mmol) 1-(4-methoxyphenyl)-1,12-dicarba-closo-dodecaborane (Endo Y et al. Chemistry & Biology, 2001, 8, 341-355) and 100 g (0.69 mmol) of 5-formylindane as the starting materials. Subsequent to the reaction, excess 1-(4-methoxyphenyl)-1,12-dicarba-closo-dodecaborane was initially recovered by column chromatography using hexanes only.

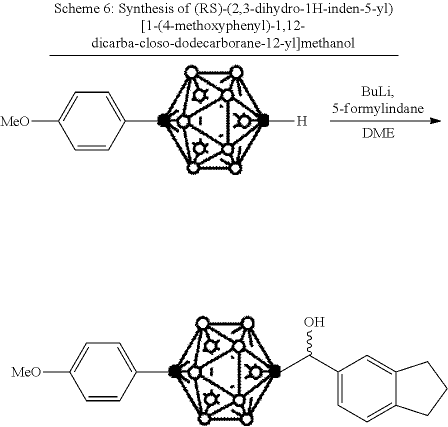

Yield: 240 mg (79%, white solid), R_{f}. 0.28 (hexanes/EtOAc, 19/1, v/v), m.p.: 123-124° C. ¹H NMR (CDCl₃): δ 1.85-3.0 (br. m, 10H, BH), 2.06-2.10 (m, 3H, CH₂, OH), 2.89 (m, 4H, 2×CH₂), 3.74 (s, 3H, OCH₃), 4.46 (s, 1H, CH), 6.66 (d, 2H, arom., J=9.0 Hz), 6.92 (d, 1H, arom.), 7.03 (s, 1H, arom.), 7.09 (d, 2H, arom., J=9.0 Hz), 7.15 (d, 2H, arom.). ¹³C NMR (CDCl₃): δ 25.56, 32.77, 32.95, 55.39, 76.11, 83.65, 85.84, 113.39, 122.74, 123.95, 124.92, 128.41, 128.86, 138.24, 144.29, 144.95, 159.71. Accurate mass HRMS (EI+): m/z calcd for C₁₉H₂₈B₁₀O₂ (M)⁺ 396.3102, found 396.3096.

### Example 7

Pyridinium chlorochromate (PCC, 2.0 g, 9.34 mmol) was suspended in anhydrous DCM (50 mL). A solution of (RS)-1-[1-(4-methoxyphenyl)-1,12-dicarba-closo-dodecaborane-12-yl]heptane-1-ol (1.7 g, 4.67 mmol) in anhydrous DCM (15 mL) was then added to give a dark reaction mixture, which was stirred at room temperature overnight. Diethylether (60 mL) was added and then molecular sieve followed by stirring for 1 h. The supernatant was decanted and the insoluble residue was washed with dry ether (3×20 mL). The combined organic phases were passed through a short column of florisil followed by evaporation. The residue was purified by silica gel column chromatography (hexanes, R_{f}. 0.13) to yield 1.6 g (95%) of a white wax-like solid which had a melting point of 36-37° C.

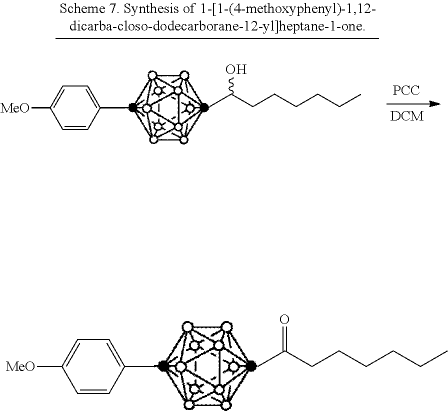

¹H NMR (CDCl₃): δ 0.87 (t, 3H, CH₃), 1.14-1.46 (m, 8H, 4×CH₂), 1.85-3.0 (br. m, 10H, BH), 2.39 (m, 2H, C(O)—CH₂), 3.74 (s, 3H, OCH₃), 6.69 (d, 2H, arom., J=9.0 Hz), 7.10 (d, 2H, arom., J=8.9 Hz). ¹³C NMR (CDCl₃): δ 14.14, 22.58, 23.60, 28.51, 31.60, 39.39, 55.41, 83.75, 85.64, 113.50, 128.28, 128.73, 159.92, 195.48. Accurate mass HRMS (EI+): m/z calcd for C₁₆H₃₀B₁₀O₂ (M)⁺ 362.3257, found 362.3254.

### Example 8

Borane-tetrahydrofuran complex (16.5 mL, 16.5 mmol, 1.0 M solution in THF, stabilized with 0.005 M N-isopropyl-N-methyl-tert-butylamine (NIMBA)) followed by (S)-2-methyl-CBS-oxazaborolidine [(S)-MeCBS] (1.65 mL, 1.65 mmol, 1.0 M solution in toluene) were added to 15 mL anhydrous THF. The reaction mixture was stirred at room temperature for 10 minutes and 1-[1-(4-methoxyphenyl)-1,12-dicarba-closo-dodecaborane-12-yl]heptane-1-one (600 mg, 1.65 mmol) in 15 mL of anhydrous THF was added slowly over a period of 2 h at 25° C. The reaction mixture was stirred for additional 6 h at room temperature and then carefully quenched by addition of 2.0 M HCl (30 mL) in small portions to control H₂ development. Diethyl ether (50 mL) was added and the organic phase was washed brine and saturated NaHCO₃. The organic phase was dried over MgSO₄, filtered, and evaporated. The residue was purified by silica gel column chromatography (hexanes/EtOAc, 19/1, v/v) to yield a white solid. Based on chiral HPLC (CHIRALPAK IB-3 [Chiral Technologies, INC.], hexanes/DCM [9/1], 1 mL flow rate), and analysis of the ¹H NMR spectrum of the corresponding Mosher ester, the enantiomeric excess (ee) was estimated to be >85%. The absolute configuration was determined by analysis of the ¹H NMR spectrum of the corresponding Mosher ester.

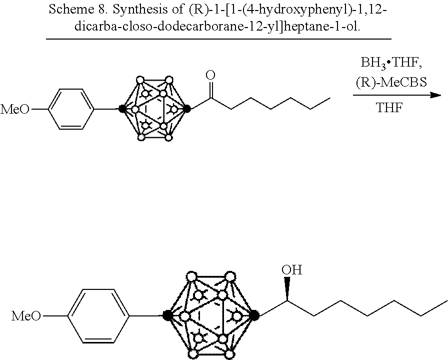

Yield: 440 mg (73%), R_{f}. 0.43 (hexanes/EtOAc, 19/1, v/v), m.p.: 95-96° C., [α]_{D}^{20° C.}=+27 (0.1, DCM). ¹H NMR (CDCl₃): δ 0.87 (t, 3H, CH₃), 1.15-1.31 (m, 8H, 4×CH₂), 1.38-1.48 (m, 2H, CH₂), 1.58 (br. s, 1H, OH), 1.85-3.0 (br. m, 10H, BH), 3.47 (m, 1H, CH), 3.74 (s, 3H, OCH₃), 6.68 (d, 2H, arom., J=9.0 Hz), 7.12 (d, 2H, arom., J=9.0 Hz). ¹³C NMR (CDCl₃): δ 14.20, 22.72, 26.60, 28.98, 31.83, 36.92, 55.40, 73.10, 83.53, 86.39, 113.42, 128.43, 128.85, 159.73. Accurate mass HRMS (EI+): m/z calcd for C₁₆H₃₂B₁₀O₂ (M)⁺ 364.3414, found 364.3417.

### Example 9

For the synthesis of (R)-1-[1-(4-methoxyphenyl)-1,12-dicarba-closo-dodecaborane-12-yl]heptane-1-ol, the procedure and conditions described for the synthesis of (s)-1-[1-(4-methoxyphenyl)-1,12-dicarba-closo-dodecaborane-12-yl]heptane-1-ol were adapted using 500 mg (1.38 mmol) of 1-[1-(4-methoxyphenyl)-1,12-dicarba-closo-dodecaborane-12-yl]heptane-1-one and 1.38 mL (1.38 mmol, 1.0 M solution in toluene) of (R)-MeCBS. The residue was purified by silica gel column chromatography (hexanes/EtOAc, 19/1, v/v) to yield a white solid. Based on chiral HPLC (CHIRALPAK IB-3 [Chiral Technologies, INC.], hexanes/DCM [9/1], 1 mL flow rate), the enantiomeric excess (ee) was estimated to be >85%. The assignment of the absolute configuration was derived from the analysis of the ¹H-NMR spectrum of the Mosher ester of (S)-1-[1-(4-methoxyphenyl)-1,12-dicarba-closo-dodecaborane-12-yl]heptane-1-ol.

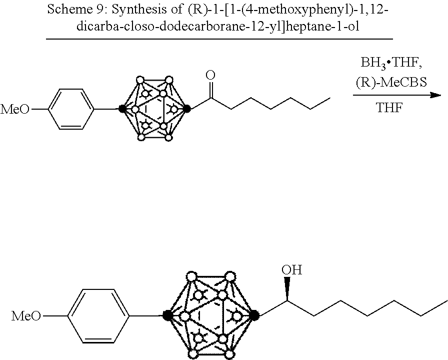

Yield: 400 mg (80%), R_{f}. 0.43 (hexanes/EtOAc, 19/1, v/v), m.p.: 95-96° C., [α]D^{20° C.}=−24° (0.1, DCM). ¹H NMR (CDCl₃): δ 0.87 (t, 3H, CH₃), 1.15-1.31 (m, 8H, 4×CH₂), 1.38-1.47 (m, 2H, CH₂), 1.57 (br. s, 1H, OH), 1.85-3.0 (br. m, 10H, BH), 3.47 (m, 1H, CH), 3.74 (s, 3H, OCH₃), 6.68 (d, 2H, arom., J=9.0 Hz), 7.12 (d, 2H, arom., J=9.0 Hz). ¹³C NMR (CDCl₃): δ 14.20, 22.72, 26.60, 28.99, 31.83, 36.92, 55.40, 73.10, 83.54, 86.39, 113.42, 128.43, 128.85, 159.73. Accurate mass HRMS (EI+): m/z calcd for C₁₆H₃₂B₁₀O₂ (M)⁺ 364.3414, found 364.3406.

### Example 10

To a solution of 1-(4-methoxyphenyl)-12-heptyl-1,12-dicarba-closo-dodecaborane (600 mg, 1.72 mmol) in anhydrous DCM (40 mL) was added boron tribromide (3.4 mL, 3.4 mmol), 1 M solution in DCM) at 0° C. The reaction mixture was stirred at room temperature overnight, poured carefully into ice-cold 1 M HCl (60 mL) and extracted with DCM. The organic phase was washed with a 10% sodium thiosulfate solution and brine and dried over MgSO₄. The solvents were evaporated and the residue purified by silica gel column chromatography (hexanes/EtOAc, 9/1, v/v) to yield a white solid. Further purification can be achieved by recrystallization from pentane or hexanes (−20° C.).

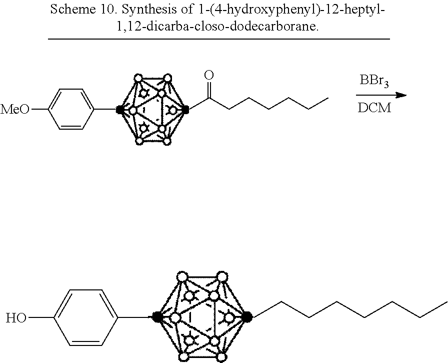

Yield: 380 mg (66%), R_{f}. 0.36 (hexanes/EtOAc, 9/1, v/v), m.p.: 114-115° C. ¹H NMR (CDCl₃): δ 0.87 (t, 3H, CH₃), 1.08-1.29 (m, 10H, 5×CH₂), 1.64 (m, 2H, C_{carborane}—CH₂), 1.85-3.0 (br. m, 10H, BH), 4.68 (br. s, 1H, OH), 6.60 (d, 2H, arom., J=8.8 Hz), 7.07 (d, 2H, arom., J=8.8 Hz). ¹³C NMR (CDCl₃): δ 14.20, 22.73, 29.02, 29.23, 29.67, 31.87, 38.04, 80.82, 80.98, 81.21, 114.83, 128.76, 129.30, 155.59. Accurate mass HRMS (ESI): m/z calcd for C₁₅H₂₉B₁₀O (M−1)⁻ 333.3216, found 333.3213.

### Example 11

To a solution of (RS)-1-[1-(4-methoxyphenyl)-1,12-dicarba-closo-dodecaborane-12-yl]heptane-1-ol (570 mg, 1.57 mmol) in anhydrous DCM (40 mL) was added boron tribromide (4.7 mL, 4.7 mmol, 1 M solution in DCM) at 0° C. The reaction mixture was stirred at room temperature overnight, poured carefully into ice-cold 1 M HCl (60 mL) and extracted with DCM. The organic phase was washed with a 10% sodium thiosulfate solution and brine and dried over MgSO₄. The solvents were evaporated and the residue purified by silica gel column chromatography (hexanes/EtOAc, 9/1, v/v) to yield a white solid. Further purification can be achieved by recrystallization from hexanes/i-propanol [24:1] and washing the obtained residue with ice-cold pentane.

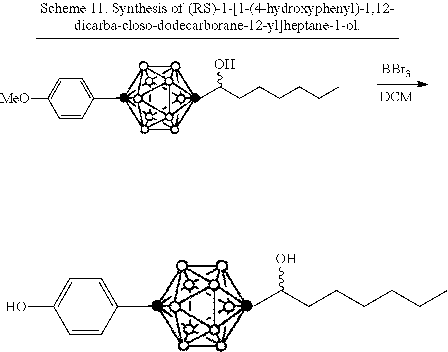

Yield: 400 mg (73%), R_{f}. 0.23 (hexanes/EtOAc, 9/1, v/v), m.p.: 129-130° C. ¹H NMR (CDCl₃): δ 0.87 (t, 3H, CH₃), 1.14-1.30 (m, 8H, 4×CH₂), 1.38-1.45 (m, 2H, CH₂), 1.62-1.63 (m, ˜2H, OH & H₂O), 1.85-3.0 (br. m, 10H, BH), 3.46 (m, 1H, CH), 4.96 (br. s, 1H, OH), 6.61 (d, 2H, arom., J=8.8 Hz), 7.07 (d, 2H, arom., J=8.9 Hz). ¹³C NMR (CDCl₃): δ 14.19, 22.71, 26.58, 28.97, 31.82, 36.91, 73.14, 83.57, 86.37, 114.90, 128.68, 129.06, 155.82. Accurate mass HRMS (ESI): m/z calcd for C₁₅H₃₁B₁₀O₂ (M+1)⁻ 351.3329, found 351.3322.

### Example 12

The procedure and conditions described for the synthesis of (RS)-1-[1-(4-hydroxyphenyl)-1,12-dicarba-closo-dodecaborane-12-yl]heptane-1-ol were adapted using 450 mg (1.4 mmol) (RS)-1-[1-(4-methoxyphenyl)-1,12-dicarba-closo-dodecaborane-12-yl]butane-1-ol as the starting material. Purification of the products is carried out by silica gel column chromatography (hexanes/EtOAc, 9/1, v/v) to yield a white solid. Further purification can be achieved by recrystallization from hexanes/i-propanol [24:1] and washing the obtained residue with ice-cold pentane.

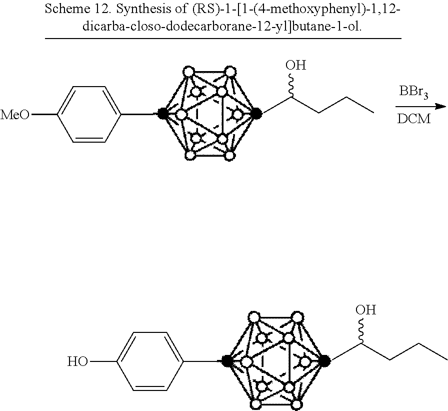

Yield: 265 mg (62%), R_{f}. 0.22 (hexanes/EtOAc, 9/1, v/v), m.p.: 184-185° C. ¹H NMR (CDCl₃): δ 0.87 (t, 3H, CH₃), 1.15-1.26 (m, 2H, CH₂), 1.33-1.51 (m, 2H, CH₂), 1.55 (br. s, ˜2H, OH & H₂O), 1.85-3.0 (br. m, 10H, BH), 3.48 (m, 1H, CH), 4.69 (br. s, ˜1H, OH), 6.61 (d, 2H, arom., J=8.8 Hz), 7.07 (d, 2H, arom., J=8.8 Hz). ¹³C NMR (CDCl₃): δ 13.75, 19.82, 38.95, 72.86, 83.41, 86.39, 114.90, 128.71, 129.15, 155.75. Accurate mass HRMS (ESI): m/z calcd for C₁₂H₂₃B₁₀O₂ (M−1)⁻ 307.2701, found 307.2700.

### Example 13

The procedure and conditions described for the synthesis of (RS)-1-[1-(4-hydroxyphenyl)-1,12-dicarba-closo-dodecaborane-12-yl]heptane-1-ol were adapted using 550 mg (1.46 mmol) (RS)-1-[1-(4-methoxyphenyl)-1,12-dicarba-closo-dodecaborane-12-yl]-6-methylheptane-1-ol as the starting material. Purification of the products is carried out by silica gel column chromatography (hexanes/EtOAc, 9/1, v/v) to yield a white solid. Further purification can be achieved by recrystallization from hexanes/i-propanol [24:1] and washing the obtained residue with ice-cold pentane.

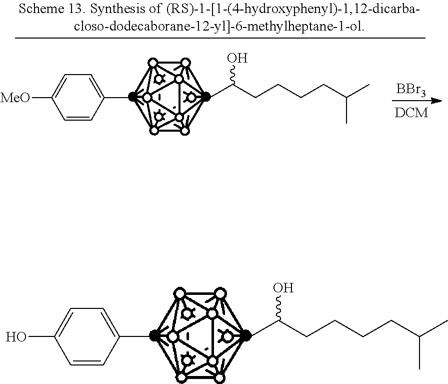

Yield: 340 mg (72%), R_{f}. 0.23 (hexanes/EtOAc, 9/1, v/v), m.p.: 120-121° C. ¹H NMR (CDCl₃): δ 0.84 (s, 3H, CH₃), S 0.85 (s, 3H, CH₃), 1.10-1.28 (m, 6H, 3×CH₂), 1.38-1.45 (m, 2H, CH₂), 1.46-1.52 (m, 1H, CH), 1.61 (br. s, ˜2H, OH & H₂O), 1.85-3.0 (br. m, 10H, BH), 3.47 (m, 1H, CH), 4.88 (br. s, ˜1H, OH), 6.61 (d, 2H, arom., J=8.8 Hz), 7.07 (d, 2H, arom., J=8.8 Hz). ¹³C NMR (CDCl₃): δ 22.71, 22.78, 26.88, 27.07, 28.04, 36.93, 38.94, 73.13, 83.33, 86.38, 114.90, 128.69, 129.09, 155.80. Accurate mass HRMS (ESI): m/z calcd for C₁₆H₃₁B₁₀O₂ (M−1)⁻ 363.3322, found 363.3331.

### Example 14

The procedure and conditions described for the synthesis of (RS)-1-[1-(4-hydroxyphenyl)-1,12-dicarba-closo-dodecaborane-12-yl]heptane-1-ol were adapted using 250 mg (0.65 mmol) (RS)-1-[1-(4-methoxyphenyl)-1,12-dicarba-closo-dodecaborane-12-yl]-3-phenylpropan-1-ol as the starting material. Purification of the products is carried out by silica gel column chromatography (hexanes/EtOAc, 9/1, v/v) to yield a white solid. Further purification can be achieved by recrystallization from hexanes/i-propanol [24:1] and washing the obtained residue with ice-cold pentane.

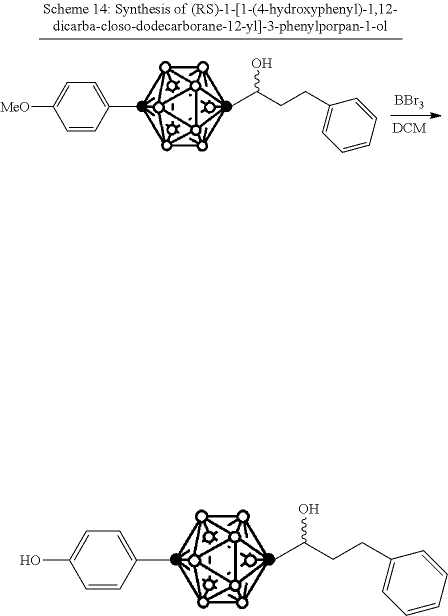

Yield: 200 mg (83%), R_{f}. 0.15 (hexanes/EtOAc, 9/1, v/v), m.p.: 135-136° C. ¹H NMR (CDCl₃): δ 01.49-1.77 (m, 2H, CH₂), 1.70 (br. s, ˜1H, OH), 1.85-3.0 (br. m, 10H, BH), 2.50-2.78 (m, 2H, CH₂), 3.48 (m, 1H, CH), 4.81 (br. s, 1H, OH), 6.60 (d, 2H, arom., J=8.8 Hz), 7.06 (d, 2H, arom., J=8.8 Hz), 7.14 (d, 2H, arom.), 7.19 (t, 1H, arom.), 7.28 (t, 2H, arom.). ¹³C NMR (CDCl₃): δ 32.68, 38.29, 72.35, 83.56, 86.01, 126.20, 128.52, 128.61, 128.68, 129.04, 141.12, 155.78. Accurate mass HRMS (ESI): m/z calcd for C₁₇H₂₅B₁₀O₂ (M−1)⁻ 369.2852, found 369.2851.

### Example 15

The procedure and conditions described for the synthesis of (RS)-1-[1-(4-hydroxyphenyl)-1,12-dicarba-closo-dodecaborane-12-yl]heptane-1-ol were adapted using 280 mg (0.63 mmol) (RS)-(2,3-dihydro-1H-inden-5-yl)-[1-(4-methoxyphenyl)-1,12-dicarba-closo-dodecaborane-12-yl]methanol as the starting material. Purification of the products is carried out by silica gel column chromatography (hexanes/EtOAc, 9/1, v/v) to yield a white solid. Further purification can be achieved by refluxing a suspension of the product in hexanes/i-propanol [24:1] and, after cooling the suspension to 0° C., washing the obtained residue with ice-cold pentane.

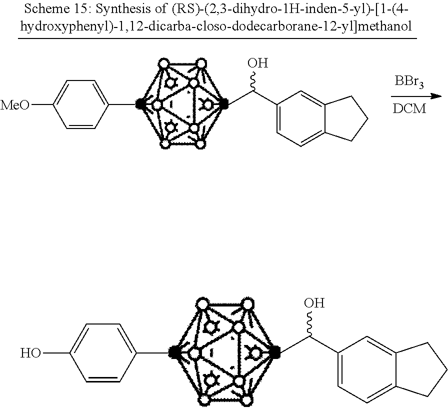

Yield: 240 mg (89%), R_{f}. 0.19 (hexanes/EtOAc, 9/1, v/v), m.p.: 231° C. (decomp.). ¹H NMR (Acetone-d₆): δ 1.9-3.0 (br. m, 10H, BH), 2.06 (m, ˜2H, CH₂), 2.88 (m, ˜4H, 2×CH₂), 4.68 (s, H, OH), 4.99 (m, 1H, CH), 6.66 (d, 2H, arom., J=8.6 Hz), 6.97 (d, 1H, arom.), 7.05 (d, 2H, arom., J=8.9 Hz), 7.08 (s, 1H, arom.), 7.13 (d, 2H, arom.), 8.51 (s, H, OH). ¹³C NMR (Acetone-d₆): δ 26.41, 33.09, 33.31, 75.96, 84.58, 88.01, 115.65, 123.59, 124.21, 125.86, 128.30, 129.09, 140.63, 144.24, 144.71, 158.58. Accurate mass HRMS (ESI): m/z calcd for C₁₅H₂₅B₁₀O₂ (M−1)⁻ 381.2852, found 381.2855.

### Example 16

To a solution of 1-[1-(4-methoxyphenyl)-1,12-dicarba-closo-dodecaborane-12-yl]heptane-1-one (630 mg, 1.74 mmol) in anhydrous DCM (40 mL) was added boron tribromide (5.2 mL, 5.2 mmol, 1 M solution in DCM) at 0° C. The reaction mixture was stirred at room temperature overnight, poured carefully into ice-cold 1 M HCl (60 mL) and extracted with DCM. The organic phase was washed with a 10% sodium thiosulfate solution and brine and dried over MgSO₄. The solvents were evaporated and the residue purified by silica gel column chromatography (hexanes/EtOAc, 9/1, v/v) to yield a white solid. Further purification can be achieved by recrystallization from pentane or hexanes (−20° C.).

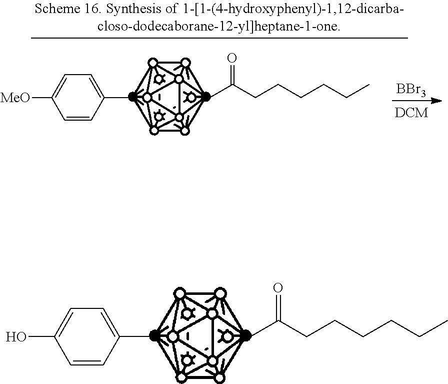

Yield: 520 mg (86%), R_{f}. 0.31 (hexanes/EtOAc, 9/1, v/v), m.p.: 79-80° C. ¹H NMR (CDCl₃): δ 0.86 (t, 3H, CH₃), 1.12-1.27 (m, 6H, 3×CH₂), 1.39-1.46 (m, 2H, CH₂), 1.55-3.40 (br. m, 10H, BH), 2.39 (t, 2H, C(O)—CH₂), 5.11 (br. s, 1H, OH), 6.62 (d, 2H, arom., J=8.7 Hz), 7.05 (d, 2H, arom., J=8.9 Hz). ¹³C NMR (CDCl₃): δ 14.09, 22.52, 23.53, 28.44, 31.54, 39.40, 83.61, 85.83, 114.95, 128.49, 128.87, 155.99, 195.87. Accurate mass HRMS (ESI): m/z calcd for C₁₅H₂₇B₁₀O₂ (M−1)⁻ 347.3001, found 347.3014.

### Example 17

The procedure and conditions described for the synthesis of (RS)-1-[1-(4-hydroxyphenyl)-1,12-dicarba-closo-dodecaborane-12-yl]heptane-1-ol were adapted using 300 mg (0.825 mmol) (S)-1-[1-(4-methoxyphenyl)-1,12-dicarba-closo-dodecaborane-12-yl]heptane-1-ol as the starting material. Purification of the products is carried out by silica gel column chromatography (hexanes/EtOAc, 9/1, v/v) to yield a white solid. Further purification can be achieved by refluxing a suspension of the product in hexanes/i-propanol [24:1] and, after cooling the suspension to 0° C., washing the obtained residue with ice-cold pentane. The enantiomeric excess (ee) was estimated to be >85% according to analysis of the ¹H-NMR spectrum of the corresponding Mosher ester. The absolute configuration was determined by analysis of the ¹H-NMR spectrum of the corresponding Mosher ester.

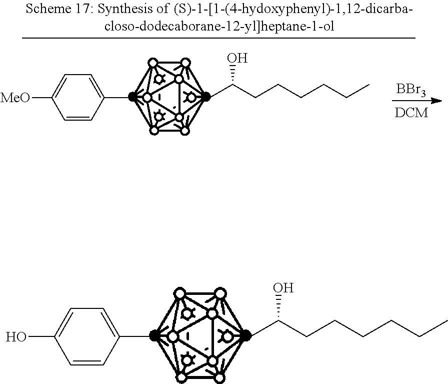

Yield: 220 mg (76%), R_{f}. 0.23 (hexanes/EtOAc, 9/1, v/v), m.p.: 120-121° C., [α]_{D}^{20° C.}=+23° (0.1, DCM). ¹H NMR (CDCl₃): δ 0.87 (t, 3H, CH₃), 1.15-1.30 (m, 8H, 4×CH₂), 1.39-1.45 (m, 2H, CH₂), 1.66-1.71 (m, ˜2H, OH & H₂O), 1.85-3.0 (br. m, 10H, BH), 3.46 (m, 1H, CH), 5.08 (br. s, 1H, OH), 6.61 (d, 2H, arom., J=8.8 Hz), 7.07 (d, 2H, arom., J=8.9 Hz). ¹³C NMR (CDCl₃): δ 14.19, 22.70, 26.58, 28.97, 31.81, 36.90, 73.16, 83.49, 86.33, 114.90, 128.67, 129.03, 155.84. Accurate mass HRMS (ESI): m/z calcd for C₁₅H₂₉B₁₀O₂ (M−1)⁻ 349.3165, found 349.3162.

### Example 18

The procedure and conditions described for the synthesis of (RS)-1-[1-(4-hydroxyphenyl)-1,12-dicarba-closo-dodecaborane-12-yl]heptane-1-ol were adapted using 300 mg (0.825 mmol) (R)-1-[1-(4-methoxyphenyl)-1,12-dicarba-closo-dodecaborane-12-yl]heptane-1-ol as the starting material. Purification of the products is carried out by silica gel column chromatography (hexanes/EtOAc, 9/1, v/v) to yield a white solid. Further purification can be achieved by refluxing a suspension of the product in hexanes/i-propanol [24:1] and, after cooling the suspension to 0° C., washing the obtained residue with ice-cold pentane. The enantiomeric excess (ee) was estimated to be >85% according to analysis of the ¹H-NMR spectrum of the corresponding Mosher ester. The absolute configuration was determined by analysis of the ¹H-NMR spectrum of the corresponding Mosher ester.

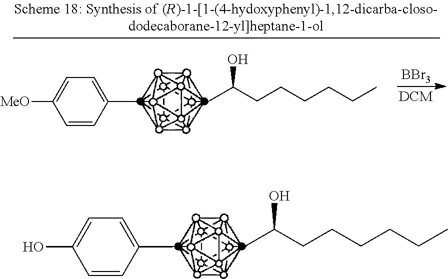

Yield: 180 mg (62%), R_{f}. 0.23 (hexanes/EtOAc, 9/1, v/v), m.p.: 120-121° C., [α]_{D}^{20° C.}=−28° (0.1, DCM). ¹H NMR (CDCl₃): δ 0.87 (t, 3H, CH₃), 1.15-1.30 (m, 8H, 4×CH₂), 1.39-1.45 (m, 2H, CH₂), 1.68-1.76 (m, ˜2H, OH & H₂O), 1.9-3.0 (br. m, 10H, BH), 3.47 (m, 1H, CH), 5.17 (br. s, 1H, OH), 6.61 (d, 2H, arom., J=8.8 Hz), 7.07 (d, 2H, arom., J=8.9 Hz). ¹³C NMR (CDCl₃): δ 14.19, 22.70, 26.58, 28.96, 31.81, 36.90, 73.17, 83.50, 86.31, 114.90, 128.67, 129.01, 155.86. Accurate mass HRMS (ESI): m/z calcd for C₁₅H₂₉B₁₀O₂ (M−1)⁻ 349.3165, found 349.3158.

### Example 19

Estrogen receptor beta (ERβ) agonists have the potential to function as tumor suppressors in the treatment of cancers, such as breast, colon, and prostate cancer. Such agents can also be used in the treatment of inflammatory diseases, such as arthritis and inflammatory bowel disease, as well as in some neurodegenerative and psychotropic disorders.

A library of twenty two compounds (Table 2) was synthesized (for example, as described above or using methods derived therefrom), and biologically evaluated in vitro for estrogen receptor beta (ERβ) selective agonist activity. The library of twenty two compounds was synthesized based on reference compounds (Table 1). Within synthesized structures (Table 2), the B and C rings of the endogenous ligand E2 were replaced with a carborane cluster. The hydrophobicity character and the spherical geometry of the carborane can play a role in enhancing the binding affinity of ligands to estrogen receptor.

In addition to the three reference compounds (Table 1) and the library of twenty two synthesized compounds (Table 2), three compounds described by Thirumamagal, B T S et al. (Bioconj. Chem. 2006, 17, 114-1150) were also included in the in vitro evaluation of ERβ selective agonist activity (Table 3).

The selectivity and potency of the various compounds was carried out via in vitro testing in ERα and ERβ cell-based reporter assays. The activity of the selected compounds was determined in the cell-based reporter assays in HEK293 cells. The HEK293 cell line was chosen as it does not express endogenous ERα or ERβ at significant levels.

The HEK293 cells were propagated in a monolayer in phenol red-free DMEM supplemented with 10% fetal bovine serum, 2 mM Glutamax and penicillin/streptomycin (Thermo Fisher Scientific, MA, USA) and incubated in a 5% CO₂ humidified atmosphere at 37° C. Right before transfection, the growth medium was changed to phenol red-free DMEM supplemented with 4% HyClone Fetal Bovine Serum, Charcoal/Dextran Treated (GE Healthcare Life Sciences, USA) and 2 mM Glutamax (starvation medium). The cells were transfected with the expression vector encoding human full-length ERα or ERβ and with the reporter vector containing 3 repeats of estrogen responsive elements (ERE) followed by the minimal thymidine kinase promoter from the herpes simplex virus in the pGL4 vector (Promega, USA). Luciferase served as a reporter gene. The transfection was carried out in 10 cm dishes (Nunc) in the starvation medium. After 24 hours, the cells were trypsinized, counted and seeded to cell culture treated, white, solid 1536-well plates (Corning Inc., NY, USA) at 1500 cells/well in 4 μl of total media volume. The compounds to be tested were diluted in DMSO and transferred to the cells using an acoustic dispenser Echo 520 (Labcyte). The compounds were tested at least at 12 different concentration points in the range from 10 pM to 100 pM, in triplicates. Luciferase activity was determined after 24 hours of incubation with compounds with Britelite plus luciferase reporter gene assay reagent (Perkin Elmer, USA), according to the manufacturer protocol. The luciferase signal was measured on an Envison multimode plate reader (Perkin Elmer, USA). Data were collected and processed using an in-house built LIMS system ScreenX and GraphPad Prism software. EC₅₀ values were calculated using a regression function (dose response, variable slope). The assay description is summarized in Table 4.

The results of the in vitro evaluation of the compounds for estrogen receptor beta (ERβ) selective agonist activity are summarized in Table 5. Experiments on compound 04 indicated it had an EC₅₀ at ERα of >5000 nM and an EC₅₀ at ERβ of 46 nM, indicating a high ERβ selectivity. Experiments on compound 05 indicated it had an EC₅₀ at ERα of >5000 nM and an EC₅₀ at ERβ of 64 nM, indicating a high ERβ selectivity.

The results (Table 5) indicated that the active carboranyl compounds of the synthesized library were those where the para-hydrophenyl-ring (A-ring) of E2 was retained to allow for hydrogen bond- and pi-stacking interactions with the receptor. The results further indicated that the active compounds from the synthesized library were those where the D-ring of E2, containing a 17β-hydroxyl group, was replaced with an alkyl- or a 1-hydroxyalkyl group. The latter structural element appeared to be related to selectivity for ERβ.

One promising compound of this library was 1-(4-hydoxyphenyl)-12-(1-hydroxyheptyl)-1,12-dicarba-closo-dodecaborane (06). Evaluation of this compound in a luciferase reporter-based cell assay in human embryonic kidney (HEK) cells (Sedlak, D. et al. Comb. Chem. High T. Scr. 2011, 14, 248-266) resulted in an EC₅₀ of 5 nM at ERβ and an ERβ-to-ERα agonist ratio of 1,800. For comparison, the standard ERβ selective agonist diarylpropionitrile (DPN) had an EC₅₀ of 6.3 nM and an ERβ-to-ERα agonist ratio of 358.

TABLE 1
Reference Compounds.

| Compound Name | Structure |
|---|---|
| Estradiol (E2) | 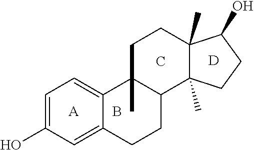 |
| Diarylpropionitrile (DPN) (ERβ selective agonist) | 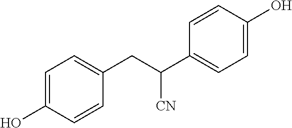 [SMILES: N#CC(Cc1ccc(O)cc1)c1ccc(O)cc1] |
| Propyl pyrazole triol (PPT) (ERα selective agonist) | 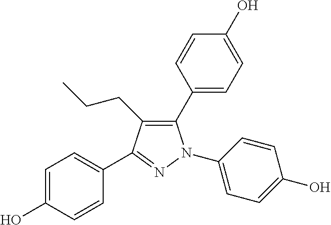 [SMILES: CCCc1c(-c2ccc(O)cc2)nn(-c2ccc(O)cc2)c1-c1ccc(O)cc1] |

TABLE 2
Synthesized library of compounds.

| Compound | Structure |
|---|---|
| 04 | 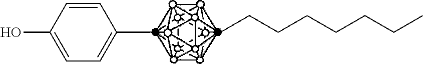 |
| 05 | 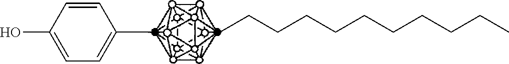 |
| 06 | 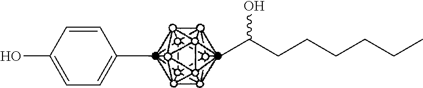 |
| 07 | 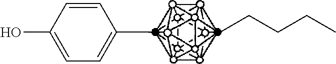 |
| 08 | 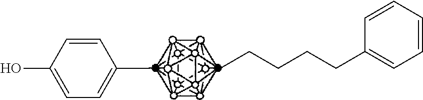 |
| 09 | 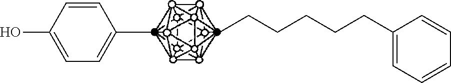 |
| 10 | 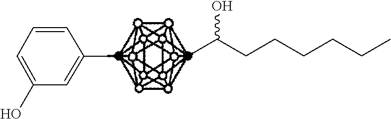 |
| 11 | 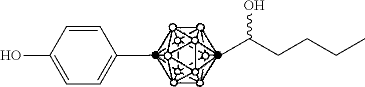 |
| 12 | 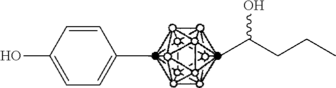 |
| 13 | 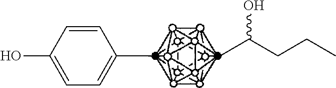 |
| 14 | 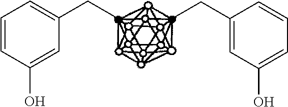 |
| 15 | 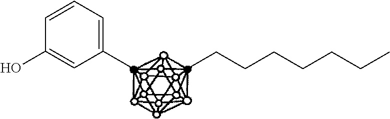 |
| 16 | 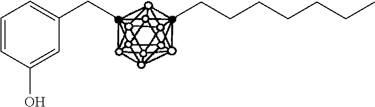 |
| 17 | 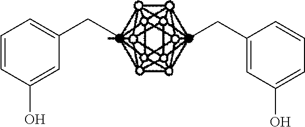 |
| 18 | 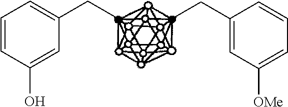 |
| 19 | 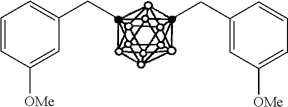 |
| 20 | 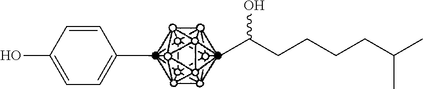 |
| 21 | 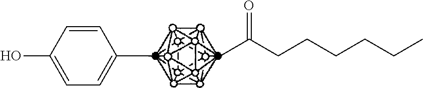 |
| 22 | 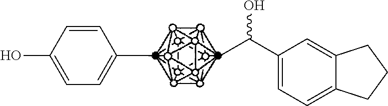 |
| 23 | 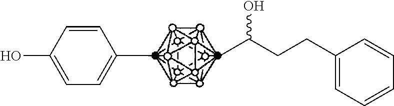 |
| 24 | 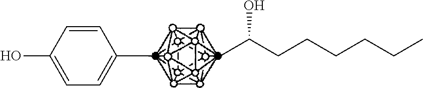 |
| 25 | 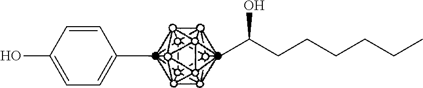 |

TABLE 3
Compounds from Thirumamagal BTS et al. Bioconj. Chem. 2006, 17, 114-150.

| Compound | Structure |
|---|---|
| 01 | 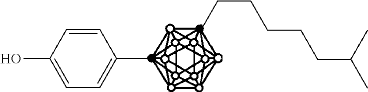 |
| 02 | 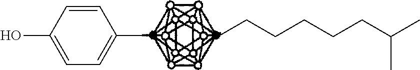 |
| 03 | 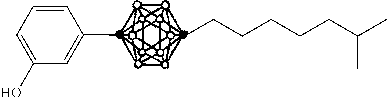 |

TABLE 4
Assay description

| | Reporter | | | | |
|---|---|---|---|---|---|
| assay | mode | Steroid receptor | Reporter vector | Cells | Genetic modification |
| ERα | Agonist | Human full-length ERα | pGL4-3xERE-Luc2 | HEK293 | Transiently transfected cells |
| ERβ | Agonist | Human full-length ERβ | pGL4-3xERE-Luc2 | HEK293 | Transiently transfected cells |
| AR | Agonist | Human full-length AR | pGL4-MMTV-Luc2 | U2OS | Stable transfectants, clone 22 |
| AR | Antagonist | Human full-length AR | pGL4-MMTV-Luc2 | U2OS | Stable transfectants, clone 22 |
| GR | Agonist | Human full-length GR | pGL4-MMTV-Luc2 | U2OS | Stable transfectants, clone 26 |
| GR | Antagonist | Human full-length GR | pGL4-MMTV-Luc2 | U2OS | Stable transfectants, clone 26 |
| Viability | — | — | — | HEK293 | — |

| | Cell | | | |
|---|---|---|---|---|
| | incubation | | | |
| | with | | | |
| assay | compounds | Antagonist mode | Assay readout | Assay reagent |
| ERα | 24 hours | — | Luciferase (luminescence) | Britelite Plus (Perkin Elmer) |
| ERβ | 24 hours | — | Luciferase (luminescence) | Britelite (Perkin Elmer) |
| AR | 24 hours | | Luciferase (luminescence) | Britelite (Perkin Elmer) |
| AR | 24 hours | 2 nM Dihydrotestosterone | Luciferase (luminescence) | Britelite (Perkin Elmer) |
| GR | 24 hours | | Luciferase (luminescence) | Britelite (Perkin Elmer) |
| GR | 24 hours | 10 nM Dexamethasone | Luciferase (luminescence) | Britelite (Perkin Elmer) |
| Viability | 24 hours | | Luciferase (luminescence) | ATPlite 1step (Perkin Elmer) |

TABLE 5
Results of in vitro testing of the compounds in ERα and ERβ cell-based reporter assays.

| | | ERα | | | | | ERβ | | | | | |
|---|---|---|---|---|---|---|---|---|---|---|---|---|
| | | | | EC₅₀ | | | | | EC₅₀ | | | ERβ |
| compound | | Log(EC₅₀) | SD | (nM) | Efficacy | SD | Log(EC₅₀) | SD | (nM) | Efficacy | SD | Selectivit |
| E2 | | −10.16 | 0.16 | 0.07 | 99 | 6.1 | −10.58 | 0.1 | 0.03 | 93 | 2.7 | 2.7 |
| DPN | | −5.78 | 0.11 | 1668 | 95 | 8.7 | −8.88 | 0.0 | 1.33 | 113 | 2.4 | 1252 |
| PPT | | −8.48 | 0.13 | 3.3 | 101 | 7.2 | low | | low | | | |
| 01 | | −5.11 | 0.02 | 7810 | 80 | 1.7 | low | | low | | | |
| 02 | Trial 1 | −5.49 | 0.08 | 3268 | 120 | 7.9 | −7.48 | 0.0 | 33 | 110 | 2.5 | 99 |
| | Trial 2 | −5.17 | 0.01 | 6684 | 93 | 1.3 | −6.56 | 0.0 | 277 | 95 | 4.3 | 24 |
| 03 | | −4.20 | 1.98 | 92635 | | | −5.56 | 0.0 | 2780 | 64 | 4.5 | |
| 04 | Trial 1 | −4.46 | 0.03 | 35000 | 57 | 2.7 | −5.89 | 0.0 | 1292 | 47 | 0.4 | |
| | Trial 2 | −5.90 | 0.11 | 1251 | 113 | 9.7 | −7.10 | 0.0 | 80 | 96 | 3.4 | 16 |
| 05 | | low | | low | | | −5.22 | 0.0 | 5997 | 77 | 2.6 | |
| 06 | Trial 1 | −5.78 | 0.31 | 1647 | 30 | 9.4 | −7.71 | 0.0 | 19 | 100 | 3.5 | 85 |
| | Trial 2 | −5.51 | 0.02 | 3092 | 66 | 1.9 | −7.76 | 0.0 | 17 | 103 | 4.0 | 177 |
| 07 | | −7.12 | 0.03 | 75 | 104 | 3.0 | −8.02 | 0.1 | 10 | 90 | 5.0 | 7.8 |
| 08 | | −4.75 | 1.09 | 17985 | | | −7.05 | 0.0 | 90 | 68 | 3.1 | 200 |
| 09 | | −4.58 | 2.61 | 26034 | | | −6.60 | 0.0 | 253 | 55 | 2.0 | 103 |
| 10 | | low | | low | | | −6.76 | 0.0 | 175 | 55 | 3.2 | >571 |
| 11 | | −6.77 | 0.10 | 168 | 96 | 7.8 | −8.57 | 0.0 | 2.7 | 86 | 2.9 | 62 |
| 12 | Trial 1 | −7.45 | 0.08 | 36 | 101 | 4.8 | −8.89 | 0.1 | 1.3 | 85 | 4.1 | 28 |
| | Trial 2 | −7.53 | 0.09 | 30 | 94 | 5.3 | −8.94 | 0.0 | 1.14 | 104 | 2.8 | 26 |
| 13 | | −7.13 | 0.07 | 74 | 94 | 4.3 | −8.86 | 0.1 | 1.4 | 97 | 4.1 | 54 |
| 14 | | low | | low | | | low | | low | | | |
| 15 | | low | | low | | | low | | low | | | |
| 16 | | low | | low | | | low | | low | | | |
| 17 | | low | | low | | | low | | low | | | |
| 18 | | low | | low | | | low | | low | | | |
| 19 | | low | | low | | | low | | low | | | |
| 20 | | −4.89 | 0.15 | 12815 | | | −7.46 | 0.0 | 35 | 81 | 2.6 | 368 |
| 21 | | −5.55 | 0.04 | 2808 | 54 | 2.7 | −6.82 | 0.0 | 151 | 66 | 2.5 | 19 |
| 22 | | −6.07 | 0.07 | 857 | 65 | 6.0 | −7.54 | 0.0 | 29 | 32 | 1.9 | 30 |
| 23 | | −5.96 | 0.01 | 1096 | | | −7.45 | 0.0 | 36 | 75 | 1.5 | 31 |
| 24 | | −5.00 | 0.11 | 10112 | | | −7.40 | 0.0 | 40 | 91 | 1.9 | 253 |
| 25 | | −5.51 | 0.04 | 3114 | | | −7.54 | 0.0 | 29 | 83 | 2.5 | 108 |

Trial 1, Trial 2 = for compounds with multiple trials reported, Trial 2 data is believed to be more reliable, but all data reported here for
Low = activity detected, but activity was so low that exact value not reported.
>, < = exact value could not be determined from the tested concentration range.

### Example 20

The family of steroid receptors consists of six highly evolutionary conserved, but structurally related receptors. Natural ligands for steroid receptors are structurally even more related and despite their high similarity, they can bind very selectively to their dedicated target. For example, cortisol is the ligand of the glucocorticoid receptor and it does not interact with estrogen receptors.

As discussed above, the library of carborane derivatives shows preferential activation of ERβ over ERα, based on profiling over a wide concentration range. It is however possible that these carborane derivatives, being a new class of artificially prepared ERβ ligands and structurally unrelated to the natural estrogen hormones, can have a different activity profile and can interact with the remaining members of the steroid receptor family, such as with androgen receptor. Such unwanted activity would have profound biological consequences.

To evaluate the off-target activities of the carborane compounds on other steroid receptors, androgen receptor (AR) and glucocorticoid receptor (GR) cell-based luciferase reporter assays were performed in the same manner as the estrogen receptor (ER) reporter assays described above (Sedlak, D. et al. Comb. Chem. High T. Scr. 2011, 14, 248-266). The compounds tested were E2, DPN, PPT, 01, 02, 03, 04, 05, 06, 07, 08, 09, 10, 11, 12, 13, 20, 21, 22, 23, 24, and 25. The AR and GR assay descriptions are summarized in Table 4. The assays were carried out with stable reporter cell lines expressing full-length AR or GR in the osteosarcoma U2OS cell line with no endogenous expression of these receptors. The experiment was performed in the agonist and antagonist mode to detect all possible interactions of compounds with the receptor. In the antagonist mode, dihydrotestosterone (DHT) or dexamethasone was added to the cell culture 1 hour after the compound addition to the final concentration of 2 nM or 10 nM, for the AR and GR reporter assay, respectively. In the concentration range tested (100 μM to 100 μM), no agonistic or antagonistic activities on AR or GR were detected for the tested compounds, suggesting that the activity of carborane derivatives is restricted to ERβ only.

### Example 21

The in vitro cytotoxicity of the compounds was assessed by running a viability assay on HEK293 cells parallel to the ERα and ERβ reporter assays to ensure the comparability of the obtained results. The non-transfected HEK293 cells were seeded to the 384-well plates at 5000 cell/well, compounds were added and the timing of all subsequent steps was exactly the same as in the reporter assays. The compounds tested were E2, DPN, PPT, 01, 02, 03, 04, 05, 06, 07, 08, 09, 10, 11, 12, 13, 20, 21, 22, 23, 24, and 25. After 24 h of compound incubation with cells, the viability of cells was measured by determining the ATP level in the samples using luciferase cell viability assay, ATPlite 1step (Perkin Elmer, USA). The results are summarized in Table 6 and show that the compounds are non-toxic or they show a marginal cytotoxicity at the highest concentrations tested (IC₅₀>20 μM).

TABLE 6
Results of the in vitro cytotoxicity of the
compounds in the HEK293 viability assay.

| | HEK293 viability |
|---|---|
| Compound | IC₅₀ (μM) |

| | | |
|---|---|---|
| E2 | | Low |
| DPN | | Low |
| PPT | | Low |
| 01 | | 37 |
| 02 | Trial 1 | Low |
| | Trial 2 | 42 |
| 03 | | 84 |
| 04 | Trial 1 | 25 |
| | Trial 2 | 45 |
| 05 | | Low |
| 06 | Trial 1 | 18 |
| | Trial 2 | 17 |
| 07 | | 33 |
| 08 | | 33 |
| 09 | | 35 |
| 10 | | 16 |
| 11 | | 34 |
| 12 | Trial 1 | 34 |
| | Trial 2 | 32 |
| 13 | | 47 |
| 20 | | 16 |
| 21 | | 24 |
| 22 | | 21 |
| 23 | | 18 |
| 24 | | 20 |
| 25 | | 19 |

Trial 1, Trial 2 = for compounds with multiple trials reported, Trial 2 data is believed to be more reliable, but all data reported here for completeness.
Low = activity detected, but activity was so low that exact value not reported.

### Example 22

A second library of compounds including (i) carboranes substituted with heteroaryl groups; (ii) carboranes comprising sulfide (thioether), sulfoxide, and sulfone groups; and (iii) carborane analogs was synthesized, and biologically evaluated in vitro for estrogen receptor beta (ERβ) selective agonist activity.

TABLE 7
Synthesized library of compounds.

| Compound | Structure |
|---|---|
| 26 | 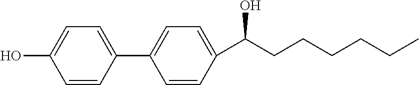 [SMILES: CCCCCC[C@H](O)c1ccc(-c2ccc(O)cc2)cc1] |
| 27 | 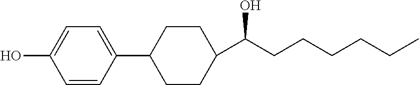 [SMILES: CCCCCC[C@H](O)C1CCC(c2ccc(O)cc2)CC1] |
| 28 | 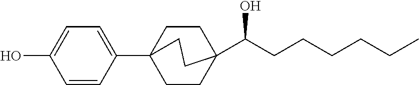 [SMILES: CCCCCC[C@H](O)C12CCC(c3ccc(O)cc3)(CC1)CC2] |
| 29 | 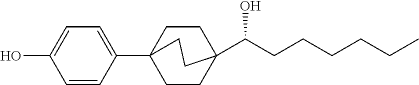 [SMILES: CCCCCC[C@@H](O)C12CCC(c3ccc(O)cc3)(CC1)CC2] |
| 30 | 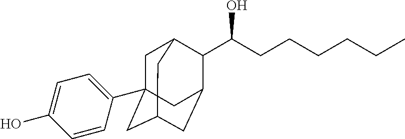 [SMILES: CCCCCC[C@H](O)C1C2CC3CC1CC(c1ccc(O)cc1)(C3)C2] |
| 31 | 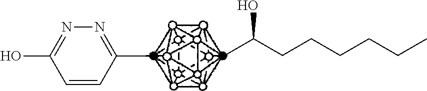 |
| 32 | 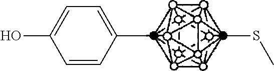 |
| 33 | 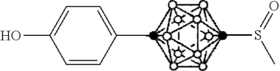 |
| 34 | 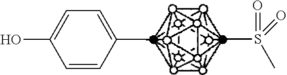 |
| 35 | 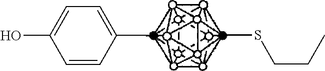 |
| 36 | 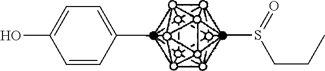 |
| 37 | 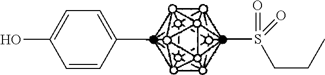 |
| 38 | 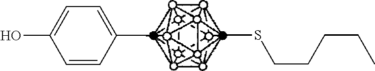 |
| 39 | 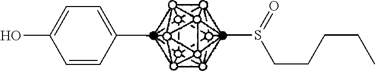 |
| 40 | 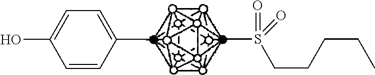 |
| 41 | 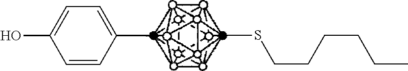 |
| 42 | 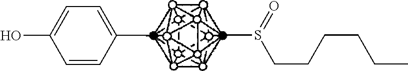 |
| 43 | 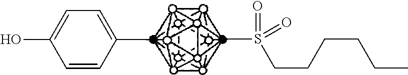 |
| 44 | 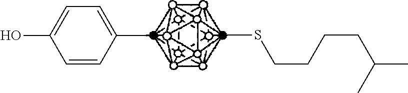 |
| 45 | 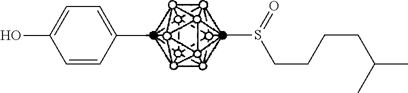 |
| 46 | 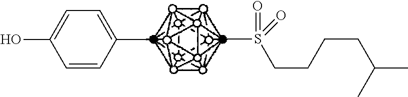 |
| 47 | 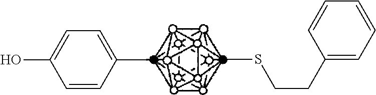 |
| 48 | 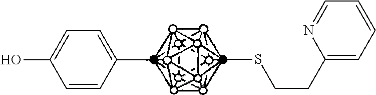 |
| 49 | 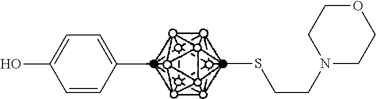 |
| 50 | 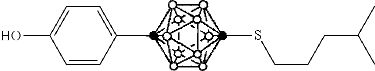 |
| 51 | 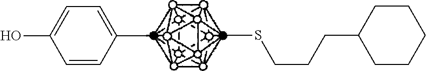 |
| 52 | 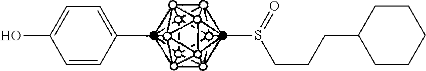 |
| 53 | 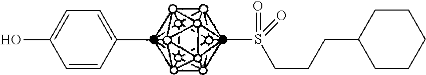 |
| 54 | 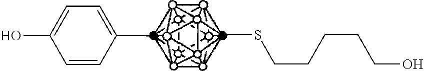 |
| 55 | 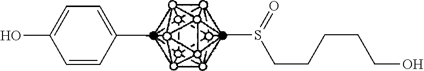 |
| 56 | 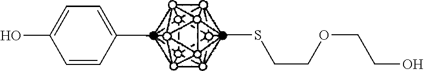 |
| 57 | 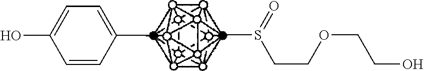 |
| 58 | 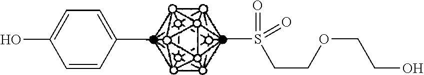 |
| 59 | 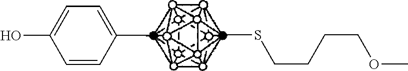 |
| 60 | 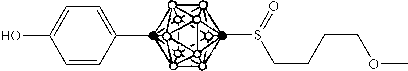 |
| 61 | 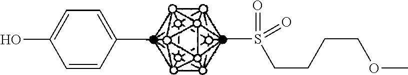 |

Compounds in the second library were prepared as described below.

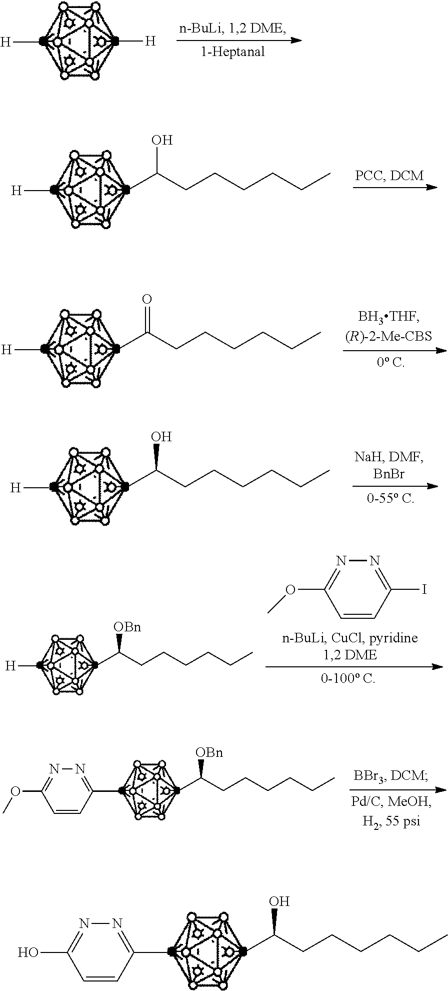

### Synthesis of 1-(Heptan-1-yl)-1,12-dicarba-closo-dodecaborane

To a solution of 1,12-dicarba-closo-dodecaborane (1.44 g, 10 mmol) in 1,2 dimethoxyethane (50 ml) was added dropwise a n-BuLi solution (2.5M in hexane, 4.4 ml) at 0° C. under Ar. The mixture was stirred at room temperature for 1 hour followed by addition of 1-heptanal (1.55 ml, 11 mmol) at 0° C. The mixture was stirred at room temperature overnight, and then was poured into 1 M HCl aqueous solution (100 ml), extracted with ethyl acetate (3×25 ml). The combined organic phases were washed with brine and dried over MgSO₄. The solvents were evaporated and the residue purified by Teledyne Isco (RediSepRf column) to yield a colorless oil. Yield 1.4 g. ¹H NMR (CDCl₃) δ 3.38-3.45 (m, 1H), 3.35-1.14 (m, 22H), 0.88 (t, 3H), MS 258.291.

### Synthesis of 1-(Heptan-1-one)-1,12-dicarba-closo-dodecaborane

Pyridinium chlorochromate (PCC, 1.7 g, 7.71 mmol) was suspended in anhydrous DCM (50 ml). A solution of 1-(heptan-1-yl)-1,12-dicarba-closo-dodecaborane (1.3 g, 5.04 mmol) in DCM (10 ml) was then added, and the reaction mixture was stirred at room temperature overnight. Diethyl ether (50 ml) was added and followed by molecular sieves, and then stirred for 1 h. The supernatant was decanted and the insoluble residue was washed with dry ether (3×20 ml). The combined organic phases were passed through a short column of Celite followed by evaporation. The residue was purified by Teledyne Isco (RediSepRf column) to yield a colorless oil, yield 1.2 g. ¹H NMR (CDCl₃) δ 1.16 (m, 21H), 0.88 (t, 3H), MS 256.189.

### Synthesis of (R)-1-(Heptan-1-yl)-1,12-dicarba-closo-dodecaborane

Borane-tetrahydrofuran complex (51 mL, 51 mmol, 1.0 M solution in THF, stabilized with 0.005 M N-isopropyl-N-methyl-tert-butylamine (NEVIBA)) followed by (R)-2-methyl-CBS-oxazaborolidine [(2-MeCBS] (5.1 mL, 5.1 mmol, 1.0 M solution in toluene) were added to 50 mL anhydrous THF. The reaction mixture was stirred at room temperature for 15 minutes and 1-(Heptan-1-one)-1,12-dicarba-closo-dodecaborane (1.3 g, 5.08 mmol) in 25 mL of anhydrous THF was added slowly over a period of 2 h at 0° C. The reaction mixture was stirred overnight at room temperature and then carefully quenched by addition of 2.0 M HCl (80 mL) in small portions to control H₂ development. Diethyl ether (100 mL) was added and the organic phase was washed brine and saturated NaHCO₃. The organic phase was dried over MgSO₄, filtered, and evaporated. The residue was purified by Teledyne Isco (RediSepRf column) to yield a colorless oil, yield 1.1 g 81%. ¹H NMR (CDCl₃) δ 3.38-3.45 (m, 1H), 3.35-1.14 (m, 22H), 0.88 (t, 3H), MS 258.291.

### Synthesis of (R)-1-(1-Benzyloxy)heptyl)-1,12-dicarba-closo-dodecaborane

To a solution of (R)-1-(Heptan-1-yl)-1,12-dicarba-closo-dodecaborane (900 mg, 3.49 mmol) in anhydrous DMF (10 ml), NaH (60% in mineral, 175 mg, 4.36 mmol) was added in one portion at 0° C., and then stirred at same temperature for 30 min. BnBr (746 mg, 4.36 mmol) was added, the reaction mixture was stirred at 55° C. for 3 h, cooled down room temperature and methanol (0.5 ml) was added slowly, diluted with ethyl acetate (50 ml), washed with water, brine and dried with Na₂SO₄. Solvents were evaporated and the residue was purified by Teledyne Isco (RediSepRf column) to yield a colorless oil, yield 1.1 g 93%. ¹H NMR (CDCl₃) δ 7.28 (d, 2H), 7.73 (d, 2H), 4.63 (d, 1H), 3.76 (s, 3H), 2.61-3.62 (m, 5H), 2.53 (s, 3H), 1.50-2.45 (m, 5H), MS calc. 329.200, Obsv. 329.189.

### Synthesis of (R)-1-(1-(6-Methoxypyridazin-3-yl)-12-(1-benzyloxy)heptyl)_{1,12}-dicarba-closo-dodecaborane

To a solution of (R)-1-(1-Benzyloxy)heptyl)-1,12-dicarba-closo-dodecaborane (106 mg, 0.19 mmol) in 1,2-dimethoxyethane (5 ml) was added dropwise a n-BuLi solution (2.5 M in hexane 92 μl, 0.23 mmol) at 0° C. under Ar. The mixture was stirred at room temperature for 1 h, and CuCl (46 mg, 0.23 mmol) was added in one portion. Stirring was continued at room temperature for 1 h, then pyridine (218 μl) was added, and 3-iodo-6-methoxypyridazine (35 mg, 0.23 mmol) was further added in one portion, and the mixture was heated at 80° C. for 48 h. After cooling, the reaction mixture was diluted with Et₂O and stirred at room temperature for 3 h. Insoluble materials were filtered through Celite. The filtrate was washed with Na₂S₂O₃, H₂O, and brine, dried over Na₂SO₄, then concentrated, and the residue was purified by Teledyne Isco (RediSepRf column) to yield pure product.

### Synthesis of (R)-1-(1-(6-Hydroxypyridazin-3-yl)-12-(1-benzyloxy)heptyl)_{1,12}-dicarba-closo-dodecaborane

To a solution of (R)-1-(1-(6-Methoxypyridazin-3-yl)-12-(1-benzyloxy)heptyl)_{1,12}-dicarba-closo-dodecaborane (35 mg, 0.08 mmol) in CH₂Cl₂ (1 ml) was added dropwise a 1 M solution of BBr₃ in CH₂Cl₂ (0.28 ml) at 0° C. The mixture was stirred at room temperature for 2 h, then poured into ice water, and extracted with CH₂Cl₂. The organic layer was washed with brine, dried over Na₂SO₄, and concentrated. Purification by Teledyne Isco (RediSepRf column) to yield pure product, yellow solid. ¹H NMR (CDCl₃) δ 7.27 (d, 2H), 6.82 (d, 2H), 3.26 (d, 1H), 1.50-3.1 (m, 22H) 0.88 (t, 3H), MS calc. 441.351, Obsv. 441.362.

### Synthesis of (R)-1-[1-(6-Hydroxypyridazin-3-yl)-1,12-dicarba-closo-dodecaborane-12-yl]heptane-1-ol

The mixture of (R)-1-(1-(6-Hydroxypyridazin-3-yl)-12-(1-benzyloxy)heptyl)_{1,12}-dicarba-closo-dodecaborane (26 mg, 0.06 mmol), Pd/C oncarbon (5 mg) in methanol (5 ml) was reacted with H₂ in Parr shaker under 55 psi for 48 h. Filtered, washed with methanol, the combined filtrates were concentrated and the residue was purified by Teledyne Isco (RediSepRf column) to yield pure product, brown solid. ¹H NMR (CDCl₃) δ 7.27 (d, 2H), 6.82 (d, 2H), 3.26 (d, 1H), 1.50-3.1 (m, 22H) 0.88 (t, 3H), MS calc. 351.310, Obsv. 351.310.

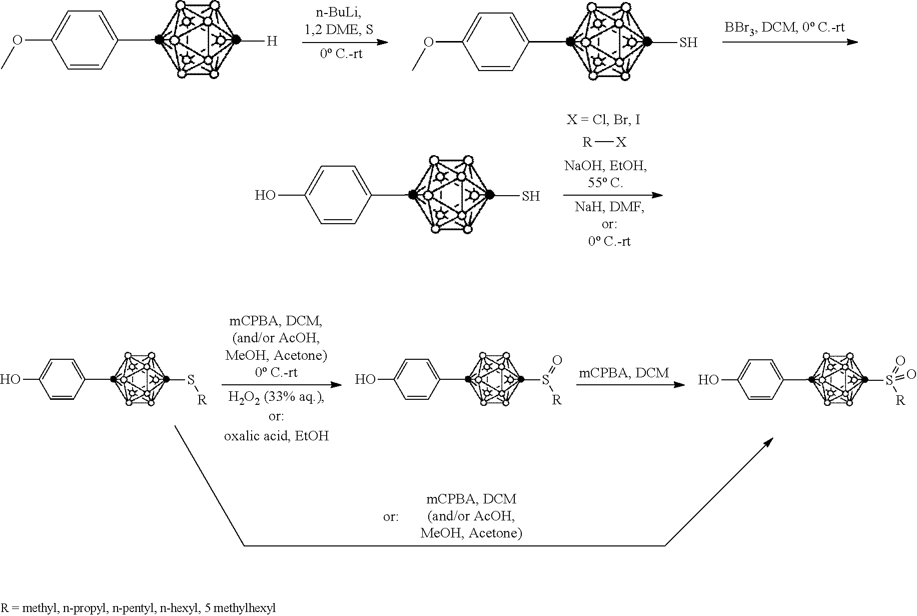

### Synthesis of 1-mercapto-12-(4-methoxyphenyl)-1,12-dicarba-closododecaborane

To a solution of 1-(4-Methoxyphenyl)-1,12-dicarba-closo-dodecaborane (1.58 g, 6.3 mmol) in 1,2 dimethoxyethane (50 ml) was added dropwise a n-BuLi solution (2.5 M in hexane, 2.8 ml) at 0° C. under Ar. The mixture was stirred at room temperature for 1 hour followed by addition of elemental sulfur (250 mg, 7.8 mmol) at 0° C. The mixture was stirred at room temperature 3 h, and 50 ml of water were added. The organic layer was separated and then extracted by 50 ml of 10% aqueous NaOH. The aqueous layer was combined with the extract and the mixture acidified with HCl to a pH of ca. 1. The product was extracted twice with 100 ml of diethyl ether; the organic phases were dried over Na₂SO₄. Solvents were evaporated and the residue was purified by Teledyne Isco (RediSepRf column) to yield pure product, 1-mercapto-12-(4-methoxyphenyl)-1,12-dicarba-closododecaborane yellow solid, 1.53 g, as yellow solid.

### Synthesis of 1-mercapto-12-(4-hydroxyphenyl)-1,12-dicarba-closododecaborane

To a solution of 1-mercapto-12-(4-methoxyphenyl)-1,12-dicarba-closododecaborane 1.5 g (5.3 mmol) in CH₂Cl₂ (20 ml) was added 1 M solution of BBr₃ in CH₂Cl₂ (20 ml) at 0° C. The mixture was stirred at room temperature for 16 h, then poured into ice water, and extracted with CH₂Cl₂. The organic layer was washed with brine, dried over Na₂SO₄, and concentrated. Purification by Teledyne Isco (RediSepRf column) to yield pure product, 1-mercapto-12-(4-hydroxyphenyl)-1,12-dicarba-closododecaborane 1.17 gas white solid.

### Synthesis of 1-Methylthio-12-(4-hydroxyphenyl)-1,12-dicarba-closo-dodecaborane

To a solution of 1-mercapto-12-(4-hydroxyphenyl)-1,12-dicarba-closododecaborane (112 mg, 0.42 mmol) in ethanol (10 ml) was added NaOH (34 mg, 0.84 mmol), the reaction mixture was stirred at 55° C. for 15 minutes before iodomethane (60 mg, 0.42 mmol) was added. The final reaction mixture was stirred at 55° C. overnight, cooled to room temperature and adjusted to pH 1 to 3. Ethanol was removed and the residue was dissolved in ethyl acetate and washed with brine, the organic layer was dried over Na₂SO₄ concentrated in vacuo and the residue was purified by silica gel column. Pure product 1-methylthio-12-(4-hydroxyphenyl)-1,12-dicarba-closo-dodecaborane, 100 mg (yield, 85%) was obtained as a yellowish solid. ¹H NMR (CDCl₃) δ 7.04 (d, 2H), 6.60 (d, 2H), 2.15 (s, 3H), 1.16-3.62 (m, 11H), MS (−ESI) calc. 281.392 (M−1), Obsv. 281.200.

Alternative General S-Alkylation Procedure:

To a suspension of Sodium Hydride (60% dispersion in mineral oil, 2.1 or 3.1 equivalents) in DMF at 0° C. was added a solution of 1-mercapto-12-(4-hydroxyphenyl)-1,12-dicarba-closododecaborane (1.0 equivalents) in DMF. The resulting mixture was stirred until effervescence ceased. A solution of the alkyl halide (0.95 equivalents) in DMF was added dropwise to this mixture over several minutes at 0° C. (In the case of alkyl chlorides, catalytic sodium iodide was added thereafter.) The final reaction mixture was stirred at room temperature from 1 h to overnight, quenched with H₂O, and adjusted to pH 2 with 2N HCl. The aqueous layer was extracted 3× with ether or ethyl acetate, the organic layer was washed with H₂O (4×), and brine (1×), dried over Na₂SO₄ and concentrated in vacuo. The residue was purified by CombiFlash Teledyne Isco (RediSepRf column).

### Synthesis of 1-Methylsulfinyl-12-(4-hydroxyphenyl)-1,12-dicarba-closo-dodecaborane

To a solution of 1-methylthio-12-(4-hydroxyphenyl)-1,12-dicarba-closo-dodecaborane (34 mg, 0.12 mmol) in EtOH (2 ml), hydrogen peroxide (33%, 90 μl) was added followed by oxalic acid (11.4 mg, 0.12 mmol). The final reaction mixture was stirred at room temperature for 48 h, diluted with ethyl acetate (20 ml), washed with water, NaHCO₃, and brine, dried over Na₂SO₄ and concentrated in vacuo. The residue was purified by silica gel column to afford 1-methylsulfinyl-12-(4-hydroxyphenyl)-1,12-dicarba-closo-dodecaborane, 18 mg (yield 50%), as an off white solid. ¹H NMR (CDCl₃) δ 7.05 (d, 2H), 6.64 (d, 2H), 3.54 (s, 3H), 2.55-3.62 (m, 5H), 1.16-2.53 (m, 6H), MS (−ESI) calc. 297.1948 (M−1), Obsv. 297.1947.

Alternative Sulfoxide Formation Procedure:

To a solution of sulfide (1.0 equivalents) in dichloromethane (0.1M) at 0° C. was added dropwise a solution of mCPBA (77%, 1.0 equivalents) in dichloromethane (0.1M). note: in select cases, a co-solvent such as AcOH, MeOH, or acetone can be used. The reaction mixture was stirred at 0° C. for 1 h. The reaction mixture was then diluted with dichloromethane, washed with NaS₂O₃, NaHCO₃and brine, dried over Na₂SO₄, and concentrated in vacuo. Alternatively, the reaction can be dried with a gentle stream of argon and then the same workup procedure can be carried out with ethyl acetate instead. The residue was purified by CombiFlash Teledyne Isco (RediSepRf column).

Sulfone Formation Procedure:

To a solution of sulfoxide (1.0 equivalents) in dichloromethane (0.1M) was added mCPBA (77%, 1.0-2.0 equivalents). note: in select cases, a co-solvent such as AcOH, MeOH, or acetone can be used. The reaction mixture was stirred for 1 h to overnight. The reaction mixture was then diluted with dichloromethane, washed with NaS₂O₃, NaHCO₃and brine, dried over Na₂SO₄, and concentrated in vacuo. Alternatively, the reaction can be dried with a gentle stream of argon and then the same workup procedure can be carried out with ethyl acetate instead. The residue was purified by CombiFlash Teledyne Isco (RediSepRf column).

### Synthesis of 1-Methylsulfonyl-12-(4-hydroxyphenyl)-1,12-dicarba-closo-dodecaborane

To a solution of 1-methylthio-12-(4-hydroxyphenyl)-1,12-dicarba-closo-dodecaborane (35 mg, 0.12 mmol) in DCM (2 ml), mCPBA (63 mg, 0.36 mmol) was added. The reaction mixture was stirred at room temperature for 4 h, washed with Na₂S₂O₃, NaHCO₃and brine, dried over Na₂SO₄, concentrated in vacuo. The residue was purified by silica gel column to afford 1-methylsulfonyl-12-(4-hydroxyphenyl)-1,12-dicarba-closododecaborane, 31 mg (yield 80%), as an off-white solid. ¹H NMR (CDCl₃) δ 7.02 (d, 2H), 6.62 (d, 2H), 2.93 (s, 3H), 2.95-3.62 (m, 3H), 1.16-2.92 (m, 8H), MS (−ESI) calc. 313.1896 (M−1), Obsv. 313.1896.

### Synthesis of 1-Propylthio-12-(4-hydroxyphenyl)-1,12-dicarba-closo-dodecaborane

1-Propylthio-12-(4-hydroxyphenyl)-1,12-dicarba-closo-dodecaborane was prepared by a similar procedure. ¹H NMR (CDCl₃) δ 7.02 (d, 2H), 6.58 (d, 2H), 2.56 (t, 2H), 1.45-1.53 (m, 2H), 1.16-3.62 (m, 11H), 0.91 (t, 3H), MS (−ESI) calc. 309.448 (M−1), Obsv. 309.233.

### Synthesis of 1-Propylsulfinyl-12-(4-hydroxyphenyl)-1,12-dicarba-closo-dodecaborane

1-Propylsulfinyl-12-(4-hydroxyphenyl)-1,12-dicarba-closo-dodecaborane was prepared by a similar procedure. ¹H NMR (CDCl₃) δ 7.03 (d, 2H), 6.63 (d, 2H), 2.98 (t, 2H), 1.84-1.92 (m, 2H), 1.16-3.62 (m, 11H), 1.07 (t, 3H), MS (−ESI) calc. 325.447 (M−1), Obsv. 325.228.

### Synthesis of 1-Propylsulfonyl-12-(4-hydroxyphenyl)-1,12-dicarba-closo-dodecaborane

1-Propylsulfonyl-12-(4-hydroxyphenyl)-1,12-dicarba-closo-dodecaborane was prepared by a similar procedure. ¹H NMR (CDCl₃) δ 7.05 (d, 2H), 6.63 (d, 2H), 2.98 (t, 2H), 2.43-2.622 (m, 2H), 1.60-1.85 (m, 2H), 1.16-3.62 (m, 11H), 1.06 (t, 3H), MS (−ESI) calc. 341.2185 (M−1), Obsv. 341.2217.

### Synthesis of 1-Pentylthio-12-(4-hydroxyphenyl)-1,12-dicarba-closo-dodecaborane

1-Pentylthio-12-(4-hydroxyphenyl)-1,12-dicarba-closo-dodecaborane was prepared by a similar procedure. ¹H NMR (CDCl₃) δ 7.05 (d, 2H), 6.63 (d, 2H), 2.98 (t, 2H), 2.43-2.622 (m, 2H), 1.26-1.49 (m, 6H), 1.16-3.62 (m, 11H), 0.86 (t, 3H), MS (−ESI) calc. 337.502 (M−1), Obsv. 337.328.

### Synthesis of 1-Propylsulfinyl-12-(4-hydroxyphenyl)-1,12-dicarba-closo-dodecaborane

1-Propylsulfinyl-12-(4-hydroxyphenyl)-1,12-dicarba-closo-dodecaborane was prepared by a similar procedure. ¹H NMR (CDCl₃) δ 7.03 (d, 2H), 6.62 (d, 2H), 2.45-2.61 (m, 2H), 1.65-1.79 (m, 4H), 1.27-1.44 (m, 5H), 1.00-3.62 (m, 8H), 0.90 (t, 3H), MS (−ESI) calc. 353.2573 (M−1), Obsv. 353.2585.

### Synthesis of 1-Propylsulfonyl-12-(4-hydroxyphenyl)-1,12-dicarba-closo-dodecaborane

1-Propylsulfonyl-12-(4-hydroxyphenyl)-1,12-dicarba-closo-dodecaborane was prepared by a similar procedure. ¹H NMR (CDCl₃) δ 7.02 (d, 2H), 6.62 (d, 2H), 2.96-3.02 (m, 2H), 2.43-2.62 (m, 2H), 1.79-1.84 (m, 2H), 1.33-1.41 (m, 4H), 1.00-3.62 (m, 11H), 0.91 (t, 3H), MS (−ESI) calc. 369.2522 (M−1), Obsv. 369.2527.

### Synthesis of 1-Hexylthio-12-(4-hydroxyphenyl)-1,12-dicarba-closo-dodecaborane

1-Hexylthio-12-(4-hydroxyphenyl)-1,12-dicarba-closo-dodecaborane was prepared by a similar procedure. ¹H NMR (CDCl₃) δ 7.04 (d, 2H), 6.60 (d, 2H), 2.59 (t, 2H), 1.20-1.49 (m, 8H), 1.16-3.62 (m, 11H), 0.86 (t, 3H), MS (−ESI) calc. 351.529 (M−1), Obsv. 351.347.

### Synthesis of 1-Hexylsulfinyl-12-(4-hydroxyphenyl)-1,12-dicarba-closo-dodecaborane

1-Hexylsulfinyl-12-(4-hydroxyphenyl)-1,12-dicarba-closo-dodecaborane was prepared by a similar procedure. ¹H NMR (CDCl₃) δ 7.06 (d, 2H), 6.63 (d, 2H), 2.47-2.62 (m, 2H), 1.30-3.62 (m, 19H), 0.90 (t, 3H), MS (−ESI) calc. 368.2807 (M), Obsv. 367.2737 M−1).

### Synthesis of 1-Hexylsulfonyl-12-(4-hydroxyphenyl)-1,12-dicarba-closo-dodecaborane

1-Hexylsulfonyl-12-(4-hydroxyphenyl)-1,12-dicarba-closo-dodecaborane was prepared by a similar procedure. ¹H NMR (CDCl₃) δ 7.04 (d, 2H), 6.63 (d, 2H), 4.93 (bs, 1H), 2.97-3.01 (m, 2H), 1.30-3.63 (m, 18H), 0.91 (t, 3H), MS (−ESI) calc. 384.2756 (M), Obsv. 383.2687 (M−1).

### Synthesis of 1-(5-methyl-hexyl)thio-12-(4-hydroxyphenyl)-1,12-dicarba-closo-dodecaborane

1-(5-methyl-hexyl)thio-12-(4-hydroxyphenyl)-1,12-dicarba-closo-dodecaborane was prepared by a similar procedure. ¹H NMR (CDCl₃) δ 7.05 (d, 2H), 6.61 (d, 2H), 2.59 (t, 2H), 1.16-3.62 (m, 17H), 0.88 (d, 6H), MS (−ESI) calc. 333.3015 (M), Obsv. 365.2944 (M−1).

### Synthesis of 1-(5-methyl-hexyl)sulfinyl-12-(4-hydroxyphenyl)-1,12-dicarba-closo-dodecaborane

1-(5-methyl-hexyl)sulfinyl-12-(4-hydroxyphenyl)-1,12-dicarba-closo-dodecaborane was prepared by a similar procedure. ¹H NMR (CDCl₃) δ 7.06 (d, 2H), 6.63 (d, 2H), 4.94 (bs, 1H), 2.50-2.59 (m, 2H), 1.19-3.62 (m, 17H), 0.88 (d, 6H), MS (−ESI) calc. 382.2964 (M), Obsv. 381.2900 M−1).

### Synthesis of 1-(5-methyl-hexyl)sulfonyl-12-(4-hydroxyphenyl)-1,12-dicarba-closo-dodecaborane

1-(5-methyl-hexyl)sulfonyl-12-(4-hydroxyphenyl)-1,12-dicarba-closo-dodecaborane was prepared by a similar procedure. ¹H NMR (CDCl₃) δ 7.03 (d, 2H), 6.63 (d, 2H), 5.00 (bs, 1H), 2.97-3.02 (m, 2H), 1.16-3.63 (m, 17H), 0.88 (d, 6H), MS (−ESI) calc. 398.2913 (M), Obsv. 397.2851 (M−1).

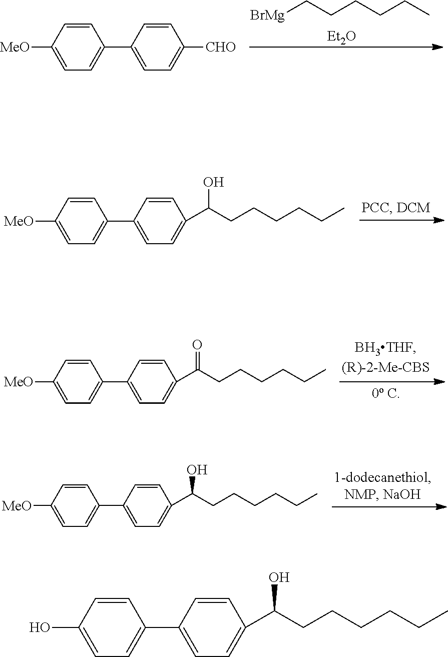

### Synthesis of 1-(4′-methoxy-[1,1′-biphenyl]-4-yl)heptan-1-ol

To a solution of 4′-methoxy-[1,1′-biphenyl]-4-carbaldehyde (0.69 g, 3.25 mmol) in anhydrous diethyl ether (25 ml) was added dropwise hexylmagnesium bromide (2 M in diethyl ether, 1.95 ml, 3.9 mmol) at 0° C. The reaction mixture stirred for another hour after addition and quenched by adding 0.1 N HCl (10 ml), the organic layer was separated, the aqueous layer was extracted with diethyl ether (2×20 ml). The combined organic layers were washed with water, NaHCO₃, and brine, dried over Na₂SO₄. Solvents were evaporated and the residue was purified by Teledyne Isco (RediSepRf column) to yield a yellow solid. 0.85 g pure product.

### Synthesis of 1-(4′-methoxy-[1,1′-biphenyl]-4-yl)heptan-1-one

Pyridinium chlorochromate (PCC, 0.9 g, 4.1 mmol) was suspended in anhydrous DCM (25 ml). A solution of 1-(4′-methoxy-[1,1′-biphenyl]-4-yl)heptan-1-ol (0.8 g, 2.68 mmol) in DCM (10 ml) was then added, and the reaction mixture was stirred at room temperature overnight. Diethyl ether (25 ml) was added followed by molecular sieves, and then stirred for 1 h. The supernatant was decanted and the insoluble residue was washed with dry ether (3×20 ml). The combined organic phases were passed through a short column of Celite followed by evaporation. The residue was purified by Teledyne Isco (RediSepRf column) to yield a white wax-like solid, pure product 0.64 g.

### Synthesis of (S)-1-(4′-methoxy-[1,1′-biphenyl]-4-yl)heptan-1-ol

Borane-tetrahydrofuran complex (10 mL, 10 mmol, 1.0 M solution in THF, stabilized with 0.005 M N-isopropyl-N-methyl-tert-butylamine (NEVIBA)) followed by (R)-2-methyl-CBS-oxazaborolidine [(2-MeCBS] (1.0 mL, 1.0 mmol, 1.0 M solution in toluene) were added to 10 mL anhydrous THF. The reaction mixture was stirred at room temperature for 15 minutes and 1-(4′-methoxy-[1,1′-biphenyl]-4-yl)heptan-1-one (0.29 g, 1.0 mmol) in 10 mL of anhydrous THF was added slowly over a period of 2 h at 0° C. The reaction mixture was stirred overnight at room temperature and then carefully quenched by addition of 2.0 M HCl (15 mL) in small portions to control H₂ development. Diethyl ether (15 mL) was added and the organic phase was washed brine and saturated NaHCO₃. The organic phase was dried over MgSO₄, filtered, and evaporated. The residue was purified by Teledyne Isco (RediSepRf column) to yield a white wax-like solid. Yield 0.21 g.

### Synthesis of (S)-4′-(1-hydroxyheptyl)-[1,1′-biphenyl]-4-ol

To a mixture of (S)-1-(4′-methoxy-[1,1′-biphenyl]-4-yl)heptan-1-ol (72 mg, 0.24 mmol), 1-dodecanethiol (75 mg, 89 μl, 0.37 mmol) in NMP (N-methylpyrrolidinone, 2 ml), NaOH (29 mg, 0.73 mmol) was added and the reaction mixture was heated up to 100° C. overnight. Cooled to room temperature, diluted with ethylacetate (15 ml), washed with 1N HCl (10 ml), water, and brine, and dried over Na₂SO₄. Solvents were evaporated and the residue was purified by Teledyne Isco (RediSepRf column) to yield a white solid, 42 mg pure product. ¹H NMR (CDCl₃) δ 7.48-7.55 (m, 4H), 7.42 (d, 2H) 6.93 (d, 2H), 4.74 (brs, 2H), 1.30-1.81 (m, 11H), 0.89 (t, 3H), HRMS calc. 283.17708 (M−1), obsv. 283.17184.

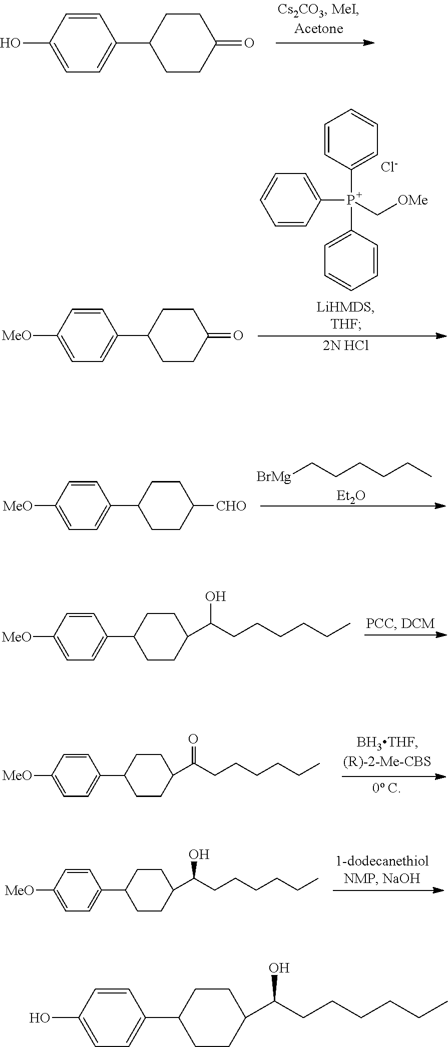

### Synthesis of 4-(4-methoxyphenyl)cyclohexan-1-one

The reaction mixture of 4-(4-hydroxyphenyl)cyclohexan-1-one (2.4 g, 12.62 mmol), Cs₂CO₃ (6.16 g, 18.91 mmol) and iodomethane (6 ml, 18.91 mmol) in acetone (50 ml) was heated to reflux for 3 h, cooled to room temperature, filtered, and washed with acetone (2×20 ml). The combined acetone filtrates were concentrated and the residue was purified by Teledyne Isco (RediSepRf column) to yield white solid, 2.58 g pure product.

### Synthesis of 4-(4-methoxyphenyl)cyclohexane-1-carbaldehyde

To a solution of (methoxymethyl) triphosphonium chloride (3.8 g, 11 mmol) in anhydrous THF 950 ml), lithium bis(trimehtylsilyl)amide (1.0 M in THF, 11 ml) was added dropwise at −78° C. The reaction mixture was stirred for 1 h, and a solution of 4-(4-methoxyphenyl)cyclohexan-1-one (2.04 g, 10 mmol) was added dropwise. This reaction mixture was stirred 30 min after addition, warmed up to room temperature, and stirred overnight. 2N HCl (50 ml) was added and stirred for 2 h. The reaction mixture was extracted with ethyl acetate (3×30 ml), the combined organic layers were washed with water, NaHCO₃and brine, and dried over Na₂SO₄. Solvents were evaporated and the residue was purified by Teledyne Isco (RediSepRf column) to yield a yellow solid. 1.25 g pure product.

### Synthesis of 1-(4-(4-methoxyphenyl)cyclohexyl)heptan-1-ol

To a solution of 4-(4-methoxyphenyl)cyclohexane-1-carbaldehyde (0.86 g, 3.94 mmol) in anhydrous diethyl ether (50 ml), hexylmagnesium bromide (2 M in diethyl ether, 2.46 ml, 4.52 mmol) was added dropwise at 0° C. The reaction mixture stirred for another hour after addition and quenched by adding 0.1 N HCl (20 ml), the organic layer was separated, the aqueous layer was extracted with diethyl ether (2×25 ml). The combined organic layers were washed with water, NaHCO₃, and brine, dried over Na₂SO₄. Solvents were evaporated and the residue was purified by Teledyne Isco (RediSepRf column) to yield a yellow solid. 0.99 g pure product.

### Synthesis of 1-(4-(4-methoxyphenyl)cyclohexyl)heptan-1-one

Pyridinium chlorochromate (PCC, 0.97 g, 4.42 mmol) was suspended in anhydrous DCM (25 ml). A solution of 1-(4-(4-methoxyphenyl)cyclohexyl)heptan-1-ol (0.88 g, 2.89 mmol) in DCM (10 ml) was then added, and the reaction mixture was stirred at room temperature overnight. Diethyl ether (25 ml) was added and followed by molecular sieves, and then stirred for 1 h. The supernatant was decanted and the insoluble residue was washed with dry ether (3×20 ml). The combined organic phases were passed through a short column of Celite followed by evaporation. The residue was purified by Teledyne Isco (RediSepRf column) to yield a white wax-like solid, pure product 0.72 g.

### Synthesis of (S)-1-(4-(4-methoxyphenyl)cyclohexyl)heptan-1-ol

Borane-tetrahydrofuran complex (21.5 mL, 21.5 mmol, 1.0 M solution in THF, stabilized with 0.005 M N-isopropyl-N-methyl-tert-butylamine (NEVIBA)) followed by (R)-2-methyl-CBS-oxazaborolidine [(2-MeCBS] (2.15 mL, 2.15 mmol, 1.0 M solution in toluene) were added to 20 mL anhydrous THF. The reaction mixture was stirred at room temperature for 15 minutes and 1-(4-(4-methoxyphenyl)cyclohexyl)heptan-1-one (0.65 g, 2.15 mmol) in 15 mL of anhydrous THF was added slowly over a period of 2 h at 0° C. The reaction mixture was stirred overnight at room temperature and then carefully quenched by addition of 2.0 M HCl (25 mL) in small portions to control H₂ development. Diethyl ether (25 mL) was added and the organic phase was washed brine and saturated NaHCO₃. The organic phase was dried over MgSO₄, filtered, and evaporated. The residue was purified by Teledyne Isco (RediSepRf column) to yield a white wax-like solid. Yield 0.50 g.

### Synthesis of (S)-4-(4-(1-hydroxyheptyl)cyclohexyl)phenol

To a mixture of (S)-1-(4-(4-methoxyphenyl)cyclohexyl)heptan-1-ol (0.25 g, 0.82 mmol), 1-dodecanethiol (0.26 g, 0.3 ml, 1.26 mmol) in NMP (N-methylpyrrolidinone, 5 ml), NaOH (100 mg, 2.48 mmol) was added and the reaction mixture was heated up to 100° C. overnight. Cooled to room temperature, diluted with ethyl acetate (15 ml), washed with 1N HCl (10 ml), water, and brine, and dried over Na₂SO₄. Solvents were evaporated and the residue was purified by Teledyne Isco (RediSepRf column) to yield white solid, 96 mg pure product.

¹H NMR (CDCl₃) δ 7.10 (d, 2H), 6.79 (d, 2H), 4.53 (s, 1H), 3.45 (m, 1H), 2.42 (m, 1H), 1.96 (m, 3H), 1.83 (m, 1H), 1.31-1.56 (m, 18H), 0.91 (t, 3H), HRMS calc. 289.21621 (M−1), obsv. 289.21902.

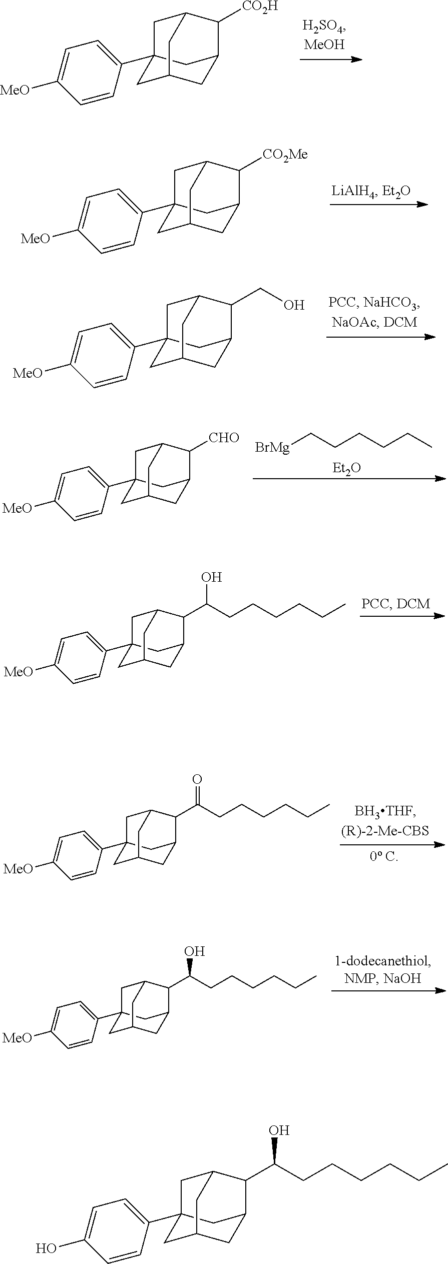

### Synthesis of methyl (1R,3S,5s,7s)-5-(4-methoxyphenyl)adamantane-2-carboxylate

The mixture of (1R,3S,5s,7s)-5-(4-methoxyphenyl)adamantane-2-carboxylic acid (0.59 g, 2.06 mmol), conc. H₂SO₄ (1 ml) in methanol (50 ml) was heated up to reflux overnight. Cooled down to room temperature, methanol was evaporated and the residue was neutralized with saturated sodium bicarbonate solution, and extracted with ethyl acetate (3×2 ml). The combined organic layers were washed with water and brine, dried over Na₂SO₄. Solvents were evaporated to yield an off-white solid. 0.62 g crude product. Used directly in next reaction without further purification.

### Synthesis of ((1R,3S,5s,7s)-5-(4-methoxyphenyl)adamantan-2-yl)methanol

The methyl (1R,3S,5s,7s)-5-(4-methoxyphenyl)adamantane-2-carboxylate (crude product from last reaction 0.62 g, 2.06 mmol) was dissolved in anhydrous diethyl ether (50 ml), and treated with LAH (160 mg, 4.21 mmol) at 0° C. for 2 h. 2N NaOH was added dropwise until the formation of a white precipitate, filtered, and washed with diethyl ether (3×30 ml). The combined organic layers were dried over Na₂SO₄. Solvents were evaporated and the residue was purified by Teledyne Isco (RediSepRf column) to yield a white solid, 498 mg pure product.

### Synthesis of (1R,3S,5s,7s)-5-(4-methoxyphenyl)adamantane-2-carbaldehyde

To a mixture of ((1R,3S,5s,7s)-5-(4-methoxyphenyl)adamantan-2-yl)methanol (0.46 g, 1.7 mmol), NaHCO₃ (0.14 g, 1.7 mmol), NaOAc (143 mg, 1.7 mmol) in anhydrous DCM, pyridinium chlorochromate (PCC, 0.37 g, 1.7 mmol) was added. The reaction mixture was stirred at room temperature for 3 h. Filtered, and the filtrate was washed with 1N HCl, water, NaHCO₃, and brine, and dried over Na₂SO₄. Solvents were evaporated and the residue was purified by Teledyne Isco (RediSepRf column) to yield a white solid, 290 mg pure product.

### Synthesis of 1-((1R,3S,5s,7s)-5-(4-methoxyphenyl)adamantan-2-yl)heptan-1-ol

To a solution of (1R,3S,5s,7s)-5-(4-methoxyphenyl)adamantane-2-carbaldehyde (0.26 g, 0.96 mmol) in anhydrous diethyl ether (20 ml), hexylmagnesium bromide (2 M in diethyl ether, 0.6 ml, 1.2 mmol) was added dropwise at 0° C. The reaction mixture stirred for another hour after addition and quenched by adding 0.1 N HCl (10 ml), the organic layer was separated, the aqueous layer was extracted with diethyl ether (2×20 ml). The combined organic layers were washed with water, NaHCO₃, and brine, dried over Na₂SO₄. Solvents were evaporated and the residue was purified by Teledyne Isco (RediSepRf column) to yield a yellow solid. 287 mg pure product.

### Synthesis of 1-((1R,3S,5s,7s)-5-(4-methoxyphenyl)adamantan-2-yl)heptan-1-one

Pyridinium chlorochromate (PCC, 0.25 g, 1.16 mmol) was suspended in anhydrous DCM (25 ml). A solution of 1-((1R,3S,5s,7s)-5-(4-methoxyphenyl)adamantan-2-yl)heptan-1-ol (0.27 g, 0.76 mmol) in DCM (10 ml) was then added, and the reaction mixture was stirred at room temperature overnight. Diethyl ether (25 ml) was added and followed by molecular sieves, and then stirred for 1 h. The supernatant was decanted and the insoluble residue was washed with dry ether (3×20 ml). The combined organic phases were passed through a short column of Celite followed by evaporation. The residue was purified by Teledyne Isco (RediSepRf column) to yield a white wax-like solid, pure product 235 mg.

### Synthesis of (1S)-1-((1R,3S,5R,7R)-5-(4-methoxyphenyl)adamantan-2-yl)heptan-1-ol

Borane-tetrahydrofuran complex (5.9 mL, 21.5 mmol, 1.0 M solution in THF, stabilized with 0.005 M N-isopropyl-N-methyl-tert-butylamine (NEVIBA)) followed by (R)-2-methyl-CBS-oxazaborolidine [(2-MeCBS] (0.59 mL, 0.59 mmol, 1.0 M solution in toluene) were added to 20 mL anhydrous THF. The reaction mixture was stirred at room temperature for 15 minutes and 1-((1R,3S,5s,7s)-5-(4-methoxyphenyl)adamantan-2-yl)heptan-1-one (0.21 g, 0.59 mmol) in 10 mL of anhydrous THF was added slowly over a period of 2 h at 0° C. The reaction mixture was stirred overnight at room temperature and then carefully quenched by addition of 2.0 M HCl (25 mL) in small portions to control H₂ development. Diethyl ether (25 mL) was added and the organic phase was washed brine and saturated NaHCO₃. The organic phase was dried over MgSO₄, filtered, and evaporated. The residue was purified by Teledyne Isco (RediSepRf column) to yield a white wax-like solid. Yield 158 mg.

### Synthesis of 4-((1R,3R,5S,7R)-4-((S)-1-hydroxyheptyl)adamantan-1-yl)phenol

To a mixture of (1S)-1-((1R,3S,5R,7R)-5-(4-methoxyphenyl)adamantan-2-yl)heptan-1-ol (132 mg, 0.37 mmol), 1-dodecanethiol (0.21 g, 0.24 ml, 0.56 mmol) in NMP (N-methylpyrrolidinone, 5 ml), NaOH (67.2 mg, 1.68 mmol) was added and the reaction mixture was degassed with Ar, then heated up to 130° C. overnight. Cooled to room temperature, diluted with ethyl acetate (15 ml), washed with 1N HCl (10 ml), water, and brine, and dried over Na₂SO₄. Solvents were evaporated and the residue was purified by Teledyne Isco (RediSepRf column) to yield white solid, 62 mg pure product. ¹H NMR (CDCl₃) δ 7.27 (d, 2H), 6.81 (d, 2H), 4.56 (s, 1H), 3.12 (brs, 1H), 2.22 (brs, 2H), 1.55-1.85 (m, 24H), 0.90 (t, 3H), HRMS calc. 341.2319 (M−1), obsv. 315.2372.

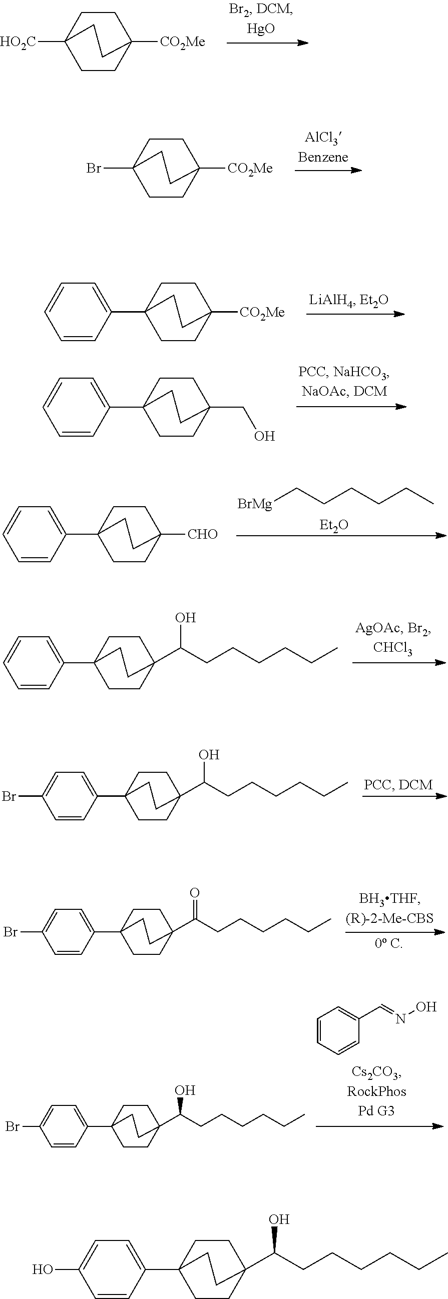

### Synthesis of methyl 4-bromobicyclo[2.2.2]octane-1-carboxylate

A solution of bromine (3.3 g, 20.6 mmol) in dichloromethane (20 ml) was added dropwise over 10 min into a heterogeneous refluxing mixture of 4-(methoxycarbonyl)bicyclo[2.2.2]octane-1-carboxylic acid (3.0 g, 13.90 mmol) and mercuric oxide (5.12 g) in dichloromethane (60 ml), and heating was continued for 3.5 h. After the reaction mixture was allowed to cool to room temperature, it was filtered and the resulting light orange filtrate was treated with MgSO₄ and filtered again. The volatiles were removed and the residue was purified by Teledyne Isco (RediSepRf column) to yield a white wax-like solid, pure product 1.93 g.

### Synthesis of methyl 4-phenylbicyclo[2.2.2]octane-1-carboxylate

A benzene (30 ml) solution of methyl 4-bromobicyclo[2.2.2]octane-1-carboxylate (1.90 g, 7.7 mmol) was added dropwise to a cooled (˜−12° C.) mixture of benzene (100 ml) and aluminum chloride (5.0 g, 35 mmol) over 15 min. The heterogeneous mixture was stirred for 1 h while allowing the cooling bath to warm gradually to 3° C. and then stirred at room temperature overnight. Diluted with diethyl ether (100 ml), washed with 1N HCl, water, and brine, and dried over Na₂SO₄. Solvents were evaporated and the residue was purified by Teledyne Isco (RediSepRf column) to yield a white solid, 1.6 g pure product.

### Synthesis of (4-phenylbicyclo[2.2.2]octan-1-yl)methanol

The methyl 4-phenylbicyclo[2.2.2]octane-1-carboxylate (0.51 g, 2.1 mmol) was dissolved in anhydrous diethyl ether (25 ml), treated with LAH (159 mg, 4.2 mmol) at 0° C. for 2 h. 2N NaOH was added dropwise until form white precipitation, filtered, washed with diethyl ether (3×30 ml). The combine organic layers were dried over Na₂SO₄. Solvents were evaporated and the residue was purified by Teledyne Isco (RediSepRf column) to yield a white solid, 0.45 g pure product.

### Synthesis of 4-phenylbicyclo[2.2.2]octane-1-carbaldehyde

To a mixture of (4-phenylbicyclo[2.2.2]octan-1-yl)methanol (0.43 g, 1.99 mmol), NaHCO₃ (166 mg, 1.99 mmol), NaOAc (163 mg, 1.99 mmol) in anhydrous DCM, pyridinium chlorochromate (PCC, 0.43 g, 1.99 mmol) was added. The reaction mixture was stirred at room temperature for 3 h. Filtered, and the filtrate was washed with 1N HCl, water, NaHCO₃, and brine, dried over Na₂SO₄. Solvents were evaporated and the residue was purified by Teledyne Isco (RediSepRf column) to yield a white solid, 405 mg pure product.

### Synthesis of 1-(4-phenylbicyclo[2.2.2]octan-1-yl)heptan-1-ol

To a solution 4-phenylbicyclo[2.2.2]octane-1-carbaldehyde (0.4 g, 1.87 mmol) in anhydrous diethyl ether (25 ml), hexylmagnesium bromide (2 M in diethyl ether, 02.0 ml, 4.0 mmol) was added dropwise at 0° C. The reaction mixture stirred for another hour after addition and quenched by adding 0.1 N HCl (10 ml), the organic layer was separated, the aqueous layer was extracted with diethyl ether (2×20 ml). The combined organic layers were washed with water, NaHCO₃, and brine, dried over Na₂SO₄. Solvents were evaporated and the residue was purified by Teledyne Isco (RediSepRf column) to yield a yellow solid. 0.48 g pure product.

### Synthesis of 1-(4-(4-bromophenyl)bicyclo[2.2.2]octan-1-yl)heptan-1-ol

To a mixture of 1-(4-phenylbicyclo[2.2.2]octan-1-yl)heptan-1-ol (0.28, 0.94 mmol), silver acetate (0.24, 1.09 mmol) in chloroform (25 ml), a solution of bromine (0.16 g, 0.99 mmol) in chloroform (10 ml) was added dropwise at 0° and stirred for 3 h and then warmed up to room temperature. Washed with NaHCO₃, water and brine, dried over Na₂SO₄. Solvents were evaporated and the residue was purified by Teledyne Isco (RediSepRf column) to yield a yellow solid. 0.28 g pure product.

### Synthesis of 1-(4-(4-bromophenyl)bicyclo[2.2.2]octan-1-yl)heptan-1-one

Pyridinium chlorochromate (PCC, 0.27 g, 1.27 mmol) was suspended in anhydrous DCM (25 ml). A solution of 1-(4-(4-bromophenyl)bicyclo[2.2.2]octan-1-yl)heptan-1-ol (0.16 g, 0.42 mmol) in DCM (10 ml) was then added, and the reaction mixture was stirred at room temperature overnight. Diethyl ether (25 ml) was added and followed by molecular sieves, and then stirred for 1 h. The supernatant was decanted and the insoluble residue was washed with dry ether (3×20 ml). The combined organic phases were passed through a short column of Celite followed by evaporation. The residue was purified by Teledyne Isco (RediSepRf column) to yield a white wax-like solid, pure product 135 mg.

### Synthesis of (S)-1-(4-(4-bromophenyl)bicyclo[2.2.2]octan-1-yl)heptan-1-ol

Borane-tetrahydrofuran complex (3.2 ml, 3.2 mmol, 1.0 M solution in THF, stabilized with 0.005 M N-isopropyl-N-methyl-tert-butylamine (NEVIBA)) followed by (R)-2-methyl-CBS-oxazaborolidine [(2-MeCBS] (0.32 mL, 0.32 mmol, 1.0 M solution in toluene) were added to 20 mL anhydrous THF. The reaction mixture was stirred at room temperature for 15 minutes and 1-(4-(4-bromophenyl)bicyclo[2.2.2]octan-1-yl)heptan-1-one (0.12 g, 0.32 mmol) in 10 mL of anhydrous THF was added slowly over a period of 2 h at 0° C. The reaction mixture was stirred overnight at room temperature and then carefully quenched by addition of 2.0 M HCl (25 mL) in small portions to control H₂ development. Diethyl ether (25 mL) was added and the organic phase was washed brine and saturated NaHCO₃. The organic phase was dried over MgSO₄, filtered, and evaporated. The residue was purified by Teledyne Isco (RediSepRf column) to yield a white wax-like solid. Yield 98 mg.

### Synthesis of (S)-4-(4-(1-hydroxyheptyl)bicyclo[2.2.2]octan-1-yl)phenol

A mixture of (S)-1-(4-(4-bromophenyl)bicyclo[2.2.2]octan-1-yl)heptan-1-ol (72 mg, 0.19 mmol), benzaldehyde oxime (30 mg, 0.25 mmol), Cs₂CO₃ (136.2 mg, 0.42 mmol), and RockPhos Pd G3 (8 mg) in DMF (1 ml) was degassed with Ar for 15 min. Then, the mixture was heated to 80° C. for 18 h. The mixture was then cooled down room temperature, diluted with ethyl acetate (10 ml), washed with 1N HCl (10 ml), water, and brine, dried over Na₂SO₄, filtered, and evaporated. The residue was purified by Teledyne Isco (RediSepRf column) to yield a white solid, 98 mg. ¹H NMR (CDCl₃) δ 7.21 (d, 2H), 6.79 (d, 2H), 3.22 (d, 1H), 1.81 (t, 6H), 1.28-1.61 (m, 18H), 0.90 (t, 3H), HRMS calc. 315.23186 (M−1), obsv. 315.23676.

The selectivity and potency of various example compounds in the second library was carried out via in vitro testing in ERα and ERβ cell-based reporter assays. The results are included in Table 8 below.

TABLE 8
Results of in vitro testing of the compounds in ERα
and ERβ cell-based reporter assays.

| | EC₅₀ (nM) | | |
|---|---|---|---|
| Compound | ERα agonism | ERβ agonism | Selectivity |
| 26 | >10,000 | >10,000 | — |
| 27 | >10,000 | >10,000 | — |
| 28 | >10,000 | 4,125 | — |
| 29 | >10,000 | >10,000 | — |
| 30 | >10,000 | >10,000 | — |
| 31 | >10,000 | 4,452 | — |
| 32 | 92.3 | 2.2 | 41.95 |
| 33 | 1,800 | 72.5 | 24.83 |
| 34 | 318.5 | 20.9 | 15.24 |
| 35 | 59.02 | 3.9 | 15.13 |
| 36 | 634.8 | 73.2 | 8.67 |
| 37 | 4,575 | 103 | 44.42 |
| 38 | 109.3 | 5.3 | 20.62 |
| 39 | 128.6 | 10.7 | 12.02 |
| 40 | 1,297 | 118.8 | 10.92 |
| 41 | 60.6 | 2.7 | 22.44 |
| 42 | 265.8 | 17 | 15.64 |
| 43 | 153.6 | 12.4 | 12.39 |
| 44 | 1,490 | 17.9 | 83.24 |
| 45 | 62.2 | 2.4 | 25.92 |
| 46 | 327.9 | 30 | 10.93 |

### Example 23. Evaluation of Example Carborane for the Treatment of Fibrotic Conditions

The in vivo efficacy of compound 25 (shown below) was evaluated in a STAM model of non-alcoholic steatohepatitis (NASH, a fibrotic condition).

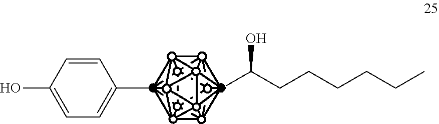

Materials and Methods

Compound 25 was prepared as described above. To prepare dosing solutions, compound was weighed and suspended in vehicle (5% DMSO, 5% Tween® 20, water). Compound 25 was administered orally in a volume of 10 mL/kg. Compound 25 was administered at two dose levels of 10 and 100 mg/kg once daily.

Pathogen-free 14 day-pregnant C₅₇BU6 mice were obtained for use in this study. All animals used in this study were housed and cared for in accordance with industry standards. NASH was established in mule mice by a single subcutaneous injection of 200 μg streptozotocin (STZ, Sigma Aldrich, USA) two days after birth and feeding with a high fat diet (HFD, 57 kcal % fat, Cat #HFD32, CLEA Japan Inc., Japan) ad libitum after 4 weeks of age (day 28). NASH mice were randomized into three groups of eight mice at five weeks of age (day 35 f 2) the day before the start of treatment based on their body weight. Littermate control mice without STZ priming (n=8) were set up for control purposes. Individual body weight was measured daily during the treatment period. Survival, clinical signs, and behavior of the mice was also monitored daily.

Measurement of Plasma Biochemistry. To evaluate plasma biochemistry, non-fasting blood was collected in polypropylene tubes with anticoagulant (Novo-Heparin, Mochida Pharmaceutical Co. Ltd., Japan) and centrifuged at 1,000×g for 15 minutes at 4° C. The supernatant was collected and stored at −80° C. until use. Plasma ALT levels were measured by FUJI DRI-CHEM 7000 (Fujifilm, Japan).

Measurement of liver biochemistry. Liver total lipid-extracts were obtained by Folch's method (Folch J. et al., J. Biol. Chem. 1957; 226: 497). Liver samples were homogenized in chloroform-methanol (2:1, v/v) and incubated overnight at room temperature. After washing with chloroform-methanol-water (8:4:3, v/v/v), the extracts were evaporated to dryness, and dissolved in isopropanol. Liver triglyceride contents were measured by Triglyceride E-test (Wako Pure Chemical Industries, Ltd., Japan).

Histological Analysis. For HE staining, sections were cut from paraffin blocks of liver tissue prefixed in Bouin's solution and stained with Lillie-Mayer's Hematoxylin (Muto Pure Chemicals Co., Ltd., Japan) and eosin solution (Wako Pure Chemical Industries). NAFLD Activity score (NAS) was calculated according to the criteria of Kleiner (Kleiner D E. et al., Hepatology, 2005; 41:1313). To visualize collagen deposition, Bouin's fixed liver sections were stained using picro-Sirius red solution (Waldeck, Germany). For quantitative analysis of fibrosis area, bright field images of Sirius red-stained sections were captured around the central vein using a digital camera (DFC295; Leica, Germany) at 200-fold magnification, and the positive areas in 5 fields/section were measured using ImageJ software (National Institute of Health, USA).

Sample collection. For plasma samples, non-fasting blood was collected in polypropylene tubes with anticoagulant (Novo-Heparin) and centrifuged at 1,000×g for 15 minutes at 4° C. The supernatant was collected and stored at −80° C. for biochemistry (20 μL) and shipping (remaining).

For liver samples, left lateral lobe was collected and cut into six pieces. Two pieces of left lateral lobe, left and right medial lobes, and caudate lobe were snap frozen in liquid nitrogen and stored at −80° C. for shipping. The other two pieces of left lateral lobe were fixed in Bouin's solution and then embedded in paraffin. Paraffin blocks were stored at room temperature for histology. The remaining pieces of left lateral lobe were embedded in O.C.T. compound and quick frozen in liquid nitrogen. O.C.T. blocks were stored at −80° C. The right lobe was snap frozen in liquid nitrogen and stored at −80° C. for liver biochemistry.

Statistical tests. Statistical analyses were performed using Bonferroni Multiple Comparison Test on GraphPad Prism 6 (GraphPad Software Inc., USA). P values <0.05 were considered statistically significant. A trend or tendency was assumed when a one-tailed t-test returned P values <0.1. Results were expressed as mean f SD.

Experimental Design and Treatment

Study Groups. The populations of mice were divided into four study groups:

Group 1: Normal. Eight normal mice were kept without any treatment until sacrifice.

Group 2: Vehicle. Eight NASH mice were orally administered vehicle (5% DMSO, 5% Tween® 20, water) in a volume of 10 mL./kg once daily from 5 to 12 weeks of age.

Group 3: Compound High. Eight NASH mice were orally administered vehicle supplemented with compound 25 at a dose of 100 mL/kg once daily from S to 12 weeks of age.

Group 4: Compound Low. Eight NASH mice were orally administered vehicle supplemented with compound 25 at a dose of 10 mL/kg once daily from 5 to 12 weeks of age.

The table below summarizes the treatment schedule.

| | No. | | | Dose | Volume | | Sacrifice |
|---|---|---|---|---|---|---|---|
| Group | mice | Mice | Test substance | (mg/kg) | (mL/kg) | Regimen | (wks) |
| 1 | 8 | Normal | — | — | — | — | 12 |
| 2 | 8 | STAM | Vehicle | — | 10 | PO, QD, | 12 |
| | | | | | | 5-12 wks | |
| 3 | 8 | STAM | Compound 25 | 100 | 10 | PO, QD, | 12 |
| | | | | | | 5-12 wks | |
| 4 | 8 | STAM | Compound 25 | 10 | 10 | PO, QD, | 12 |
| | | | | | | 5-12 wks | |

PO = orally;
QD = once daily

Animal Monitoring and Sacrifice. The viability, clinical signs and behavior were monitored daily. Body weight was recorded before the treatment. Mice were observed for significant clinical signs of toxicity, moribundity and mortality approximately 60 minutes after each administration. The animals were sacrificed at 12 weeks of age by exsanguination through direct cardiac puncture under isoflurane anesthesia (Pfizer Inc.).

Results

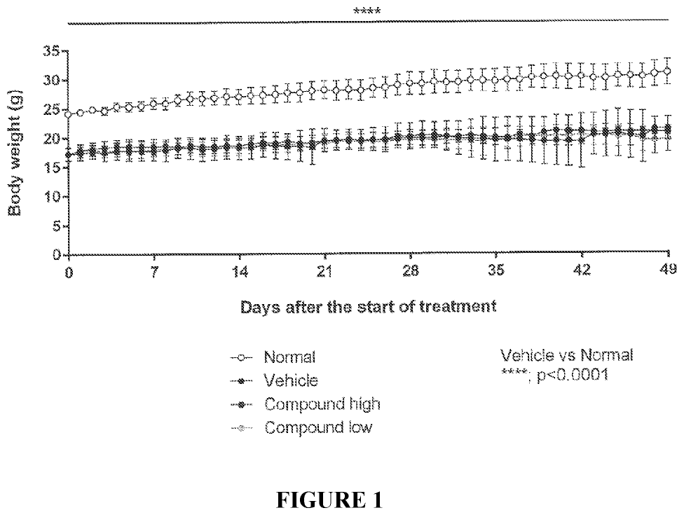

Body weight changes and general condition. FIG. 1 illustrates the average body weight change observed in the four study groups over the course of the treatment period. Mean body weight in all groups gradually increased during the treatment period. Mean body weights of the Vehicle group were significantly lower than that of the Normal group from Day 0 to Day 49. There were no significant differences in mean body weights at any day during the treatment period between the Vehicle group and the Compound treatment groups.

During the treatment period, mice found dead before reaching Day 49 were as follows; three out of 8 mice were found dead in the Vehicle group. Two out of 8 mice were found dead in the Compound high and Compound low groups.

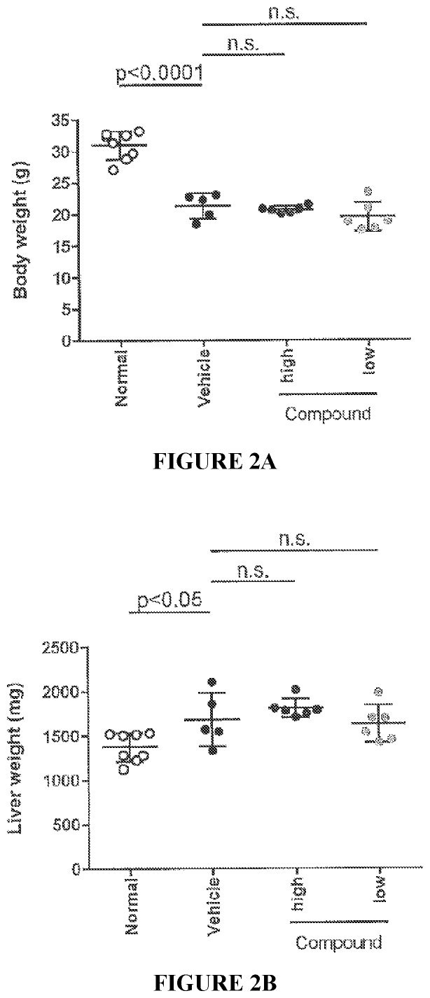

Body weight on the day of sacrifice and liver weight. FIG. 2A is a plot showing the body weight of animals on the day of sacrifice. The Vehicle group showed a significant decrease in mean body weight on the day of sacrifice compared with the Normal group. There were no significant differences in mean body weight on the day of sacrifice between the Vehicle group and the Compound treatment groups.

FIG. 2B is a plot showing the liver weight of animals on the day of sacrifice. The Vehicle group showed a significant increase in mean liver weight compared with the Normal group. There were no significant differences in mean liver weight between the Vehicle group and the Compound treatment groups

FIG. 2C is a plot showing the liver-to-body weight ratio of animals on the day of sacrifice. The Vehicle group showed a significant increase in mean liver-to-body weight ratio compared with the Normal group. Mean liver-to-body weight ratio in the Compound high group tended to increase compared with the Vehicle group. There was no significant difference in mean liver-to-body weight ratio between the Vehicle group and the Compound low group

The results of these studies are summarized in the table below.

| | | | Compound | Compound |
|---|---|---|---|---|
| Parameter | Normal | Vehicle | High | Low |
| (mean ± SD) | (n = 8) | (n = 5) | (n = 6) | (n = 6) |
| Body Weight | 31.0 ± 2.2 | 21.3 ± 2.0 | 20.8 ± 0.5 | 19.6 ± 2.3 |
| (g) | | | | |
| Liver Weight | 1375 ± 163 | 1683 ± 302 | 1809 ± 106 | 1631 ± 208 |
| (mg) | | | | |
| Liver-to-Body | 4.4 ± 0.4 | 7.9 ± 1.2 | 8.7 ± 0.5 | 8.4 ± 1.7 |
| Weight Ratio | | | | |
| (%) | | | | |

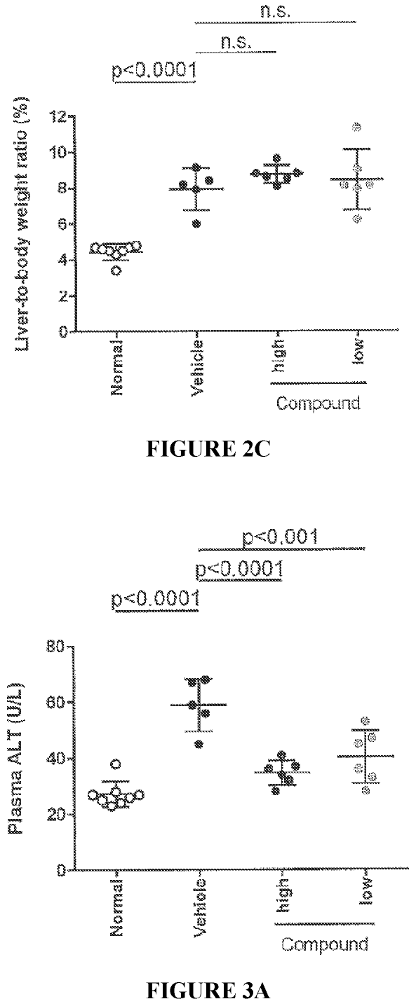

Biochemistry. FIG. 3A is a plot showing the plasma alanine aminotransferase (ALT) levels on the day of sacrifice. The Vehicle group showed a significant increase in plasma ALT level compared with the Normal group. The Compound high and low groups showed significant decreases in plasma ALT levels compared with the Vehicle group

FIG. 3B is a plot showing liver triglyceride levels (in mg/g liver) on the day of sacrifice. The Vehicle group showed a significant increase in liver triglyceride content compared with the Normal group. The Compound high and low groups showed significant decreases in liver triglyceride compared with the Vehicle group.

The results of these studies are summarized in the table below.

| | | | Compound | Compound |
|---|---|---|---|---|
| Parameter | Normal | Vehicle | High | Low |
| (mean ± SD) | (n = 8) | (n = 5) | (n = 6) | (n = 6) |
| Plasma ALT | 27 ± 5 | 59 ± 9 | 35 ± 4 | 40 ± 10 |
| (U/L) | | | | |
| Liver Triglycerides | 5.2 ± 1.4 | 59.7 ± 9.9 | 18.2 ± 8.0 | 34.0 ± 9.3 |
| (mg/g liver) | | | | |

Histological analyses. Liver sections were HE-stained and imaged as described above. Steatosis, lobular inflammation, and hepatocyte ballooning was evaluated to calculate a NAFLD Activity Score. The definition of NAS components is included in the table below.

| Item | Score | Extent |
|---|---|---|
| Steatosis | 0 | <5% |
| | 1 | 5-33% |
| | 2 | >33-66% |
| | 3 | >66% |
| Lobular | 0 | No foci |
| Inflammation | 1 | <2 foci/200x |
| | 2 | 2-4 foci/200x |
| | 3 | >4 foci/200x |
| Hepatocyte | 0 | None |
| Ballooning | 1 | Few balloon cells |
| | 2 | Many cells/prominent ballooning |

Liver sections from the Vehicle group exhibited micro- and macrovesicular fat deposition, hepatocellular ballooning and inflammatory cell infiltration compared with the Normal group. The Vehicle group showed a significant increase in NAS compared with the Normal group. NAS in the Compound high and low groups tended to decrease compared with the Vehicle group.

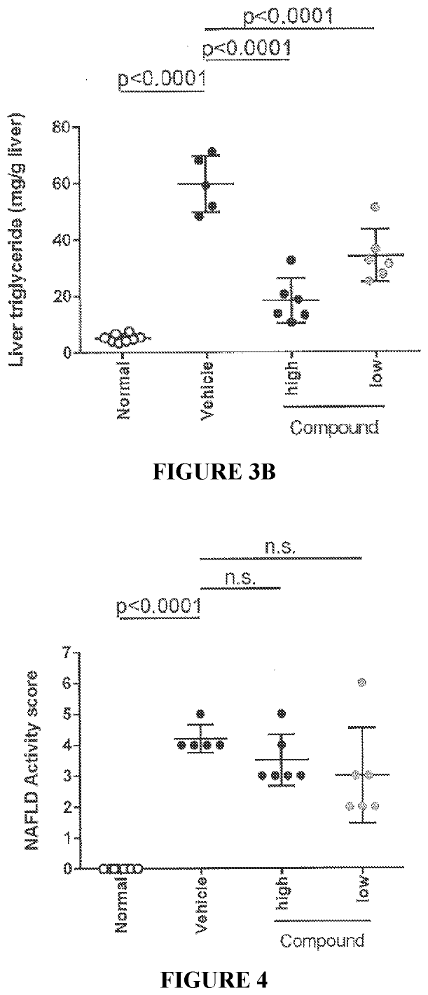

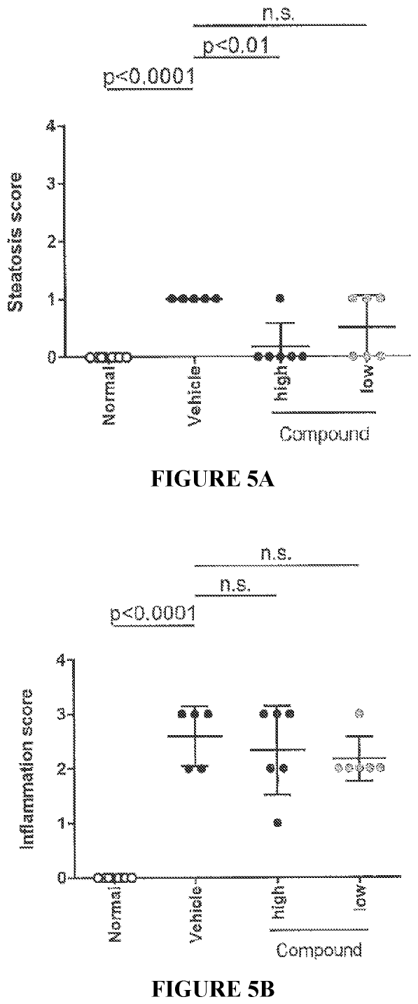

FIG. 4 is a plot showing the non-alcoholic fatty liver disease (NAFLD) activity score on the day of sacrifice. FIG. 5A is a plot showing the steatosis score on the day of sacrifice. FIG. 5B is a plot showing the inflammation score on the day of sacrifice. FIG. 5C is a plot showing the ballooning score on the day of sacrifice. The results of these studies are summarized in the table below.

| | | Score | | | | | | | | | | |
|---|---|---|---|---|---|---|---|---|---|---|---|---|
| | | | | | | Lobular | | | | Hepatocyte | | |
| | | Steatosis | | | | Inflammation | | | | Ballooning | | |
| Group | n | 0 | 1 | 2 | 3 | 0 | 1 | 2 | 3 | 0 | 1 | 2 |
| Normal | 8 | 8 | — | — | — | 8 | — | — | — | 8 | — | — |
| Vehicle | 5 | — | 5 | — | — | — | — | 2 | 3 | 2 | 3 | — |
| Compound | 6 | 5 | 1 | — | — | — | 1 | 2 | 3 | 2 | 2 | 2 |
| high | | | | | | | | | | | | |
| Compound | 6 | 3 | 3 | — | — | — | — | 5 | 1 | 5 | — | 1 |
| low | | | | | | | | | | | | |

Sirius red staining and the fibrosis area. Liver sections were stained with Sirius Red an imaged, and the positive area was determined as described above. Liver sections from the Vehicle group showed increased collagen deposition in the pericentral region of liver lobule compared with the Normal group. The Vehicle group showed a significant increase in the fibrosis area (Sirius red-positive area) compared with the Normal group. The Compound high group showed a significant decrease in the fibrosis area compared with the Vehicle group.

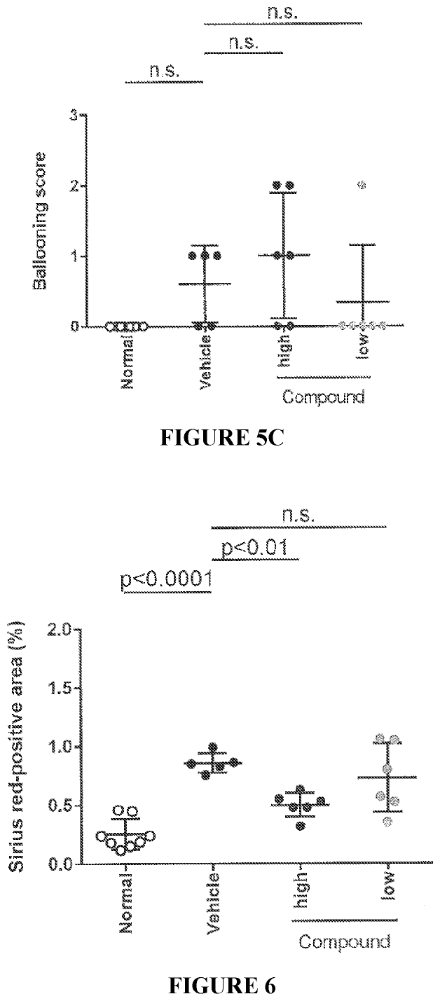

FIG. 6 is a plot showing the fibrosis area (sirius red-positive area, %) on the day of sacrifice. The results of these studies are summarized in the table below.

| | | | Compound | Compound |
|---|---|---|---|---|
| Parameter | Normal | Vehicle | High | Low |
| (mean ± SD) | (n = 8) | (n = 5) | (n = 6) | (n = 6) |
| Sirius | 0.25 ± 0.13 | 0.86 ± 0.08 | 0.50 ± 0.10 | 0.73 ± 0.29 |
| red-positive | | | | |
| area (%) | | | | |

Summary and Conclusion

Treatment with compound 25 showed significant reduction in plasma ALT levels and liver triglycelide content compared with Vehicle group. Treatment with compound 25 showed a decreasing trend in NAFLD Activity Score (NAS) compared with Vehicle group. Treatment with compound 25 of high dose showed significant reduction in the fibrosis area compared with Vehicle group, in a dose dependent manner.

In conclusion, the compound 25 showed hepatoprotective potential, anti-steatosis and anti-fibrosis effects in this NASH model

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for reducing fibrosis in a cell or tissue comprising contacting the cell or tissue with a carborane or carborane analog in an effective amount to decrease or inhibit the fibrosis, wherein the carborane or carborane analog comprises an ERβ agonist.

2. A method of treating a fibrotic condition comprising administering a carborane or carborane analog to a subject in need thereof, in an effective amount to decrease or inhibit the fibrotic condition in the subject, wherein the carborane or carborane analog comprises an ERβ agonist.

3. The method of claim 1, wherein the carborane or carborane analog comprises a compound defined by Formula I, or a pharmaceutically acceptable salt thereof 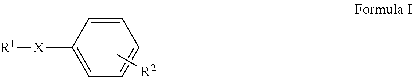
wherein
R¹ represents a dicarba-closo-dodecaboran-yl group which may have one or more substituents selected from the group consisting of an alkyl group, an alkenyl group, a carboxyl group, an alkoxycarbonyl group, an amino group, a hydroxyl group, a hydroxyalkyl group, a mono or di-alkylcarbamoyl-substituted alkyl group, an alkanoyl group, an aryl group, and an aralkyl group, each of which may be substituted or unsubstituted;
R² represents a carboxyl group, an alkoxycarbonyl group, or a hydroxyl group; and
X represents a single bond, or a linking group selected from the group consisting of groups represented by the following formulas: 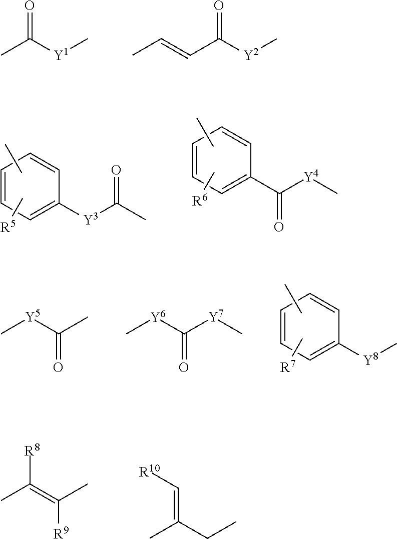
wherein Y¹, Y², Y³, Y⁴ Y⁵, Y⁶, and Y⁷ independently represent an oxygen atom or —N(R³)— wherein R³ represents hydrogen atom or an alkyl group; Y⁸represents an oxygen atom, —N(R⁴)— wherein R⁴ represents hydrogen atom or an alkyl group, —CO—, —CH₂—, or —C(═CH²)—; R⁵, R⁶, and R⁷ independently represent hydrogen or one or more substituents on the phenyl group; R⁸ represents an alkyl group or an aryl group which may be substituted; R⁹represents an alkyl group; and R¹⁰represents a substituted or unsubstituted aryl group.

4. The method of claim 1, wherein the carborane or carborane analog comprises a compound defined by Formula II, or a pharmaceutically acceptable salt thereof 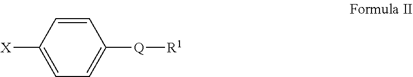
wherein
Q is a substituted or unsubstituted dicarba-closo-dodecaborane cluster, and 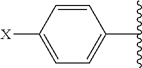
and R¹ are attached to Q in a para configuration;
X is OH, NHR², SH, or S(O)(O)NHR²;
R¹ is substituted or unsubstituted C₄-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteraryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, or NR³R⁴;
R² is H, OH, halogen, or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl.

5-8. (canceled)

9. The method of claim 1, wherein the carborane or carborane analog comprises a compound defined by Formula XL, or a pharmaceutically acceptable salt thereof 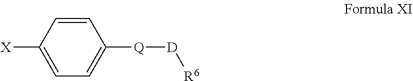
wherein
Q is a substituted or unsubstituted dicarba-closo-dodecaborane cluster;
D is —S—, —S(O)—, —S(O)(O)—, —S(O)(NH)—, —P(O)(OH)O—, —P(O)(OH)NH—, or —O—;
X is OH, NHR², SH, or S(O)(O)NHR²;
R⁶ is substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₂-C₂₀ alkylheteraryl, substituted or unsubstituted C₄-C₂₀ alkylcycloalkyl, or substituted or unsubstituted C₄-C₂₀ alkylheterocycloalkyl; and
R² is H, OH, halogen, or substituted or unsubstituted C₁-C₄ alkyl.

10. The method of claim 1, wherein the carborane or carborane analog comprises a compound defined by Formula XII, or a pharmaceutically acceptable salt thereof
A-Q-R¹ Formula XII
wherein
Q is a substituted or unsubstituted dicarba-closo-dodecaborane cluster, and A and R¹ are attached to Q in a para configuration;
A is a substituted or unsubstituted heteroaryl ring;
R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)N R³R⁴, —S(O)—R³, —S(O₂)—R³, substituted or unsubstituted C₂-C₂₀ heteroalkyl, or NR³R⁴; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

11. The method of claim 10, wherein the carborane or carborane analog comprises a compound defined by Formula XIIA, or a pharmaceutically acceptable salt thereof 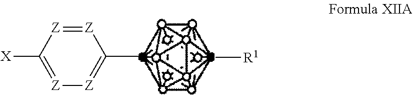
wherein
● is a carbon atom;
∘ is B—H, B-halogen, B-alkyl, B—OH, or B—NH₂;
X is OH, NHR², SH, or S(O)(O)NHR²;
Z is, individually for each occurrence, N or CH, with the proviso that at least one of Z is N;
R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)N R³R⁴, —S(O)—R³, —S(O₂)—R³, substituted or unsubstituted C₂-C₂₀ heteroalkyl, or NR³R⁴;
R² is H, OH, halogen, or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

12. The method of claim 11, wherein the carborane or carborane analog comprises a compound defined by one of the formulae below, or a pharmaceutically acceptable salt thereof: 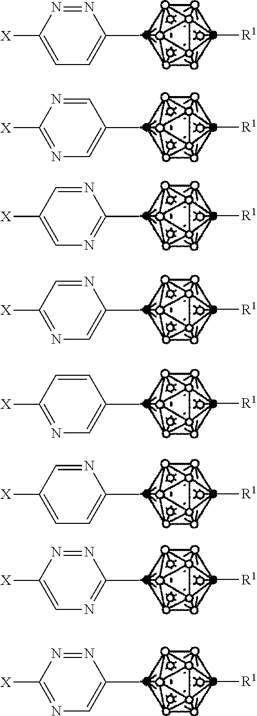
wherein
● is a carbon atom;
∘ is B—H, B-halogen, B-alkyl, B—OH, or B—NH₂;
X is OH, NHR², SH, or S(O)(O)NHR²;
R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)N R³R⁴, —S(O)—R³, —S(O₂)—R³, substituted or unsubstituted C₂-C₂₀ heteroalkyl, or NR³R⁴;
R² is H, OH, halogen, or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

13. The method of claim 11, wherein the carborane or carborane analog comprises a compound defined by one of Formula XIIB-XIIF, or a pharmaceutically acceptable salt thereof: 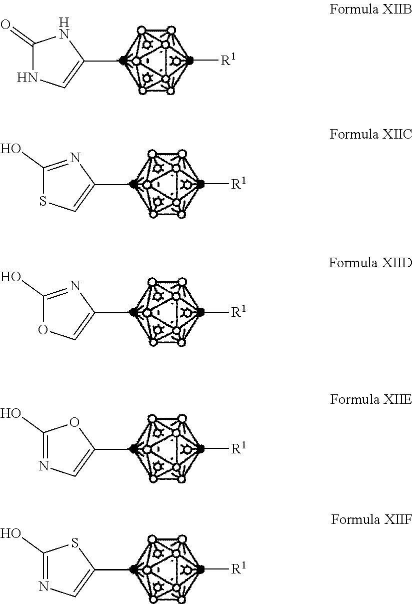
wherein
● is a carbon atom;
∘ is B—H, B-halogen, B-alkyl, B—OH, or B—NH₂;
R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)N R³R⁴, —S(O)—R³, —S(O₂)—R³, substituted or unsubstituted C₂-C₂₀ heteroalkyl, or NR³R⁴;
R² is H, OH, halogen, or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

14. The method of claim 1, wherein the carborane or carborane analog comprises a compound defined by one of the formulae below, or a pharmaceutically acceptable salt thereof: 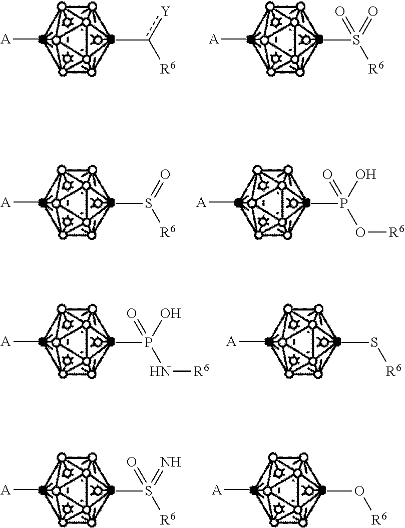
wherein
● is a carbon atom;
∘ is B—H, B-halogen, B-alkyl, B—OH, or B—NH₂;
the dotted line to Y indicates that the bond can be a single bond or a double bond, as valence permits;
A is a substituted or unsubstituted heteroaryl ring;
Y, when present, is O, halogen, OR^{2′}, NHR², SH, or S(O)(O)NHR²;
R⁶ is substituted or unsubstituted C₁-C₁₉ alkyl, substituted or unsubstituted C₂-C₁₉ alkenyl, substituted or unsubstituted C₂-C₁₉ alkynyl, substituted or unsubstituted C₂-C₁₉ alkylaryl, substituted or unsubstituted C₂-C₁₉ alkylheteroaryl, substituted or unsubstituted C₄-C₁₉ alkylcycloalkyl, substituted or unsubstituted C₄-C₁₉ alkylheterocycloalkyl, and substituted or unsubstituted C₂-C₂₀ heteroalkyl. or NR³R⁴;
R² is H, OH, halogen, or substituted or unsubstituted C₁-C₄ alkyl;
R^{2′} is H or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

15. The method of claim 14, wherein A is a five-membered substituted or unsubstituted heteroaryl ring, such as a thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, or 1,3,4-oxadiazolyl ring.

16. The method of claim 14, wherein A is a six-membered substituted or unsubstituted heteroaryl ring, such as a pyridyl, pyrazinyl, pyrimidinyl, triazinyl, or pyridazinyl ring.

17. The method of claim 1, wherein the carborane or carborane analog comprises a compound defined by Formula XIV, or a pharmaceutically acceptable salt thereof
A-Q-R¹ Formula XIV
wherein
A is a substituted or unsubstituted aryl ring or a substituted or unsubstituted heteroaryl ring;
Q is a spacer group chosen from one of the following: 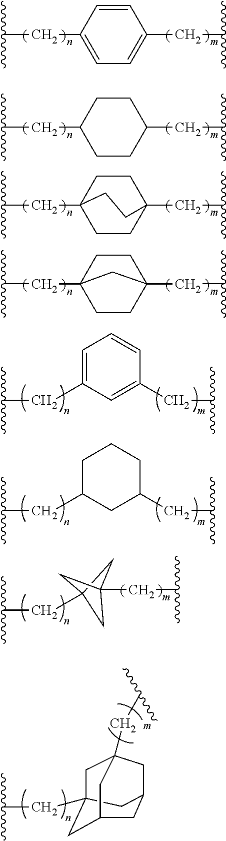
where m and n are each individually 0, 1, 2, or 3;
R¹ is substituted or unsubstituted C₄-C₂₀ alkyl, substituted or unsubstituted C₄-C₂₀ heteroalkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)N R³R⁴, or NR³R⁴; and
R³ and R⁴ are independently selected from substituted or unsubstituted C₁-C₂₀ alkyl, substituted or unsubstituted C₁-C₂₀ heteroalkyl, substituted or unsubstituted C₂-C₂₀ alkenyl, substituted or unsubstituted C₂-C₂₀ alkynyl, substituted or unsubstituted C₂-C₂₀ alkylaryl, or substituted or unsubstituted C₄-C₂₀ alkylcycloalkyl.

18. The method of claim 17, wherein A is a five-membered substituted or unsubstituted heteroaryl ring, such as a thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, or 1,3,4-oxadiazolyl ring.

19. The method of claim 17, wherein A is a six-membered substituted or unsubstituted heteroaryl ring, such as a pyridyl, pyrazinyl, pyrimidinyl, triazinyl, or pyridazinyl ring.

20. The method of claim 17, wherein A is 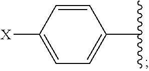
X is OH, NHR², SH, or S(O)(O)NHR²; and R² is H, OH, halogen, or substituted or unsubstituted C₁-C₄ alkyl.

21. The method of claim 17, wherein A is 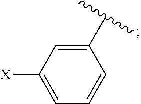
X is OH, NHR², SH, or S(O)(O)NHR²; and R² is H, OH, halogen, or substituted or unsubstituted C₁-C₄ alkyl.

22. The method of claim 17, wherein R¹ is one of the following 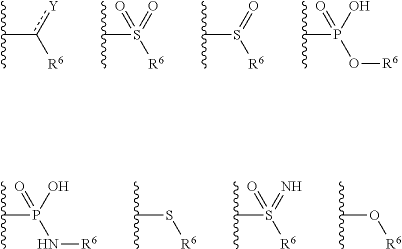
wherein
the dotted line to Y indicates that the bond can be a single bond or a double bond, as valence permits;
Y, when present, is O, halogen, OR^{2′}, NHR², SH, or S(O)(O)NHR²;
R⁶ is substituted or unsubstituted C₁-C₁₉ alkyl, substituted or unsubstituted C₂-C₁₉ alkenyl, substituted or unsubstituted C₂-C₁₉ alkynyl, substituted or unsubstituted C₂-C₁₉ alkylaryl, substituted or unsubstituted C₂-C₁₉ alkylheteroaryl, substituted or unsubstituted C₄-C₁₉ alkylcycloalkyl, substituted or unsubstituted C₄-C₁₉ alkylheterocycloalkyl, and substituted or unsubstituted C₂-C₂₀ heteroalkyl. or NR³R⁴;
R² is H, OH, halogen, or substituted or unsubstituted C₁-C₄ alkyl;
R^{2′} is H or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

23. The method of claim 1, wherein the carborane or carborane analog comprises a selective ERβ agonist.

24. The method of claim 1, wherein the carborane or carborane analog has an EC₅₀ of 800 nM or less at estrogen receptor beta (ERβ).

25. The method of claim 1, wherein the carborane or carborane analog has an ERβ-to-ERα agonist ratio of 8 or more.

26. The method of claim 2, wherein treating the fibrotic condition comprises reducing or inhibiting one or more of: formation or deposition of tissue fibrosis; or reducing the size, cellularity, composition, cellular or collagen content, of a fibrotic lesion.

27. The method of claim 2, wherein the fibrotic condition is a fibrotic condition of the lung, a fibrotic condition of the liver, a fibrotic condition of the heart or vasculature, a fibrotic condition of the kidney, a fibrotic condition of the skin, a fibrotic condition of the gastrointestinal tract, a fibrotic condition of the bone marrow or hematopoietic tissue, a fibrotic condition of the nervous system, or a combination thereof.

28. The method of claim 2, wherein the fibrotic condition is secondary to an infectious disease, an inflammatory disease, an autoimmune disease, a connective disease, a malignant disorder, or a clonal proliferative disorder; a toxin; an environmental hazard, cigarette smoking, a wound; or a medical treatment chosen from a surgical incision, chemotherapy, or radiation.

29-43. (canceled)

44. A compound defined by Formula XI, or a pharmaceutically acceptable salt thereof 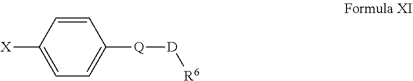
wherein
Q is a substituted or unsubstituted dicarba-closo-dodecaborane cluster;
D is —S—, —S(O)—, —S(O)(O)—, —S(O)(NH)—, —P(O)(OH)O—, —P(O)(OH)NH—, or —O—;
X is OH, NHR², SH, or S(O)(O)NHR²;
R⁶ is substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₂-C₂₀ alkylheteroaryl, substituted or unsubstituted C₄-C₂₀ alkylcycloalkyl, or substituted or unsubstituted C₄-C₂₀ alkylheterocycloalkyl; and
R² is H, OH, halogen, or substituted or unsubstituted C₁-C₄ alkyl.

45-64. (canceled)

65. A compound defined by one of the formulae below, or a pharmaceutically acceptable salt thereof: 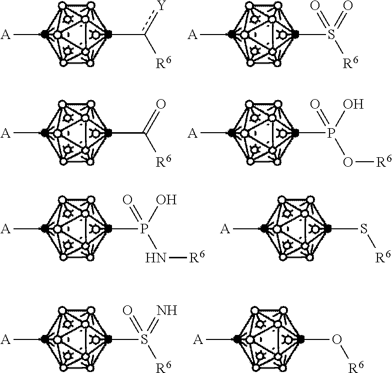
wherein
● is a carbon atom;
∘ is B—H, B-halogen, B-alkyl, B—OH, or B—NH₂;
the dotted line to Y indicates that the bond can be a single bond or a double bond, as valence permits;
A is a substituted or unsubstituted heteroaryl ring;
Y, when present, is O, halogen, OR^{2′}, NHR², SH, or S(O)(O)NHR²;
R⁶ is substituted or unsubstituted C₁-C₁₉ alkyl, substituted or unsubstituted C₂-C₁₉ alkenyl, substituted or unsubstituted C₂-C₁₉ alkynyl, substituted or unsubstituted C₂-C₁₉ alkylaryl, substituted or unsubstituted C₂-C₁₉ alkylheteroaryl, substituted or unsubstituted C₄-C₁₉ alkylcycloalkyl, substituted or unsubstituted C₄-C₁₉ alkylheterocycloalkyl, and substituted or unsubstituted C₂-C₂₀ heteroalkyl. or NR³R⁴;
R² is H, OH, halogen, or substituted or unsubstituted C₁-C₄ alkyl;
R^{2′} is H or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

66-110. (canceled)
